(19) United States
(12) Patent Application Publication
(10) Pub. No.: US 2024/0024508 A1
(43) Pub. Date: Jan. 25, 2024

(54) FORMULATIONS FOR SUPRACHOROIDAL ADMINISTRATION SUCH AS HIGH VISCOSITY FORMULATIONS

(71) Applicant: REGENXBIO Inc., Rockville, MD (US)

(72) Inventors: Jared Bee, Gaithersburg, MD (US); Tristan James Marshall, Potomac, MD (US); Sherri Van Everen, Menlo Park, CA (US); Stephen Joseph Pakola, Irvington, NY (US); Nicholas Alexander Piers Sascha Buss, Rockville, MD (US); Anthony Ray O'Berry, Clarksburg, MD (US); Jesse I. Yoo, Atlanta, GA (US); Ewa Budzynski, Deforest, WI (US)

(21) Appl. No.: 18/030,630

(22) PCT Filed: Oct. 6, 2021
(86) PCT No.: PCT/US2021/053759

§ 371 Date: Apr. 6, 2023

Related U.S. Application Data
(60) Provisional application No. 63/147,527, filed on Feb. 9, 2021.
(60) Provisional application No. 63/088,826, filed on Oct. 7, 2020.

(51) Int. Cl. A61K 48/00; C12N 15/86; C07K 16/22; A61K 47/38; A61K 9/00

(52) U.S. Cl. CPC A61K 48/005; C12N 15/86; A61K 48/0075; C07K 16/22; A61K 47/38; A61K 9/0048; A61K 9/0019; C12N 2750/14143

(57) ABSTRACT

Provided herein are pharmaceutical compositions for administration to a suprachoroidal space of an eye of a subject. The pharmaceutical compositions can include a recombinant adeno-associated virus (AAV) encoding a transgene. Also provided herein are methods for treating or preventing a disease in a subject by administering a therapeutically effective amount of the pharmaceutical compositions to the subject in need.

### PRIORITY

This application claims the benefit of priority to U.S. Ser. No. 63/088,826, filed Oct. 7, 2020, and U.S. Ser. No. 63/147,527, filed Feb. 9, 2021, each of which is incorporated herein by reference in its entirety.

### REFERENCE TO SEQUENCE LISTING SUBMITTED ELECTRONICALLY

This application incorporates by reference a Sequence Listing submitted with this application as text file entitled “12656-141-228_Sequence_Listing.txt” created on Sep. 30, 2021 and having a size of 107,133 bytes.

### 1. BACKGROUND OF THE INVENTION

The human eye is a highly intricate and highly developed sensory organ, which is prone to a host of diseases and disorders. About 285 million people in the world are visually impaired, of whom 39 million are blind and 246 million have moderate to severe visual impairment (World Health Organization, 2012, “Global Data On Visual Impairments 2010,” Geneva: World Health Organization). Some of the leading causes of blindness are cataract (47%), glaucoma (12%), age-related macular degeneration (AMD) (9%), and diabetic retinopathy (5%) (World Health Organization, 2007, “Global Initiative For The Elimination Of Avoidable Blindness: Action Plan 2006-2011,” Geneva: World Health Organization).

Gene therapy has been employed in treating certain eye diseases (see, e.g. International Patent Application No. PCT/US2017/027650 (International Publication No. WO 2017/181021 A1)). Adeno-associated viruses (AAV) are an attractive tool for gene therapy due to properties of non-pathogenicity, broad host and cell type tropism range of infectivity, including both dividing and non-dividing cells, and ability to establish long-term transgene expression (e.g., Gonsalves, 2005, Virology Journal, 2:43).

Current methods used for ocular gene therapy (e.g., by intravitreous or subretinal administrations) are invasive and have serious setbacks, such as, increased risk of cataract, retinal detachment, and separation of photoreceptors from the retinal pigment epithelium (RPE) in the fovea. There is a significant unmet medical need for therapies that improve or eliminate the setbacks from current ocular gene therapy.

Adeno-associated virus (AAV), a member of the Parvoviridae family designated Dependovirus, is a small nonenveloped, icosahedral virus with single-stranded linear DNA genomes of approximately 4.7-kilobases (kb) to 6 kb. The properties of non-pathogenicity, broad host and cell type tropism range of infectivity, including both dividing and non-dividing cells, and ability to establish long-term transgene expression make AAV an attractive tool for gene therapy (e.g., Gonsalves, 2005, Virology Journal, 2:43).

Construct II is being investigated as a treatment delivered by injection into the suprachoroidal space. The suprachoroidal space (SCS) is a region between the sclera and the choroid that expands upon injection of the drug solution (Habot-Wilner, 2019). The SCS space recovers to its pre-injection size as the injected solution is cleared by physiologic processes. The drug solution diffuses within SCS and is absorbed into adjacent tissues. Capillaries in the choroid are permeable to low molecular weight osmolytes. The present disclosure addresses an unmet need of providing pharmaceutical compositions that lead to longer residence time in the suprachoroidal space, and consequently improved efficacy.

### 2. SUMMARY OF THE INVENTION

In one aspect provided herein is a pharmaceutical composition suitable for administration to the suprachoroidal space (SCS) of an eye of a human subject, wherein the pharmaceutical composition comprises a recombinant adeno-associated virus (AAV) vector comprising an expression cassette encoding a transgene, and wherein the pharmaceutical composition has viscosity of between about 25 cP to about 3×10⁶ cP as measured at a shear rate of at most about 1 s⁻¹.

In one aspect provided herein is a pharmaceutical composition suitable for administration to the suprachoroidal space (SCS) of an eye of a human subject, wherein the pharmaceutical composition comprises a recombinant adeno-associated virus (AAV) vector comprising an expression cassette encoding a transgene, wherein the transgene is an anti-human vascular endothelial growth factor (anti-VEGF) antibody, and wherein the pharmaceutical composition has viscosity of between about 25 cP to about 3×10⁶ cP as measured at a shear rate of at most about 1 s⁻¹.

In some embodiments, the clearance time after suprachoroidal administration is equal to or greater than the clearance time of a reference pharmaceutical composition after suprachoroidal administration, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹. In some embodiments, a circumferential spread after suprachoroidal administration is smaller as compared to a circumferential spread of a reference pharmaceutical composition after suprachoroidal administration, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹. In some embodiments, a thickness at a site of injection after suprachoroidal administration is equal to or higher as compared to a thickness at a site of injection after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹. In some embodiments, an expression level of the transgene is detected in the eye for a longer period of time after suprachoroidal administration as compared to a period of time that an expression level of the transgene is detected in the eye after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹. In some embodiments, the concentration of the transgene in the eye after suprachoroidal administration is equal to or higher as compared to the concentration of the transgene in the eye after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹. In some embodiments, the rate of transduction at a site of injection after suprachoroidal administration is equal to or higher as compared to the rate of transduction at a site of injection after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹.

In some embodiments, a level of VEGF-induced vasodilation and/or vascular leakage after suprachoroidal administration is equal to or decreased as compared to a level of VEGF-induced vasodilation and/or vascular leakage after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹.

In some embodiments, the recombinant AAV is Construct II. In some embodiments, the transgene is an anti-human vascular endothelial growth factor (anti-VEGF) antibody. In some embodiments, the recombinant AAV comprises components from one or more adeno-associated virus serotypes selected from the group consisting of AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the recombinant AAV is AAV8. In some embodiments, the recombinant AAV is AAV9.

In some embodiments, the pharmaceutical composition has a viscosity of between about 25 cP to about 100,000 cP, between about 25 cP to about 50,000 cP, between about 25 cP to about 1×10⁴ cP, between about 25 cP to about 5,000 cP, between about 25 cP to about 1×10³ cP, between about 100 cP to about 100,000 cP, between about 100 cP to about 1×10⁴ cP, between about 100 cP to about 5,000 cP, between about 100 cP to about 1×10³ cP, as measured at a shear rate of at most about 1 s⁻¹. In some embodiments, the pharmaceutical composition has viscosity of at least about 100 cP, at least about 400 cP, at least about 500 cP, at least about 900 cP, at least about 1000 cP, at least about 4000 cP, or at least about 1×10⁶ cP, as measured at a shear rate of at most about 1 s⁻¹. In some embodiments, the pharmaceutical composition has viscosity of about 4000 cP as measured at a shear rate of at most about 1 s⁻¹. In some embodiments, the pharmaceutical composition has viscosity of about or greater than about 500 cP as measured at a shear rate of at most about 1 s⁻¹.

In some embodiments, the pharmaceutical composition comprises sucrose. In some embodiments, the pharmaceutical composition does not comprise sucrose. In some embodiments, the pharmaceutical composition comprises at least one of sucrose, 4% sucrose, 6% sucrose, 10% sucrose, 2% carboxymethyl cellulose sodium salt, 1% carboxymethyl cellulose sodium salt, carboxymethyl cellulose (CMC), 0.5% CMC, 1% CMC, 2% CMC, 4% CMC, polyvinyl alcohol, hydroxyethyl cellulose, carboxymethyl cellulose sodium salt, and hydroxypropyl methylcellulose. In some embodiments, the pharmaceutical composition comprises 4% sucrose, 6% sucrose, or 10% sucrose.

In some embodiments, the circumferential spread after suprachoroidal administration of the pharmaceutical composition is smaller by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%. In some embodiments, the clearance time after suprachoroidal administration of the pharmaceutical composition is greater by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or at least 500%. In some embodiments, the clearance time after suprachoroidal administration of the pharmaceutical composition is of about 30 minutes to about 20 hours, about 2 hours to about 20 hours, about 30 minutes to about 24 hours, about 1 hour to about 2 hours, about 30 minutes to about 90 days, about 30 minutes to about 60 days, about 30 minutes to about days, about 30 minutes to about 21 days, about 30 minutes to about 14 days, about 30 minutes to about 7 days, about 30 minutes to about 3 days, about 30 minutes to about 2 days, about 30 minutes to about 1 day, about 4 hours to about 90 days, about 4 hours to about 60 days, about 4 hours to about 30 days, about 4 hours to about 21 days, about 4 hours to about 14 days, about 4 hours to about 7 days, about 4 hours to about 3 days, about 4 hours to about 2 days, about 4 hours to about 1 day, about 4 hours to about 8 hours, about 4 hours to about 16 hours, about 4 hours to about 20 hours, about 1 day to about 90 days, about 1 day to about 60 days, about 1 day to about 30 days, about 1 day to about 21 days, about 1 day to about 14 days, about 1 day to about 7 days, about 1 day to about 3 days, about 2 days to about 90 days, about 3 days to about days, about 3 days to about 60 days, about 3 days to about 30 days, about 3 days to about 21 days, about 3 days to about 14 days, or about 3 days to about 7 days. In some embodiments, the clearance time after suprachoroidal administration of the pharmaceutical composition is not prior to about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days, 3 days, 4 days, 5 days, 6 days, 7 days, 8 days, 9 days, 10 days, 11 days, 12 days, 13 days, 14 days, days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, days, 95 days, 100 days, 120 days, 140 days, 160 days, 180 days, 200 days, 220 days, 240 days, 260 days, 280 days, 300 days, 320 days, 340 days, 360 days, 380 days, or 400 days. In some embodiments, the clearance time of the reference pharmaceutical composition after suprachoroidal administration is of at most about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days, 3 days, 4 days, 5 days, 6 days, 7 days, 8 days, 9 days, 10 days, 11 days, 12 days, 13 days, or 14 days.

In some embodiments, the clearance time is from the SCS or from the eye. In some embodiments, the thickness at the site of injection after suprachoroidal administration of the pharmaceutical composition is higher by at least 2 times, at least 3 times, at least 4 times, at least times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%. In some embodiments, the thickness at the site of injection after suprachoroidal administration of the pharmaceutical composition is about 500 μm to about 3.0 mm, 750 μm to about 2.8 mm, about 750 μm to about 2.5 mm, about 750 μm to about 2 mm, or about 1 mm to about 2 mm. In some embodiments, the thickness at the site of injection after suprachoroidal administration of the pharmaceutical composition is of at least about 50 μm, 100 μm, 200 μm, 300 μm, 400 μm, 500 μm, 600 μm, 700 μm, 800 μm, 900 μm, 1000 μm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, or 10 mm.

In some embodiments, the thickness at the site of injection after suprachoroidal administration of the reference pharmaceutical composition is of at most about 1 nm, 5 nm, 10 nm, 25 nm, 50 nm, 100 nm, 200 nm, 300 nm, 400 nm, 500 nm, 600 nm, 700 nm, 800 nm, 900 nm, 1 μm, 5 μm, 10 μm, 15 μm, 20 μm, 25 μm, 30 μm, 35 μm, 40 μm, 50 μm, 100 μm, 200 μm, 300 μm, 400 μm, 500 μm, 600 μm, 700 μm, 800 μm, 900 μm, or 1000 μm.

In some embodiments, the thickness at the site of injection after suprachoroidal administration of the pharmaceutical composition persists for at least two hours, at least three hours, at least four hours, at least five hours, at least six hours, at least seven hours, at least eight hours, at least ten hours, at least twelve hours, at least eighteen hours, at least twenty-four hours, at least two days, at least three days, at least five days, at least ten days, at least twenty-one days, at least one month, at least six weeks, at least two months, at least three months, at least 4 months, at least 5 months, at least 6 months, at least 9 months, at least one year, at least three years, or at least five years.

In some embodiments, the concentration of the transgene in the eye after suprachoroidal administration of the pharmaceutical composition is higher by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%. In some embodiments, the longer period of time after suprachoroidal administration of the pharmaceutical composition is longer by at least 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

In some embodiments, the transgene is detected in the eye after suprachoroidal administration of the pharmaceutical composition for at least about 1 day, 2 days 3 days, 4 days, days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days. In some embodiments, the transgene is detected in the eye after suprachoroidal administration of the reference pharmaceutical composition for at most about 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, days, 60 days, 65 days, 70 days, 75 days, 80 days, 85 days, 90 days, 95 days, or 100 days, 120 days, 140 days, 160 days, 180 days, 200 days, 220 days, 240 days, 260 days, 280 days, 300 days, 320 days, 340 days, 360 days, 380 days, or 400 days after.

In some embodiments, a level of VEGF-induced vasodilation and/or vascular leakage after suprachoroidal administration of the pharmaceutical composition is equal to or decreased as compared to a level of VEGF-induced vasodilation and/or vascular leakage after suprachoroidal administration of the reference pharmaceutical composition. In some embodiments, the level of VEGF-induced vasodilation and/or vascular leakage after suprachoroidal administration of the pharmaceutical composition is decreased by at least about 2 times, at least 3 times, at least 4 times, at least 5 times, at least 6 times, at least 7 times, at least 8 times, at least 9 times, at least times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%. In some embodiments, the rate of transduction at the site of injection after suprachoroidal administration of the pharmaceutical composition is higher by at least about 2 times, at least 3 times, at least 4 times, at least 5 times, at least 6 times, at least 7 times, at least 8 times, at least 9 times, at least times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

In some embodiments, the recombinant AAV stability in the pharmaceutical composition is at least about 50% the recombinant AAV stability in the reference pharmaceutical composition. In some embodiments, the recombinant AAV stability is determined by infectivity of the recombinant AAV. In some embodiments, the recombinant AAV stability is determined by a level of aggregation of the recombinant AAV. In some embodiments, the recombinant AAV stability is determined by a level of free DNA released by the recombinant AAV. In some embodiments, the pharmaceutical composition comprises about 50% more, about 25% more, about 15% more, about 10% more, about 5% more, about 4% more, about 3% more, about 2% more, about 1% more, about 0% more, about 1% less, about 2% less, about 5% less, about 7% less, about 10% less, about 2 times more, about 3 times more, about 2 times less, about 3 times less, free DNA as compared to a level of free DNA in the reference pharmaceutical composition. In some embodiments, the recombinant AAV in the pharmaceutical composition has an infectivity that is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times higher as compared to the infectivity of the recombinant AAV in the reference pharmaceutical composition. In some embodiments, the pharmaceutical composition comprises at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times less recombinant AAV aggregation as compared to a level of the recombinant AAV aggregation in the reference pharmaceutical composition.

In some embodiments, the transgene is a transgene suitable to treat, or otherwise ameliorate, prevent or slow the progression of a disease of interest. In some embodiments, the human subject is diagnosed with nAMD (wet AMD), dry AMD, retinal vein occlusion (RVO), diabetic macular edema (DME), or diabetic retinopathy (DR), or Batten disease. In some embodiments, the human subject is diagnosed with mucopolysaccharidosis type IVA (MPS IVA), mucopolysaccharidosis type I (MPS I), mucopolysaccharidosis type II (MPS II), familial hypercholesterolemia (FH), homozygous familial hypercholesterolemia (HoFH), coronary artery disease, cerebrovascular disease, Duchenne muscular dystrophy, Limb Girdle muscular dystrophy, Becker muscular dystrophy and sporadic inclusion body myositis, or kallikrein-related disease. In some embodiments, the AAV encodes Palmitoyl-Protein Thioesterase 1 (PPT1) or Tripeptidyl-Peptidase 1 (TPP1).

In some embodiments, the amount of the recombinant AAV genome copies is based on a vector genome concentration. In some embodiments, the amount of the recombinant AAV genome copies is based on genome copies per administration. In some embodiments, the amount of the recombinant AAV genome copies is based on total genome copies administered to the human subject. In some embodiments, the genome copies per administration is the genome copies of the recombinant AAV per suprachoroidal administration. In some embodiments, the total genome copies administered is the total genome copies of the recombinant AAV administered suprachoroidally. In some embodiments, the vector genome concentration (VGC) is of about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 2×10¹¹ GC/mL, about 2.4×10¹¹ GC/mL, about 2.5×10¹¹ GC/mL, about 3×10¹¹ GC/mL, about 6.2×10¹¹ GC/mL, about 1×10¹² GC/mL, about 2.5×10¹² GC/mL, about 3×10¹² GC/mL, about 5×10¹² GC/mL, about 1.5×10¹³ GC/mL, about 2×10¹³ GC/mL, or about 3×10¹³ GC/mL. In some embodiments, the total genome copies administered is about 6.0×10¹⁰ genome copies, about 1.6×10¹¹ genome copies, about 2.5×10¹¹ genome copies, about 5.0×10¹¹ genome copies, about 1.5×10¹² genome copies, about 3×10¹² genome copies, about 1.0×10¹² genome copies, about 2.5×10¹² genome copies, or about 3.0×10¹³ genome copies. In some embodiments, the genome copies per administration is about 6.0×10¹⁰ genome copies, about 1.6×10¹¹ genome copies, about 2.5×10¹¹ genome copies, about 5.0×10¹¹ genome copies, about 1.5×10¹² genome copies, about 3×10¹² genome copies, about 1.0×10¹² genome copies, about 2.5×10¹² genome copies, or about 3.0×10¹³ genome copies.

In some embodiments, the pharmaceutical composition is administered once, twice, three times, four times, five times, six times, seven times, eight times, nine times, ten times, fifteen times, twenty times, twenty five times, or thirty times. In some embodiments, the reference pharmaceutical composition is administered once, twice, three times, four times, five times, six times, seven times, eight times, nine times, ten times, fifteen times, twenty times, twenty five times, or thirty times. In some embodiments, the pharmaceutical composition is administered once in one day, twice in one day, three times in one day, four times in one day, five times in one day, six times in one day, or seven times in one day. In some embodiments, the reference pharmaceutical composition is administered once in one day, twice in one day, three times in one day, four times in one day, five times in one day, six times in one day, or seven times in one day.

In some embodiments, the reference pharmaceutical composition comprises DPBS and sucrose. In some embodiments, the reference pharmaceutical composition has a viscosity of about 1 cP as measured at a shear rate of at most about 1 s⁻¹. In some embodiments, the pharmaceutical composition comprises 1% carboxymethyl cellulose high viscosity grade. In some embodiments, the pharmaceutical composition comprises 0.2 to 15% carboxymethyl cellulose (CMC) high viscosity grade, CMC high viscosity grade, CMC medium viscosity grade, hydroxypropyl methylcellulose (HPMC), HPMC, hydroxyethyl cellulose (HES), CMC low viscosity grade, and/or poloxamer 407. In some embodiments, the viscosity of the pharmaceutical composition is measured at a shear rate of 0 s⁻¹. In some embodiments, the viscosity of the reference pharmaceutical composition is measured at a shear rate of 0 s⁻¹. In some embodiments, the viscosity of the pharmaceutical composition and the viscosity of the reference pharmaceutical composition is measured at the same shear rate. In some embodiments, the viscosity of the pharmaceutical composition is shear-thinning.

In some embodiments, the viscosity of the pharmaceutical composition is about, at most about, or at least about 0.1 cP, 0.2 cP, 0.3 cP, 0.4 cP, 0.5 cP, 0.6 cP, 0.7 cP, 0.8 cP, 0.9 cP, 1 cP, 2 cP, 3 cP, 4 cP, 5 cP, 10 cP, 20 cP, 25 cP, 30 cP, 35 cP, 40 cP, 50 cP, 60 cP, 70 cP, 80 cP, cP, 100 cP, 150 cP, 200 cP, 250 cP, 300 cP, 350 cP, 400 cP, 450 cP, 500 cP, 550 cP, 600 cP, 650 cP, 700 cP, 750 cP, 800 cP, 850 cP, 900 cP, 950 cP, 1000 cP, 1500 cP, 2000 cP, 2500 cP, 3000 cP, 3500 cP, 4000 cP, 4500 cP, 5000 cP, or 10000 cP, as measured at a shear rate of at least about 1,000 s⁻¹. In some embodiments, the viscosity of the pharmaceutical composition is measured at a shear rate of at least about 1,000 s⁻¹, 2,000 s⁻¹, 3,000 s⁻¹, 4,000 s⁻¹, 5,000 s⁻¹, 6,000 s⁻¹, 7,000 s⁻¹, 8,000 s⁻¹, 9,000 s⁻¹, 10,000 s⁻¹, 15,000 s⁻¹, 20,000 s⁻¹, or 30,000 s⁻¹. In some embodiments, the viscosity of the pharmaceutical composition is about or at most about 35 cP as measured at a shear rate of about 5,000 s⁻¹. In some embodiments, the viscosity of the pharmaceutical composition is about or at most about 25 cP as measured at a shear rate of about 10,000 s⁻¹. In some embodiments, the viscosity of the pharmaceutical composition is about or at least about 500 cP as measured at a shear rate of at most about 1 s⁻¹. In some embodiments, the viscosity of the pharmaceutical composition is about or at least about 1500 cP as measured at a shear rate of at most about 1 s⁻¹. In some embodiments, the viscosity of the pharmaceutical composition is about or at most about 362 cP as measured at a shear rate of at least about 1000 s⁻¹. In some embodiments, the viscosity of the reference pharmaceutical composition is about or at most about 0.1 cP, 0.2 cP, 0.3 cP, 0.4 cP, 0.5 cP, 0.6 cP, 0.7 cP, 0.8 cP, 0.9 cP, 1 cP, 1.1 cP, 1.2 cP, 1.3 cP, 1.4 cP, 1.5 cP, 1.6 cP, 1.7 cP, 1.8 cP, 1.9 cP, 2 cP, 2.1 cP, 2.2 cP, 2.3 cP, 2.4 cP, 2.5 cP, 2.6 cP, 2.7 cP, 2.8 cP, 2.9 cP, 3 cP, 3.1 cP, 3.2 cP, 3.3 cP, 3.4 cP, 3.5 cP, 3.6 cP, 3.7 cP, 3.8 cP, 3.9 cP, 4 cP, 4.1 cP, 4.2 cP, 4.3 cP, 4.4 cP, 4.5 cP, 4.6 cP, 4.7 cP, 4.8 cP, 4.9 cP, or 5 cP as measured at a shear rate of at least about 1000 s⁻¹. In some embodiments, the viscosity of the reference pharmaceutical composition is about or at most about 0.1 cP, 0.2 cP, 0.3 cP, 0.4 cP, 0.5 cP, 0.6 cP, 0.7 cP, 0.8 cP, 0.9 cP, 1 cP, 1.1 cP, 1.2 cP, 1.3 cP, 1.4 cP, 1.5 cP, 1.6 cP, 1.7 cP, 1.8 cP, 1.9 cP, 2 cP, 2.1 cP, 2.2 cP, 2.3 cP, 2.4 cP, 2.5 cP, 2.6 cP, 2.7 cP, 2.8 cP, 2.9 cP, 3 cP, 3.1 cP, 3.2 cP, 3.3 cP, 3.4 cP, 3.5 cP, 3.6 cP, 3.7 cP, 3.8 cP, 3.9 cP, 4 cP, 4.1 cP, 4.2 cP, 4.3 cP, 4.4 cP, 4.5 cP, 4.6 cP, 4.7 cP, 4.8 cP, 4.9 cP, or 5 cP as measured at a shear rate of at most about 1 s⁻¹. In some embodiments, the viscosity of the pharmaceutical composition is about 0.5 cP to about 400 cP as measured at a shear rate of at least about 1000 s⁻¹.

In some embodiments, the pharmaceutical composition comprises modified Dulbecco's phosphate-buffered saline solution, and optionally a surfactant. In some embodiments, a surfactant is poloxamer 188, polysorbate 20, and/or polysorbate 80. In some embodiments, the pharmaceutical composition comprises potassium chloride, potassium phosphate monobasic, sodium chloride, sodium phosphate dibasic anyhydrous, sucrose, and optionally a surfactant. In some embodiments, the pharmaceutical composition comprises 0.2 mg/mL potassium chloride, 0.2 mg/mL potassium phosphate monobasic, 5.84 mg/mL sodium chloride, 1.15 mg/mL sodium phosphate dibasic anyhydrous, 40.0 mg/mL (4% w/v) sucrose, and optionally a surfactant. In some embodiments, the pharmaceutical composition comprises potassium chloride, potassium phosphate monobasic, sodium chloride, sodium phosphate dibasic anyhydrous, sucrose, optionally one or more surfactants selected from poloxamer 188, polysorbate 20, and polysorbate 80, and optionally one or more viscosity modifiers selected from CMC high viscosity grade, CMC medium viscosity grade, CMC low viscosity grade, hydroxypropyl methylcellulose (HPMC), hydroxyethyl cellulose (HES), and poloxamer 407. In some embodiments, the pharmaceutical composition comprises potassium chloride, potassium phosphate monobasic, sodium chloride, sodium phosphate dibasic anyhydrous, sucrose, optionally one or more surfactants selected from poloxamer 188, polysorbate 20, and polysorbate and optionally one or more viscosity modifiers selected from 0.5% CMC high viscosity grade, 1% CMC high viscosity grade, 0.5% CMC medium viscosity grade, CMC low viscosity grade, 0.5% hydroxypropyl methylcellulose (HPMC), 0.2% HPMC, 2% hydroxyethyl cellulose (HES), and 15% poloxamer 407. In some embodiments, the pharmaceutical composition comprises potassium chloride, potassium phosphate monobasic, sodium chloride, sodium phosphate dibasic anyhydrous, sucrose, one or more surfactants selected from poloxamer 188, polysorbate 20, and polysorbate 80, and one or more polysaccharides selected from CMC, HPMC, and HES. In some embodiments, the pharmaceutical composition comprises 0.2 mg/mL potassium chloride, 0.2 mg/mL potassium phosphate monobasic, 5.84 mg/mL sodium chloride, 1.15 mg/mL sodium phosphate dibasic anyhydrous, 40.0 mg/mL (4% w/v) sucrose, 0.001% (0.01 mg/mL) poloxamer 188 and 1% carboxymethyl cellulose (CMC) high viscosity grade. In some embodiments, the pharmaceutical composition is stored at about room temperature, 20° C., 4° C., or −80° C. In some embodiments, the pharmaceutical composition is stored prior to administration to a human subject.

### 2.1 ILLUSTRATIVE EMBODIMENTS

1. A pharmaceutical composition suitable for administration to the suprachoroidal space (SCS) of an eye of a human subject, wherein the pharmaceutical composition comprises a recombinant adeno-associated virus (AAV) vector comprising an expression cassette encoding a transgene, and wherein the pharmaceutical composition has viscosity of between about 25 cP to about 3×10⁶ cP as measured at a shear rate of at most about 1 s⁻¹.
2. A pharmaceutical composition suitable for administration to the suprachoroidal space (SCS) of an eye of a human subject, wherein the pharmaceutical composition comprises a recombinant adeno-associated virus (AAV) vector comprising an expression cassette encoding a transgene, wherein the transgene is an anti-human vascular endothelial growth factor (anti-VEGF) antibody, and wherein the pharmaceutical composition has viscosity of between about 25 cP to about 3×10⁶ cP as measured at a shear rate of at most about 1 s⁻¹.
3. The pharmaceutical composition of paragraphs 1 or 2, wherein the clearance time after suprachoroidal administration is equal to or greater than the clearance time of a reference pharmaceutical composition after suprachoroidal administration, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹.
4. The pharmaceutical composition of paragraphs 1 or 2, wherein a circumferential spread after suprachoroidal administration is smaller as compared to a circumferential spread of a reference pharmaceutical composition after suprachoroidal administration, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹.
5. The pharmaceutical composition of paragraphs 1 or 2, wherein a thickness at a site of injection after suprachoroidal administration is equal to or higher as compared to a thickness at a site of injection after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹.
6. The pharmaceutical composition of paragraphs 1 or 2, wherein an expression level of the transgene is detected in the eye for a longer period of time after suprachoroidal administration as compared to a period of time that an expression level of the transgene is detected in the eye after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹.
7. The pharmaceutical composition of paragraphs 1 or 2, wherein the concentration of the transgene in the eye after suprachoroidal administration is equal to or higher as compared to the concentration of the transgene in the eye after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹.
8. The pharmaceutical composition of paragraphs 1 or 2, wherein the rate of transduction at a site of injection after suprachoroidal administration is equal to or higher as compared to the rate of transduction at a site of injection after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹.
9. The pharmaceutical composition of paragraph 2, wherein a level of VEGF-induced vasodilation and/or vascular leakage after suprachoroidal administration is equal to or decreased as compared to a level of VEGF-induced vasodilation and/or vascular leakage after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹.
10. The pharmaceutical composition of any one of paragraphs 1-9, wherein the recombinant AAV is Construct II.
11. The pharmaceutical composition of any one of paragraphs 1, 3-8 and 10, wherein the transgene is an anti-human vascular endothelial growth factor (anti-VEGF) antibody.
12. The pharmaceutical composition of any one of paragraphs 1-11, wherein the recombinant AAV comprises components from one or more adeno-associated virus serotypes selected from the group consisting of AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.
13. The pharmaceutical composition of any one of paragraphs 1-12, wherein the recombinant AAV is AAV8.
14. The pharmaceutical composition of any one of paragraphs 1-9 and 11-12, wherein the recombinant AAV is AAV9.
15. The pharmaceutical composition of any one of paragraphs 1-14, wherein the pharmaceutical composition has a viscosity of between about 25 cP to about 100,000 cP, between about 25 cP to about 50,000 cP, between about 25 cP to about 1×10⁴ cP, between about cP to about 5,000 cP, between about 25 cP to about 1×10³ cP, between about 100 cP to about 100,000 cP, between about 100 cP to about 1×10⁴ cP, between about 100 cP to about 5,000 cP, between about 100 cP to about 1×10³ cP, as measured at a shear rate of at most about 1 s⁻¹.
16. The pharmaceutical composition of any one of paragraphs 1-15, wherein the pharmaceutical composition has viscosity of at least about 100 cP, at least about 400 cP, at least about 500 cP, at least about 900 cP, at least about 1000 cP, at least about 4000 cP, or at least about 1×10⁶ cP, as measured at a shear rate of at most about 1 s⁻¹.
17. The pharmaceutical composition of any one of paragraphs 1-16, wherein the pharmaceutical composition has viscosity of about or greater than about 500 cP as measured at a shear rate of at most about 1 s⁻¹.
18. The pharmaceutical composition of any one of paragraphs 1-17, wherein the pharmaceutical composition comprises sucrose.
19. The pharmaceutical composition of any one of paragraphs 1-17, wherein the pharmaceutical composition does not comprise sucrose.
20. The pharmaceutical composition of any one of paragraphs 1-19, wherein the pharmaceutical composition comprises at least one of sucrose, 4% sucrose, 6% sucrose, 10% sucrose, 2% carboxymethyl cellulose sodium salt, 1% carboxymethyl cellulose sodium salt, carboxymethyl cellulose (CMC), 0.5% CMC, 1% CMC, 2% CMC, 4% CMC, polyvinyl alcohol, hydroxyethyl cellulose, carboxymethyl cellulose sodium salt, and hydroxypropyl methylcellulose.
21. The pharmaceutical composition of any one of paragraphs 1-18 and 20, wherein the pharmaceutical composition comprises 4% sucrose, 6% sucrose, or 10% sucrose.
22. The pharmaceutical composition of any one of paragraphs 4 and 10-21, wherein the circumferential spread after suprachoroidal administration of the pharmaceutical composition is smaller by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.
23. The pharmaceutical composition of any one of paragraphs 3 and 10-22, wherein the clearance time after suprachoroidal administration of the pharmaceutical composition is greater by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or at least 500%.
24. The pharmaceutical composition of any one of paragraphs 1-23, wherein the clearance time after suprachoroidal administration of the pharmaceutical composition is of about minutes to about 20 hours, about 2 hours to about 20 hours, about 30 minutes to about 24 hours, about 1 hour to about 2 hours, about 30 minutes to about 90 days, about 30 minutes to about 60 days, about 30 minutes to about 30 days, about 30 minutes to about 21 days, about 30 minutes to about 14 days, about 30 minutes to about 7 days, about 30 minutes to about 3 days, about 30 minutes to about 2 days, about 30 minutes to about 1 day, about 4 hours to about 90 days, about 4 hours to about 60 days, about 4 hours to about 30 days, about 4 hours to about 21 days, about 4 hours to about 14 days, about 4 hours to about 7 days, about 4 hours to about 3 days, about 4 hours to about 2 days, about 4 hours to about 1 day, about 4 hours to about 8 hours, about 4 hours to about 16 hours, about 4 hours to about 20 hours, about 1 day to about 90 days, about 1 day to about 60 days, about 1 day to about 30 days, about 1 day to about 21 days, about 1 day to about 14 days, about 1 day to about 7 days, about 1 day to about 3 days, about 2 days to about 90 days, about 3 days to about 90 days, about 3 days to about 60 days, about 3 days to about 30 days, about 3 days to about 21 days, about 3 days to about 14 days, or about 3 days to about 7 days.
25. The pharmaceutical composition of any one of paragraphs 1-24, wherein the clearance time after suprachoroidal administration of the pharmaceutical composition is not prior to about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days, 3 days, 4 days, 5 days, 6 days, 7 days, 8 days, 9 days, 10 days, 11 days, 12 days, 13 days, 14 days, days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, days, 95 days, 100 days, 120 days, 140 days, 160 days, 180 days, 200 days, 220 days, 240 days, 260 days, 280 days, 300 days, 320 days, 340 days, 360 days, 380 days, or 400 days.
26. The pharmaceutical composition of any one of paragraphs 3-25, wherein the clearance time of the reference pharmaceutical composition after suprachoroidal administration is of at most about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days, 3 days, 4 days, 5 days, 6 days, 7 days, 8 days, 9 days, 10 days, 11 days, 12 days, 13 days, or 14 days.
27. The pharmaceutical composition of any one of paragraphs 1-26, wherein the clearance time is from the SCS or from the eye.
28. The pharmaceutical composition of any one of paragraphs 5 and 10-27, wherein the thickness at the site of injection after suprachoroidal administration of the pharmaceutical composition is higher by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.
29. The pharmaceutical composition of any one of paragraphs 5 and 10-28, wherein the thickness at the site of injection after suprachoroidal administration of the pharmaceutical composition is about 500 μm to about 3.0 mm, 750 μm to about 2.8 mm, about 750 μm to about 2.5 mm, about 750 μm to about 2 mm, or about 1 mm to about 2 mm.
30. The pharmaceutical composition of any one of paragraphs 5 and 10-29, wherein the thickness at the site of injection after suprachoroidal administration of the pharmaceutical composition is of at least about 50 μm, 100 μm, 200 μm, 300 μm, 400 μm, 500 μm, 600 μm, 700 μm, 800 μm, 900 μm, 1000 μm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, or 10 mm.
31. The pharmaceutical composition of any one of paragraphs 5 and 10-30, wherein the thickness at the site of injection after suprachoroidal administration of the reference pharmaceutical composition is of at most about 1 nm, 5 nm, 10 nm, 25 nm, 50 nm, 100 nm, 200 nm, 300 nm, 400 nm, 500 nm, 600 nm, 700 nm, 800 nm, 900 nm, 1 μm, 5 μm, 10 μm, 15 μm, 20 μm, 25 μm, 30 μm, 35 μm, 40 μm, 50 μm, 100 μm, 200 μm, 300 μm, 400 μm, 500 μm, 600 μm, 700 μm, 800 μm, 900 μm, or 1000 μm.
32. The pharmaceutical composition of any one of paragraphs 5 and 10-31, wherein the thickness at the site of injection after suprachoroidal administration of the pharmaceutical composition persists for at least two hours, at least three hours, at least four hours, at least five hours, at least six hours, at least seven hours, at least eight hours, at least ten hours, at least twelve hours, at least eighteen hours, at least twenty-four hours, at least two days, at least three days, at least five days, at least ten days, at least twenty-one days, at least one month, at least six weeks, at least two months, at least three months, at least 4 months, at least 5 months, at least 6 months, at least 9 months, at least one year, at least three years, or at least five years.
33. The pharmaceutical composition of any one of paragraphs 7 and 10-32, wherein the concentration of the transgene in the eye after suprachoroidal administration of the pharmaceutical composition is higher by at least 2 times, at least 3 times, at least 4 times, at least times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.
34. The pharmaceutical composition of any one of paragraphs 6 and 10-33, wherein the longer period of time after suprachoroidal administration of the pharmaceutical composition is longer by at least 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.
35. The pharmaceutical composition of any one of paragraphs 1-34, wherein the transgene is detected in the eye after suprachoroidal administration of the pharmaceutical composition for at least about 1 day, 2 days 3 days, 4 days, 5 days, 6 days, 7 days, 8 days, 9 days, days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.
36. The pharmaceutical composition of any one of paragraphs 3-35, wherein the transgene is detected in the eye after suprachoroidal administration of the reference pharmaceutical composition for at most about 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, days, 60 days, 65 days, 70 days, 75 days, 80 days, 85 days, 90 days, 95 days, or 100 days, 120 days, 140 days, 160 days, 180 days, 200 days, 220 days, 240 days, 260 days, 280 days, 300 days, 320 days, 340 days, 360 days, 380 days, or 400 days after.
37. The pharmaceutical composition of paragraph 11, wherein a level of VEGF-induced vasodilation and/or vascular leakage after suprachoroidal administration of the pharmaceutical composition is equal to or decreased as compared to a level of VEGF-induced vasodilation and/or vascular leakage after suprachoroidal administration of the reference pharmaceutical composition.
38. The pharmaceutical composition of any one of paragraphs 9-37, wherein the level of VEGF-induced vasodilation and/or vascular leakage after suprachoroidal administration of the pharmaceutical composition is decreased by at least about 2 times, at least 3 times, at least 4 times, at least 5 times, at least 6 times, at least 7 times, at least 8 times, at least 9 times, at least times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.
39. The pharmaceutical composition of any one of paragraphs 8 and 10-38, wherein the rate of transduction at the site of injection after suprachoroidal administration of the pharmaceutical composition is higher by at least about 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.
40. The pharmaceutical composition of any one of paragraphs 1-39, wherein the recombinant AAV stability in the pharmaceutical composition is at least about 50% the recombinant AAV stability in the reference pharmaceutical composition.
41. The pharmaceutical composition of paragraph 40, wherein the recombinant AAV stability is determined by infectivity of the recombinant AAV.
42. The pharmaceutical composition of paragraph 40, wherein the recombinant AAV stability is determined by a level of aggregation of the recombinant AAV.
43. The pharmaceutical composition of paragraph 40, wherein the recombinant AAV stability is determined by a level of free DNA released by the recombinant AAV.
44. The pharmaceutical composition of paragraph 43, wherein the pharmaceutical composition comprises about 50% more, about 25% more, about 15% more, about 10% more, about 5% more, about 4% more, about 3% more, about 2% more, about 1% more, about 0% more, about 1% less, about 2% less, about 5% less, about 7% less, about 10% less, about 2 times more, about 3 times more, about 2 times less, about 3 times less, free DNA as compared to a level of free DNA in the reference pharmaceutical composition.
45. The pharmaceutical composition of paragraph 41, wherein the recombinant AAV in the pharmaceutical composition has an infectivity that is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times higher as compared to the infectivity of the recombinant AAV in the reference pharmaceutical composition.
46. The pharmaceutical composition of paragraph 42, wherein the pharmaceutical composition comprises at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times less recombinant AAV aggregation as compared to a level of the recombinant AAV aggregation in the reference pharmaceutical composition.
47. The pharmaceutical composition of any one of paragraphs 1-46, wherein the transgene is a transgene suitable to treat, or otherwise ameliorate, prevent or slow the progression of a disease of interest.
48. The pharmaceutical composition of any one of paragraphs 1-47, wherein the human subject is diagnosed with nAMD (wet AMD), dry AMD, retinal vein occlusion (RVO), diabetic macular edema (DME), diabetic retinopathy (DR), Batten disease, glaucoma or non-infectious uveitis.
49. The pharmaceutical composition of any one of paragraphs 1-47, wherein the human subject is diagnosed with mucopolysaccharidosis type IVA (MPS IVA), mucopolysaccharidosis type I (MPS I), mucopolysaccharidosis type II (MPS II), familial hypercholesterolemia (FH), homozygous familial hypercholesterolemia (HoFH), coronary artery disease, cerebrovascular disease, Duchenne muscular dystrophy, Limb Girdle muscular dystrophy, Becker muscular dystrophy and sporadic inclusion body myositis, or kallikrein-related disease.
50. The pharmaceutical composition of any one of paragraphs 1, 3-8 and 10-49, wherein the AAV encodes Palmitoyl-Protein Thioesterase 1 (PPT1), Tripeptidyl-Peptidase 1 (TPP1), anti-VEGF antibody or antigen-binding fragment thereof, anti-kallikrein antibody or antigen-binding fragment, anti-TNF antibody or antigen-binding fragment, anti-C3 antibody or antigen-binding fragment, or anti-CS antibody or antigen-binding fragment.
51. The pharmaceutical composition of any one of paragraphs 3-50, wherein the amount of the recombinant AAV genome copies is based on a vector genome concentration.
52. The pharmaceutical composition of any one of paragraphs 3-50, wherein the amount of the recombinant AAV genome copies is based on genome copies per administration
53. The pharmaceutical composition of any one of paragraphs 3-50, wherein the amount of the recombinant AAV genome copies is based on total genome copies administered to the human subject.
54. The pharmaceutical composition of paragraph 52, wherein the genome copies per administration is the genome copies of the recombinant AAV per suprachoroidal administration.
55. The pharmaceutical composition of paragraph 53, wherein the total genome copies administered is the total genome copies of the recombinant AAV administered suprachoroidally.
56. The pharmaceutical composition of paragraph 51, wherein the vector genome concentration (VGC) is of about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 2×10¹¹ GC/mL, about 2.4×10¹¹ GC/mL, about 2.5×10¹¹ GC/mL, about 3×10¹¹ GC/mL, about 6.2×10¹¹ GC/mL, about 1×10¹² GC/mL, about 2.5×10¹² GC/mL, about 3×10¹² GC/mL, about 5×10¹² GC/mL, about 6×10¹² GC/mL, about 1.5×10¹³ GC/mL, about 2×10¹³ GC/mL, or about 3×10¹³ GC/mL.
57. The pharmaceutical composition of any one of paragraphs 53 and 55, wherein the total genome copies administered is about 6.0×10¹⁰ genome copies, about 1.6×10¹¹ genome copies, about 2.5×10¹¹ genome copies, about 3×10¹¹ genome copies, about 5.0×10¹¹ genome copies, about 6×10¹¹ genome copies, about 3×10¹² genome copies, about 1.0×10¹² genome copies, about 1.5×10¹² genome copies, about 2.5×10¹² genome copies, or about 3.0×10¹³ genome copies.
58. The pharmaceutical composition of any one of paragraphs 52 and 54, wherein the genome copies per administration is about 6.0×10¹⁰ genome copies, about 1.6×10¹¹ genome copies, about 2.5×10¹¹ genome copies, about 3×10¹¹ genome copies, about 5.0×10¹¹ genome copies, about 3×10¹² genome copies, about 1.0×10¹² genome copies, about 1.5×10¹² genome copies, about 2.5×10¹² genome copies, or about 3.0×10¹³ genome copies.
59. The pharmaceutical composition of any one of paragraphs 1-58, wherein the pharmaceutical composition is administered once, twice, three times, four times, five times, six times, seven times, eight times, nine times, ten times, fifteen times, twenty times, twenty five times, or thirty times.
60. The pharmaceutical composition of any one of paragraphs 3-59, wherein the reference pharmaceutical composition is administered once, twice, three times, four times, five times, six times, seven times, eight times, nine times, ten times, fifteen times, twenty times, twenty five times, or thirty times.
61. The pharmaceutical composition of any one of paragraphs 1-60, wherein the pharmaceutical composition is administered once in one day, twice in one day, three times in one day, four times in one day, five times in one day, six times in one day, or seven times in one day.
62. The pharmaceutical composition of any one of paragraphs 3-60, wherein the reference pharmaceutical composition is administered once in one day, twice in one day, three times in one day, four times in one day, five times in one day, six times in one day, or seven times in one day.
63. The pharmaceutical composition of any one of paragraphs 1-60, wherein the reference pharmaceutical composition comprises DPBS and sucrose.
64. The pharmaceutical composition of any one of paragraphs 3-60, wherein the reference pharmaceutical composition has a viscosity of about 1 cP as measured at a shear rate of at most about 1 s⁻¹.
65. The pharmaceutical composition of any one of paragraphs 1-64, wherein the pharmaceutical composition comprises 0.2 to 15% carboxymethyl cellulose (CMC) high viscosity grade, CMC high viscosity grade, CMC medium viscosity grade, hydroxypropyl methylcellulose (HPMC), HPMC, hydroxyethyl cellulose (HES), CMC low viscosity grade, and/or poloxamer 407.
66. The pharmaceutical composition of any one of paragraphs 1-65, wherein the viscosity of the pharmaceutical composition is measured at a shear rate of 0 s⁻¹.
67. The pharmaceutical composition of any one of paragraphs 3-66, wherein the viscosity of the reference pharmaceutical composition is measured at a shear rate of 0 s⁻¹.
68. The pharmaceutical composition of any one of paragraphs 1-67, wherein the viscosity of the pharmaceutical composition and the viscosity of the reference pharmaceutical composition is measured at the same shear rate.
69. The pharmaceutical composition of any one of paragraphs 1-68, wherein the viscosity of the pharmaceutical composition is shear-thinning.
70. The pharmaceutical composition of any one of paragraphs 1-69, wherein the viscosity of the pharmaceutical composition is about, at most about, or at least about 0.1 cP, 0.2 cP, 0.3 cP, 0.4 cP, 0.5 cP, 0.6 cP, 0.7 cP, 0.8 cP, 0.9 cP, 1 cP, 2 cP, 3 cP, 4 cP, 5 cP, 10 cP, 20 cP, cP, 30 cP, 35 cP, 40 cP, 50 cP, 60 cP, 70 cP, 80 cP, 90 cP, 100 cP, 150 cP, 200 cP, 250 cP, 300 cP, 350 cP, 400 cP, 450 cP, 500 cP, 550 cP, 600 cP, 650 cP, 700 cP, 750 cP, 800 cP, 850 cP, 900 cP, 950 cP, 1000 cP, 1500 cP, 2000 cP, 2500 cP, 3000 cP, 3500 cP, 4000 cP, 4500 cP, 5000 cP, or 10000 cP, as measured at a shear rate of at least about 1,000 s⁻¹.
71. The pharmaceutical composition of paragraph 70, wherein the viscosity of the pharmaceutical composition is measured at a shear rate of at least about 1,000 s⁻¹, 2,000 s⁻¹, 3,000 s⁻¹, 4,000 s⁻¹, 5,000 s⁻¹, 6,000 s⁻¹, 7,000 s⁻¹, 8,000 s⁻¹, 9,000 s⁻¹, 10,000 s⁻¹, 15,000 s⁻¹, s⁻¹, or 30,000 s⁻¹.
72. The pharmaceutical composition of any one of paragraphs 1-71, wherein the viscosity of the pharmaceutical composition is about or at most about 35 cP as measured at a shear rate of about 5,000 s⁻¹.
73. The pharmaceutical composition of any one of paragraphs 1-71, wherein the viscosity of the pharmaceutical composition is about or at most about 25 cP as measured at a shear rate of about 10,000 s⁻¹.
74. The pharmaceutical composition of any one of paragraphs 1-71, wherein the viscosity of the pharmaceutical composition is about or at least about 500 cP as measured at a shear rate of at most about 1 s⁻¹.
75. The pharmaceutical composition of any one of paragraphs 1-71, wherein the viscosity of the pharmaceutical composition is about or at least about 1500 cP as measured at a shear rate of at most about 1 s⁻¹.
76. The pharmaceutical composition of any one of paragraphs 1-71, wherein the viscosity of the pharmaceutical composition is about or at most about 362 cP as measured at a shear rate of at least about 1000 s⁻¹.
77. The pharmaceutical composition of any one of paragraphs 3-76, wherein the viscosity of the reference pharmaceutical composition is about or at most about 0.1 cP, 0.2 cP, cP, 0.4 cP, 0.5 cP, 0.6 cP, 0.7 cP, 0.8 cP, 0.9 cP, 1 cP, 1.1 cP, 1.2 cP, 1.3 cP, 1.4 cP, 1.5 cP, 1.6 cP, 1.7 cP, 1.8 cP, 1.9 cP, 2 cP, 2.1 cP, 2.2 cP, 2.3 cP, 2.4 cP, 2.5 cP, 2.6 cP, 2.7 cP, 2.8 cP, 2.9 cP, 3 cP, 3.1 cP, 3.2 cP, 3.3 cP, 3.4 cP, 3.5 cP, 3.6 cP, 3.7 cP, 3.8 cP, 3.9 cP, 4 cP, 4.1 cP, 4.2 cP, 4.3 cP, 4.4 cP, 4.5 cP, 4.6 cP, 4.7 cP, 4.8 cP, 4.9 cP, or 5 cP as measured at a shear rate of at least about 1000 s⁻¹.
78. The pharmaceutical composition of any one of paragraphs 3-77, wherein the viscosity of the reference pharmaceutical composition is about or at most about 0.1 cP, 0.2 cP, cP, 0.4 cP, 0.5 cP, 0.6 cP, 0.7 cP, 0.8 cP, 0.9 cP, 1 cP, 1.1 cP, 1.2 cP, 1.3 cP, 1.4 cP, 1.5 cP, 1.6 cP, 1.7 cP, 1.8 cP, 1.9 cP, 2 cP, 2.1 cP, 2.2 cP, 2.3 cP, 2.4 cP, 2.5 cP, 2.6 cP, 2.7 cP, 2.8 cP, 2.9 cP, 3 cP, 3.1 cP, 3.2 cP, 3.3 cP, 3.4 cP, 3.5 cP, 3.6 cP, 3.7 cP, 3.8 cP, 3.9 cP, 4 cP, 4.1 cP, 4.2 cP, 4.3 cP, 4.4 cP, 4.5 cP, 4.6 cP, 4.7 cP, 4.8 cP, 4.9 cP, or 5 cP as measured at a shear rate of at most about 1 s⁻¹.
79. The pharmaceutical composition of any one of paragraphs 1-78, wherein the viscosity of the pharmaceutical composition is about 0.5 cP to about 400 cP as measured at a shear rate of at least about 1000 s⁻¹.
80. The pharmaceutical composition of any one of paragraphs 1-79, wherein the pharmaceutical composition comprises modified Dulbecco's phosphate-buffered saline solution, and optionally a surfactant.
81. The pharmaceutical composition of any one of paragraphs 1-80, wherein the pharmaceutical composition comprises potassium chloride, potassium phosphate monobasic, sodium chloride, sodium phosphate dibasic anyhydrous, sucrose, and optionally one or more surfactants selected from poloxamer 188, polysorbate 20, and polysorbate 80.
82. The pharmaceutical composition of any one of paragraphs 1-81, wherein the pharmaceutical composition comprises 0.2 mg/mL potassium chloride, 0.2 mg/mL potassium phosphate monobasic, 5.84 mg/mL sodium chloride, 1.15 mg/mL sodium phosphate dibasic anyhydrous, 40.0 mg/mL (4% w/v) sucrose, and optionally a surfactant.
83. The pharmaceutical composition of any one of paragraphs 1-82, wherein the pharmaceutical composition comprises potassium chloride, potassium phosphate monobasic, sodium chloride, sodium phosphate dibasic anyhydrous, sucrose, optionally one or more surfactants selected from poloxamer 188, polysorbate 20, and polysorbate 80, and optionally one or more viscosity modifiers selected from CMC high viscosity grade, CMC medium viscosity grade, CMC low viscosity grade, hydroxypropyl methylcellulose (HPMC), hydroxyethyl cellulose (HES), and poloxamer 407.
84. The pharmaceutical composition of any one of paragraphs 1-83, wherein the pharmaceutical composition comprises potassium chloride, potassium phosphate monobasic, sodium chloride, sodium phosphate dibasic anyhydrous, sucrose, optionally one or more surfactants selected from poloxamer 188, polysorbate 20, and polysorbate 80, and optionally one or more viscosity modifiers selected from 0.5% CMC high viscosity grade, 1% CMC high viscosity grade, 0.5% CMC medium viscosity grade, CMC low viscosity grade, 0.5% hydroxypropyl methylcellulose (HPMC), 0.2% HPMC, 2% hydroxyethyl cellulose (HES), and 15% poloxamer 407.
85. The pharmaceutical composition of any one of paragraphs 1-84, wherein the pharmaceutical composition comprises potassium chloride, potassium phosphate monobasic, sodium chloride, sodium phosphate dibasic anyhydrous, sucrose, one or more surfactants selected from poloxamer 188, polysorbate 20, and polysorbate 80, and one or more polysaccharides selected from CMC, HPMC, and HES.
86. The pharmaceutical composition of any one of paragraphs 1-85, wherein the pharmaceutical composition comprises 0.2 mg/mL potassium chloride, 0.2 mg/mL potassium phosphate monobasic, 5.84 mg/mL sodium chloride, 1.15 mg/mL sodium phosphate dibasic anyhydrous, 40.0 mg/mL (4% w/v) sucrose, 0.001% (0.01 mg/mL) poloxamer 188 and 1% carboxymethyl cellulose (CMC) high viscosity grade.
87. The pharmaceutical composition of any one of paragraphs 1-86, wherein the pharmaceutical composition is stored at about room temperature, 20° C., 4° C., or −80° C.
88. The pharmaceutical composition of any one of paragraphs 1-87, wherein the pharmaceutical composition is stored prior to administration to a human subject.

### 3. BRIEF DESCRIPTION OF THE DRAWINGS

FIGS. 1A-1C. Illustration of the spreading area of a blue dye or a fluorescent dye in eyes ex vivo after suprachoroidal administration of solutions having different viscosity values. FIG. 1A shows the spreading area of a blue dye after suprachoroidal administration of a water solution containing blue dye. FIG. 1B shows the spreading area of a blue dye after suprachoroidal administration of a 1% CMC medium solution containing blue dye. FIG. 1C shows the spreading area of a fluorescent dye after suprachoroidal administration of a 1% CMC medium solution containing fluorescent dye.

FIGS. 2A-2B. Graphs showing the pressure obtained when injecting solutions having different viscosity values, in the SCS. FIG. 2A depicts a graph showing the pressure obtained when a water solution having viscosity of about 1 cP was injected in the SCS. FIG. 2B depicts a graph showing the pressure obtained when a 2% hypromellose solution having viscosity of about 4000 cP was injected in the SCS.

FIG. 3. Graph showing the calculated pressure values (PSI) for different solutions having varying viscosity values (mPas) were injected in the SCS using a 30 gauge needle, at different rates of injection.

FIGS. 4A-4C. Graphs showing calculated injection pressure as a function of viscosity for different 30 gauge and 29 gauge needles. The graphs are scaled to a limit of 100 PSI (FIG. 4A), 65 PSI (FIG. 4B), or 45 PSI (FIG. 4C).

FIG. 5. Graph showing the calculated pressure values (PSI) for different solutions having varying viscosity values (mPas) were injected in the SCS using needles with different gauge sizes: 30 gauge (GA), 30 GA STW, and 29 GA STW needles.

FIG. 6. Graph showing diffusion data obtained for solutions having different viscosity values. Diffusion data was obtained for six solutions containing AAV (e.g., a recombinant adeno-associated virus (AAV) vector comprising an expression cassette encoding a transgene) on the initial day (TO) and after four days at 37° C. The solutions included DPBS with sucrose (control), 0.5% carboxymethyl cellulose (CMC) medium, 0.5% hydroxypropyl methylcellulose (HPMC), 0.2% HPMC, 2% hydroxyethyl cellulose (HES), and 1% CMC low.

FIG. 7. Graph showing the percentage of free DNA obtained for solutions having different viscosity values. Percentage of free DNA was obtained for six solutions containing AAV (e.g., a recombinant adeno-associated virus (AAV) vector comprising an expression cassette encoding a transgene) on the initial day (TO) and after four days at 37° C. The solutions were DPBS with sucrose (control), 0.5% carboxymethyl cellulose (CMC) medium, 0.5% hydroxypropyl methylcellulose (HPMC), 0.2% HPMC, 2% hydroxyethyl cellulose (HES), or 1% CMC low.

FIG. 8. Graph showing DLS thermal ramping (DLS-melt) obtained for solutions having different viscosity values. The solutions included DPBS with sucrose (control), 0.5% carboxymethyl cellulose (CMC) medium, 0.5% hydroxypropyl methylcellulose (HPMC), 0.2% HPMC, 2% hydroxyethyl cellulose (HES), and 1% CMC low.

FIG. 9. Graph showing differential scanning fluorimetry thermal ramp data for solutions having different viscosity values. From top to bottom (S-0C0V to S-0C12), the solutions included DPBS with sucrose (control), 0.5% carboxymethyl cellulose medium (CMC), hydroxypropyl methylcellulose (HPMC), 0.2% HPMC, 2% hydroxyethyl cellulose (HES), and 1% CMC low, 15% poloxamer 407, and 0.5% carboxymethyl cellulose high. Top panel: raw melting curve signal. Middle panel: derivative of data to identify the peak. Bottom panel: light scattering data to indicate either aggregation or gel formation. An increase in light scattering due to a hazy gel formation was observed at about 55° C. for the two hypromellose formulations. The melting temperature onset and midpoints shown by vertical lines in the top panel and the peak in the middle panel were similar for all the formulations, demonstrating that the capsids have similar thermal stability in the different formulations.

FIG. 10. Viscosity (Pas) versus shear rate for 1% CMC high viscosity grade formulation at 20° C.

FIG. 11. Injection pressure into an enucleated porcine eye versus concentration of preparations of CMC medium viscosity grade.

FIG. 12. Injection pressure into an enucleated porcine eye versus concentration of preparations of CMC high viscosity grade.

FIG. 13. Example preparation of clinical drug product with autoclave sterilization.

FIG. 14. Injection pressure measurements for 1% carboxymethylcellulose formulation using a Clearside device and a 30 gauge needle (160 μm needle).

FIG. 15. Differential Scanning Fluorometry Profiles of Control (S-0DGN) and 1% carboxymethylcellulose formulations (S-0DGR).

### 4. DETAILED DESCRIPTION OF THE INVENTION

Provided herein are pharmaceutical compositions comprising recombinant adeno-associated virus (AAV) vector comprising an expression cassette encoding a transgene suitable for administration to a suprachoroidal space (SCS) of an eye of a subject. The subject can be a subject diagnosed with one of more diseases described in Section 4.5. The AAV vectors are described in Section 4.4 and dosages of such vectors are described in Section 4.3. In some embodiments, pharmaceutical compositions provided in Section 4.1 are formulated such that they have one or more functional properties described in Section 4.2. In certain embodiments, the pharmaceutical composition provided herein has various advantages, for example, increased or slower clearance time (Section 4.2.1); decreased circumferential spread (Section 4.2.2); increased SCS thickness (Section 4.2.3); decreased vasodilation and/or vascular leakage (Section 4.2.4); increased AAV level and increased rate of transduction at site of injection (Section 4.2.5);

and increased concentration of the transgene after the pharmaceutical composition is administered in the SCS. Without being bound by theory, the functional properties can be achieved using high viscosity formulations as disclosed in Section 4.1. Also provided herein are assays that may be used in related studies (Section 4.6).

4.1 Formulation of Pharmaceutical Composition

The disclosure provides a pharmaceutical composition suitable for suprachoroidal administration comprising a recombinant adeno-associated virus (AAV) vector comprising an expression cassette encoding a transgene. In some embodiments, several pharmaceutical compositions (e.g., liquid formulation) having different viscosity values are used to administer an AAV encoding a transgene.

In some embodiments, the pharmaceutical composition is more viscous than a comparable pharmaceutical composition (a reference pharmaceutical composition). In some embodiments, the pharmaceutical composition and the reference pharmaceutical composition comprise a recombinant adeno-associated virus (AAV) vector comprising an expression cassette encoding a transgene. In some embodiments, the pharmaceutical composition and the reference pharmaceutical composition have the same vector genome concentration. In some embodiments, the pharmaceutical composition and a reference pharmaceutical composition have the same amount of genome copies. In some embodiments, the pharmaceutical composition has a viscosity value that is higher than the viscosity of water. In some embodiments, the pharmaceutical composition has a viscosity value that is higher than the viscosity of a control. In some embodiments, the pharmaceutical composition has a viscosity value that is higher than the viscosity of a solution normally used for subretinal injection. In some embodiments, the pharmaceutical composition has a viscosity value that is higher than the viscosity of PBS or dPBS. In some embodiments, the pharmaceutical composition has a viscosity value that is higher than the viscosity of Hank's Balanced Salt Solution (HBSS). In some embodiments, the reference pharmaceutical composition has lower viscosity than the pharmaceutical composition. In some embodiments, the reference pharmaceutical composition has the same or similar viscosity than the pharmaceutical composition. In some embodiments, the reference pharmaceutical composition is a control solution (e.g., PBS, water, or HBSS). In some embodiments, the reference pharmaceutical composition comprises sucrose. In some embodiments, the reference pharmaceutical composition is a pharmaceutical composition commonly used for AAV subretinal injection.

In some embodiments, the pharmaceutical composition is characterized by an increase (large increase) in low shear viscosity (e.g., measured at or less than 1 s⁻¹, or an extrapolated zero-rate viscosity of up to 10,000 cP). In some embodiments, the pharmaceutical composition is characterized by an increase (small increase) in high shear viscosity (e.g., defined as a shear rate of about 1000/s to about 5000/s, or extrapolated to 10,000/s or 20,000/s, of less than about 500, 400, 300, 200, 100, 90, 80, 70, 60, 50, 40, 30, 20, or 10 cP). In some embodiments, the viscosity of the pharmaceutical composition is about, at most about, or at least about 5 cP, 10 cP, 20 cP, 25 cP, 30 cP, 35 cP, 40 cP, 50 cP, 60 cP, 70 cP, 80 cP, 90 cP, 100 cP, 110 cP, 120 cP, 125 cP, 130 cP, 135 cP, 140 cP, 150 cP, 160 cP, 170 cP, 180 cP, 190 cP, 200 cP, 210 cP, 220 cP, 225 cP, 230 cP, 235 cP, 240 cP, 250 cP, 260 cP, 270 cP, 280 cP, 290 cP, 300 cP, 310 cP, 320 cP, 325 cP, 330 cP, 335 cP, 340 cP, 350 cP, 360 cP, 370 cP, 380 cP, 390 cP, 400 cP, 410 cP, 420 cP, 425 cP, 430 cP, 435 cP, 440 cP, 450 cP, 460 cP, 470 cP, 480 cP, 490 cP, 500 cP, 550 cP, 600 cP, 650 cP, 700 cP, 750 cP, 800 cP, 850 cP, 900 cP, 950 cP, 1000 cP, 1500 cP, 2000 cP, 2500 cP, 3000 cP, 3500 cP, 4000 cP, 4500 cP, 5000 cP, or 10000 cP, as measured at a shear rate of at least about 1,000 s⁻¹. In some embodiments, the shear rate is of at least about 1,000 s⁻¹, 2,000 s⁻¹, 3,000 s⁻¹, 4,000 s⁻¹, 5,000 s⁻¹, 6,000 s⁻¹, 7,000 s⁻¹, 8,000 s⁻¹, 9,000 s⁻¹, 10,000 s⁻¹, 15,000 s⁻¹, 20,000 s⁻¹, or 30,000 s⁻¹. In some embodiments, the viscosity of the pharmaceutical composition is about 0.1 cP to about 2 cP, about 0.1 cP to about 5 cp, about 0.1 cP to about 10 cP, about 0.1 cP to about 1.5 cP, about 5 cP to about 150 cP, about 5 cP to about 500 cP, about 5 cP to about 300 cP, about 20 cP to about 40 cP, about 15 cP to about 200 cP, about 15 cP to about 50 cP, about 20 cP to about 200 cP, about 20 cP to about 400 cP, about 30 cP to about 40 cP, about 30 cP to about 500 cP, about 30 cP to about 1000 cP, about 20 cP to about 1000 cP as measured at a shear rate of at least about 1,000 s⁻¹ (e.g., 1,000 s⁻¹, 2,000 s⁻¹, 5,000 s⁻¹, 10,000 s⁻¹, or 20,000 s⁻¹). In some embodiments, the viscosity of the pharmaceutical composition is about or at most about 34 cP as measured at a shear rate of about 5,000 s⁻¹. In some embodiments, the viscosity of the pharmaceutical composition is about or at most about 24 cP as measured at or extrapolated to a shear rate of about 10,000 s⁻¹. In some embodiments, the pharmaceutical composition has a viscosity of about or at most about 365 cP at a shear rate of about or more than about 2,000 s⁻¹ (e.g., about or less than about 362 cP at a shear rate of about or more than about 2,100 s⁻¹). In some embodiments, the pharmaceutical composition has a viscosity of about or at most about 35 cP at a shear rate of about or more than about 10,000 s⁻¹ (e.g., about or less than about 34 cP at a shear rate of about 16,000 s⁻¹). In some embodiments, suprachoroidal administration of the pharmaceutical composition is at high shear (e.g., shear rate of at least about 1,000 s⁻¹, or from about 2,000 s⁻¹ to about 20,000 s⁻¹). In some embodiments, the pharmaceutical composition has a viscosity of about or less than about 105 cP at a shear rate of at least about 1,000 s⁻¹ (e.g., about 5,000 s⁻¹), and optionally results in a pressure of about or less than about 43 PSI. In some embodiments, the pharmaceutical composition has a viscosity of about or less than about 365 cP at a shear rate of at least about 1,000 s⁻¹ (e.g., about 2,100 s⁻¹), and optionally results in a pressure of about or less than about 43 PSI. In some embodiments, the pharmaceutical composition has a viscosity of about or less than about 121 cP at a shear rate of at least about 1,000 s⁻¹ (e.g., about 6,300 s⁻¹), and optionally results in a pressure of about or less than about 43 PSI. In some embodiments, the pharmaceutical composition has a viscosity of about or less than about 35 cP at a shear rate of at least about 1,000 s⁻¹ (e.g., about 16,000 s⁻¹), and optionally results in a pressure of about or less than about 43 PSI. In some embodiments, the pharmaceutical composition has a viscosity of about or more than about 500 cP at a shear rate of at least about 1 s⁻¹. In some embodiments, the pharmaceutical composition has a viscosity of about or at least about 1500 cP at a shear rate of at most about 1 s⁻¹. In some embodiments, the pharmaceutical composition has a viscosity of about or at most about 362 cP at a shear rate of at least about 1000 s⁻¹. In some embodiments, the pharmaceutical composition has a viscosity of between about 0.1 cP to about 400 cP at a shear rate of at least about 1,000 s⁻¹. In some embodiments, the reference pharmaceutical composition has a viscosity of about 0.5 cP to about cP at a shear rate of at least about 1 s⁻¹. In some embodiments, the reference pharmaceutical composition has a viscosity of about 0.5 cP to about 10 cP at a shear rate of at least about 1,000 s⁻¹. In some embodiments, the reference pharmaceutical composition is not shear thinning or is slightly shear thinning. In some embodiments, the pharmaceutical composition is shear thinning.

In some embodiments, the pharmaceutical composition has viscosity of about, at least about, or at most about 2 cP, 3 cP, 4 cP, 5 cP, 10 cP, 15 cP, 20 cP, 25 cP, 30 cP, 35 cP, 40 cP, 45 cP, 50 cP, 60 cP, 70 cP, 80 cP, 90 cP, 100 cP, 150 cP, 200 cP, 250 cP, 300 cP, 350 cP, 400 cP, 450 cP, 500 cP, 550 cP, 600 cP, 650 cP, 700 cP, 800 cP, 900 cP, 1000 cP, 1,500 cP, 2,000 cP, 3,000 cP, 4,000 cP, 5,000 cP, 6,000 cP, 7,000 cP, 8,000 c, 9,000 cP, 10,000 cP, 12,000 cP, or cP e.g., at zero, 0.001, 0.01, 0.1 or 1 s⁻¹ shear rate or at a shear rate of about or at most about 1 s⁻¹. In some embodiments, the shear rate is about or less than about 100 s⁻¹, 50 s⁻¹, 10 s⁻¹, 1 s⁻¹, 0.1 s⁻¹, 0.01 s⁻¹, 0.001 s⁻¹, or 0.0001 s⁻¹. In some embodiments, the viscosity of the pharmaceutical composition or the reference pharmaceutical composition is any viscosity disclosed herein at a shear rate of e.g., about or less than about 100 s⁻¹, 50 s⁻¹, 10 s⁻¹, 1 s⁻¹, 0.01 s⁻¹, 0.001 s⁻¹, or 0.0001 s⁻¹. In some embodiments, the pharmaceutical composition or the reference pharmaceutical composition undergoes shear thinning during injection.

In some embodiments, the reference pharmaceutical composition has a viscosity of about or at most about 0.1 cP, 0.2 cP, 0.3 cP, 0.4 cP, 0.5 cP, 0.6 cP, 0.7 cP, 0.8 cP, 0.9 cP, 1 cP, 1.1 cP, 1.2 cP, 1.3 cP, 1.4 cP, 1.5 cP, 1.6 cP, 1.7 cP, 1.8 cP, 1.9 cP, 2 cP, 2.1 cP, 2.2 cP, 2.3 cP, 2.4 cP, 2.5 cP, 2.6 cP, 2.7 cP, 2.8 cP, 2.9 cP, 3 cP, 3.1 cP, 3.2 cP, 3.3 cP, 3.4 cP, 3.5 cP, 3.6 cP, 3.7 cP, 3.8 cP, 3.9 cP, 4 cP, 4.1 cP, 4.2 cP, 4.3 cP, 4.4 cP, 4.5 cP, 4.6 cP, 4.7 cP, 4.8 cP, 4.9 cP, or 5 cP as measured at a shear rate of at least about 1000 s⁻¹. In some embodiments, the reference pharmaceutical composition has a viscosity of about or at most about 0.1 cP, 0.2 cP, cP, 0.4 cP, 0.5 cP, 0.6 cP, 0.7 cP, 0.8 cP, 0.9 cP, 1 cP, 1.1 cP, 1.2 cP, 1.3 cP, 1.4 cP, 1.5 cP, 1.6 cP, 1.7 cP, 1.8 cP, 1.9 cP, 2 cP, 2.1 cP, 2.2 cP, 2.3 cP, 2.4 cP, 2.5 cP, 2.6 cP, 2.7 cP, 2.8 cP, 2.9 cP, 3 cP, 3.1 cP, 3.2 cP, 3.3 cP, 3.4 cP, 3.5 cP, 3.6 cP, 3.7 cP, 3.8 cP, 3.9 cP, 4 cP, 4.1 cP, 4.2 cP, 4.3 cP, 4.4 cP, 4.5 cP, 4.6 cP, 4.7 cP, 4.8 cP, 4.9 cP, or 5 cP as measured at a shear rate of at most about 1 s⁻¹.

In some embodiments, the pharmaceutical composition (e.g., liquid formulation) has a viscosity (low shear viscosity e.g., about or at most about 1 s⁻¹ or extrapolated zero-rate) or shear viscosity that is about or at least about 5 cP, about or at least about 10 cP, about or at least about 15 cP, about or at least about 20 cP, about or at least about 25 cP, about or at least about 30 cP, about or at least about 35 cP, about or at least about 40 cP, about or at least about 45 cP, about or at least about 50 cP, about or at least about 60 cP, about or at least about 70 cP, about or at least about 80 cP, about or at least about 90 cP, 100 cP, about or at least about 115 cP, about or at least about 120 cP, about or at least about 125 cP, about or at least about 130 cP, about or at least about 135 cP, about or at least about 140 cP, about or at least about 145 cP, about or at least about 150 cP, about or at least about 160 cP, about or at least about 170 cP, about or at least about 180 cP, about or at least about 190 cP, about or at least about 200 cP, about or at least about 300 cP, about or at least about 400 cP, about or at least about 500 cP, about or at least about 600 cP, about or at least about 700 cP, about or at least about 800 cP, about or at least about 900 cP, about or at least about 1000 cP, about or at least about 1500 cP, about or at least about 2000 cP, about or at least about 2500 cP, about or at least about 3000 cP, about or at least about 3500 cP, about or at least about 4000 cP, about or at least about 4500 cP, about or at least about 5000 cP, about or at least about 5500 cP, about or at least about 6000 cP, about or at least about 6500 cP, about or at least about 7000 cP, about or at least about 7500 cP, about or at least about 8000 cP, about or at least about 9000 cP, about or at least about 10000 cP, about or at least about 1×10³ cP, about or at least about 3×10³ cP, about or at least about 1×10⁴ cP, about or at least about 3×10⁴ cP, about or at least about 1×10⁵ cP, about or at least about 1.7×10⁵ cP, about or at least about 3×10⁵ cP, about or at least about 1×10⁶ cP, about or at least about 3×10⁶ cP, about or at least about 1×10⁷ cP, about or at least about 3×10⁷ cP, about or at least about 1×10⁸ cP, or about or at least about 3×10⁸ cP (e.g., as measured at a shear rate of at most about 1 s⁻¹). In some embodiments, the viscosity is between about 25 cP to about 1×10⁶ cP, between about 25 cP to about 1×10⁴ cP, between about 25 cP to about 5,000 cP, between about 25 cP to about 1×10³ cP, between about 100 cP to about 1×10⁶ cP, between about 100 cP to about 1×10⁴ cP, between about 100 cP to about 5,000 cP, between about 100 cP to about 1×10³ cP. In some embodiments, the viscosity is between about 25 cP to about 3×10⁶ cP, between about 10 cP to about 3×10⁸ cP, between about 50 cP to about 5000 cP, between about 10 cP to about 15000 cP, between about 25 cP to about 1500 cP, between about 50 cP to about 1500 cP, between about 25 cP to about 3×10⁴ cP. In some embodiments, the pharmaceutical composition has a viscosity that is at least between about 25 cP to about 3×10⁶ cP, at least between about 10 cP to about 3×10⁸ cP, at least between about 50 cP to about 5000 cP, at least between about 10 cP to about 15000 cP, at least between about 25 cP to about 1500 cP, at least between about 50 cP to about 1500 cP, or at least between about 25 cP to about 3×10⁴ cP. In some embodiments, a comparable pharmaceutical composition, or a reference pharmaceutical composition, or a control has a viscosity of about or at most about 0.1 cP, about or at most about 0.2 cP, about or at most about 0.3 cP, about or at most about 0.4 cP, about or at most about 0.5 cP, about or at most about cP, about or at most about 0.7 cP, about or at most about 0.8 cP, about or at most about 0.9 cP, about or at most about 1 cP, about or at most about 1.1 cP, about or at most about 1.2 cP, about or at most about 1.3 cP, about or at most about 1.4 cP, about or at most about 1.5 cP, about or at most about 1.6 cP, about or at most about 1.7 cP, about or at most about 1.8 cP, about or at most about 1.9 cP, about or at most about 2 cP, about or at most about 3 cP, about or at most about 4 cP, about or at most about 5 cP, about or at most about 6 cP, about or at most about 7 cP, about or at most about 8 cP, about or at most about 9 cP, about or at most about 10 cP, about or at most about 15 cP, about or at most about 20 cP, about or at most about 25 cP, about or at most about 30 cP, about or at most about 35 cP, about or at most about 40 cP, about or at most about 45 cP, about or at most about 50 cP, about or at most about 55 cP, about or at most about 60 cP, about or at most about 65 cP, about or at most about 70 cP, about or at most about 75 cP, about or at most about 80 cP, about or at most about 85 cP, about or at most about 90 cP, about or at most about 95 cP, about or at most about 100 cP, about or at most about 200 cP, about or at most about 300 cP, about or at most about 400 cP, or about or at most about 500 cP (e.g., as measured at a shear rate of at most about 1s⁻¹). In some embodiments, a comparable pharmaceutical composition, or a reference pharmaceutical composition, or a control has a viscosity of about or at most about 0.1 cP, 0.2 cP, 0.3 cP, 0.4 cP, 0.5 cP, 1 cP, 1.3 cP, 1.5 cP, 2 cP, 3 cP, 5 cP, or 10 cP (e.g., at a shear rate of at least about 1000 s⁻¹). In some embodiments, a comparable pharmaceutical composition, or a reference pharmaceutical composition, or a control has a viscosity of about or at most about 2 cP (e.g., at a shear rate of at least about 1000 s⁻¹). In some embodiments, a comparable pharmaceutical composition, or a reference pharmaceutical composition, or a control has a viscosity of about or at most about 1.5 cP (e.g., at a shear rate of at least about 1000 s⁻¹). In some embodiments, a comparable pharmaceutical composition, or a reference pharmaceutical composition, or a control has a viscosity of about 1.3 cP (e.g., at a shear rate of at most about 1 s⁻¹). In some embodiments, a comparable pharmaceutical composition, or a reference pharmaceutical composition, or a control has a viscosity of between about 0.1 cP to about 3 cP, about 0.1 cP to about 2 cP, 0.1 cP to about 1.5 cP, 0.1 cP to about 5 cP, 1 cP to about 20 cP, between about 1 cP to about 24 cP, between about 1 cP to about 25 cP, between about 1 cP to about 10 cP, between about 1 cP to about 50 cP, between about 1 cP to about 100 cP, between about 5 cP to about 50 cP, between about 1 cP to about 5 cP, or between about 1 cP to about 200 cP. In some embodiments, a reference pharmaceutical composition has a viscosity of about 1 cP or less than about 1 cP (e.g., at a shear rate of at most about 1 s⁻¹). In some embodiments, a reference pharmaceutical composition has a viscosity of less than about 1 cP (e.g., at a shear rate of at least about 1000 s⁻¹). Because viscosity depends on shear rate, the “low shear or zero-rate viscosity” of the pharmaceutical composition (e.g., liquid formulation) is the viscosity at any point between a shear rate of 0.01 s⁻¹ to 1 s⁻¹. In some embodiments, the unit for viscosity can be defined as cP or mPas. In some cases, cP and mPas are used interchangeably.

In some embodiments, the viscosity (or shear viscosity at zero or 1 s⁻¹) of the pharmaceutical composition (e.g., liquid formulation) is at least about 10 cP, or at least about 100 cP, or at least about 1000 cP, or at least about 10,000 cP, or at least about 70,000 cP, or up to about 200,000 cP, or up to about 250,000 cP, or up to about 300,000 cP or more. In some embodiments, a shear rate is a shear rate of 0.1/second. In some embodiments, a formulation is characterized by a zero shear viscosity of at least 300,000 mPas. In some embodiments, the pharmaceutical composition is characterized by a viscosity of not more than about 400 mPas at 1000 s⁻¹ shear rate. In some embodiments, a pharmaceutical composition can have a viscosity of between about 130,000 cP and about 300,000 cP at a shear rate of about 0.1/second at about 25° C. In some embodiments, a viscosity at zero or 1 s⁻¹ is at least about 2, 3, 5, 10, or 20 (or more than 20) times less than the viscosity at a shear rate of at least 1000 s⁻¹ (e.g., 1,000 s⁻¹, 2,000 s⁻¹, s⁻¹, 10,000 s⁻¹, or 20,000 s⁻¹). In some embodiments, a viscosity at 100 s⁻¹ is at least about 2, 3, 5, 10 or even 20 or more times less than at a shear rate of 5 s⁻¹. In some embodiments, the stress at which shear-thinning starts is known as a yield stress. In some embodiments, a certain shear stress (force) is required before the pharmaceutical composition starts to flow readily. This critical shear stress is often called the yield stress. The yield stress can be determined from a steady state flow curve measured with a stress controlled rheometer. When the viscosity is plotted as a function of applied shear stress, a dramatic decrease in viscosity is seen after exceeding the critical shear stress. In some embodiments, the yield stress is about, at least about, or at most about 0.0001 Pa, 0.0005 Pa, 0.001 Pa, 0.005 Pa, 0.01 Pa, 0.05 Pa, 0.1 Pa, 0.5 Pa, 1 Pa, 2 Pa, 3 Pa, 5 Pa, 10 Pa, 15 Pa, 20 Pa, 25 Pa, 30 Pa, 35 Pa, 40 Pa, 45 Pa, 50 Pa, 55 Pa, 60 Pa, 65 Pa, 70 Pa, 75 Pa, 80 Pa, 85 Pa, 90 Pa, 100 Pa, 110 Pa, 120 Pa, 130 Pa, 140 Pa, 150 Pa, 200 Pa, 250 Pa, 300 Pa, 350 Pa, 400 PA, 450 Pa, 500 Pa, or more than 500 Pa.

In some embodiments, a relatively high viscosity pharmaceutical composition remains in the SCS (or in the eye) for a longer period of time after injection (measured at different time points) as compared to low viscosity formulations, or formulations having lower viscosity. In some embodiments, a higher viscosity pharmaceutical composition expands the SCS or the thickness at the site of injection (e.g., as compared to low viscosity formulations, or formulations having lower viscosity) (see Section 4.2.3).

In some embodiments, the pharmaceutical composition (e.g., liquid formulation) has a viscosity sufficient to expand at least a portion of the site of injection (e.g. SCS) to a thickness of at least 500 μm or about 500 μm to about 3 mm, for at least two hours after administration. In some embodiments, the viscosity of the pharmaceutical composition (e.g., liquid formulation) is sufficient to expand the site of injection (e.g. SCS) to a thickness of about 750 μm to about 2.8 mm, about 750 μm to about 2.5 mm, about 750 μm to about 2 mm, or about 1 mm to about 2 mm. In some embodiments, the viscosity of the pharmaceutical composition (e.g., liquid formulation) is sufficient to expand the site of injection (e.g. SCS) to a thickness of about 500 μm to about 3.0 mm for at least two hours, at least three hours, at least four hours, at least five hours, at least six hours, at least seven hours, at least eight hours, at least ten hours, at least twelve hours, at least eighteen hours, at least twenty-four hours, at least two days, at least three days, at least five days, at least ten days, at least twenty-one days, at least one month, at least six weeks, at least two months, at least three months, at least 4 months, at least 5 months, at least 6 months, at least 9 months, at least one year, at least three years, or at least five years after the administration. In some embodiments, the viscosity of the pharmaceutical composition (e.g., liquid formulation) is sufficient to expand the site of injection (e.g. SCS) to a thickness of about 1 mm to about 3 mm for at least two hours, at least three hours, at least four hours, at least five hours, at least six hours, at least seven hours, at least eight hours, at least ten hours, at least twelve hours, at least eighteen hours, or at least twenty-four hours after administration. In some embodiments, the viscosity of the pharmaceutical composition (e.g., liquid formulation) is sufficient to expand the site of injection (e.g. SCS) to a thickness of about 1 mm to about 2 mm for at least two hours, at least three hours, at least four hours, at least five hours, at least six hours, at least seven hours, at least eight hours, at least ten hours, at least twelve hours, at least eighteen hours, at least twenty-four hours, at least two days, at least three days, at least five days, at least ten days, at least twenty-one days, at least one month, at least six weeks, at least two months, at least three months, at least 4 months, at least 5 months, at least 6 months, at least 9 months, at least one year, at least three years, or at least five years after the administration. In some embodiments, the viscosity of the pharmaceutical composition (e.g., liquid formulation) is sufficient to expand the site of injection (e.g. SCS) to a thickness of about 2 mm to about 3 mm for at least two hours, at least three hours, at least four hours, at least five hours, at least six hours, at least seven hours, at least eight hours, at least ten hours, at least twelve hours, at least eighteen hours, at least twenty-four hours, at least two days, at least three days, at least five days, at least ten days, at least twenty-one days, at least one month, at least six weeks, at least two months, at least three months, at least 4 months, at least 5 months, at least 6 months, at least 9 months, at least one year, at least three years, or at least five years after the administration. In some embodiments, the viscosity of the pharmaceutical composition (e.g., liquid formulation) is sufficient to expand the site of injection (e.g. SCS) to a thickness of about 750 μm to about 2.8 mm, about 750 μm to about 2.5 mm, about 750 μm to about 2 mm, or about 1 mm to about 2 mm for an indefinite period. An indefinite period may be achieved due, at least in part, to the stability of the pharmaceutical composition (e.g., liquid formulation) in the site of injection (e.g. SCS).

In some embodiments, a pharmaceutical composition (e.g., liquid formulation) having a viscosity sufficient to expand the site of injection (e.g. SCS) to a thickness of at least 500 μm, or about 500 μm to about 3 mm, has a viscosity greater than the viscosity of water (i.e., about 1 cP). In some embodiments, a pharmaceutical composition (e.g., liquid formulation) has a viscosity sufficient to expand the site of injection (e.g. SCS) to a thickness of at least about 50 μm, 100 μm, 200 μm, 300 μm, 400 μm, 500 μm, 600 μm, 700 μm, 800 μm, 900 μm, 1000 μm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, or larger than 10 mm. In some embodiments, a reference pharmaceutical composition has a viscosity sufficient to expand the site of injection to a thickness of at most about 1 nm, 5 nm, 10 nm, 25 nm, 50 nm, 100 nm, 200 nm, 300 nm, 400 nm, 500 nm, 600 nm, 700 nm, 800 nm, 900 nm, 1 μm, 5 μm, 10 μm, 15 μm, 20 μm, 25 μm, 30 μm, 35 μm, 40 μm, 50 μm, 100 μm, 200 μm, 300 μm, 400 μm, 500 μm, 600 μm, 700 μm, 800 μm, 900 μm, 1000 μm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, or 10 mm.

In some embodiments, the pharmaceutical composition (e.g., liquid formulation) having a viscosity sufficient to expand the site of injection (e.g. SCS) to a thickness of at least 500 μm, or about 500 μm to about 3 mm, includes a polysaccharide. See, e.g., polysaccharides described in Section 4.1.1.

Also provided herein are methods of treating a disease (e.g., an ocular disease) described in Section 4.5 using the pharmaceutical compositions disclosed herein. In some embodiments, a method of treating an ocular disease includes administering an effective amount of the pharmaceutical composition (e.g., recombinant adeno-associated virus (AAV) vector comprising an expression cassette encoding a transgene) to a subject (e.g., human). In some embodiments, the pharmaceutical composition is administered in the suprachoroidal space (SCS) of an eye of the subject. In some embodiments, the effective amount of the pharmaceutical composition sufficient to elicit a therapeutic response when administered to the SCS is less than the effective amount of the pharmaceutical composition sufficient to elicit a therapeutic response when administered subretinally. In some embodiments, the effective amount of the pharmaceutical composition sufficient to elicit a therapeutic response when administered to the SCS is less than the effective amount of the pharmaceutical composition sufficient to elicit a therapeutic response when administered intravitreously. In some embodiments, the pharmaceutical composition has the same vector genome concentration when administered to the SCS as when administered via subretinal administration or via intravitreous administration. In some embodiments, the pharmaceutical composition has the same amount of genome copies when administered to the SCS as when administered via subretinal administration or via intravitreous administration. In some embodiments, the effective amount of the pharmaceutical composition sufficient to elicit a therapeutic response in a subject is lower as compared to the effective amount of a reference pharmaceutical composition sufficient to elicit a therapeutic response in the subject when administered to the SCS. In some embodiments, the effective amount of the pharmaceutical composition sufficient to elicit a therapeutic response when administered to the SCS is less than the effective amount of a reference pharmaceutical composition sufficient to elicit a therapeutic response when administered subretinally. In some embodiments, the effective amount of the pharmaceutical composition sufficient to elicit a therapeutic response when administered to the SCS is less than the effective amount of a reference pharmaceutical composition sufficient to elicit a therapeutic response when administered intravitreously. In some embodiments, the pharmaceutical composition and the reference pharmaceutical composition have the same vector genome concentration. In some embodiments, the pharmaceutical composition and the reference pharmaceutical composition have the same amount of genome copies. In some embodiments, the pharmaceutical composition has a viscosity that is higher than the viscosity of the reference pharmaceutical composition.

In some embodiments, the pharmaceutical composition is substantially localized near the insertion site (see Section 4.2.1 and Section 4.2.2). In some embodiments, the pharmaceutical composition results in a higher level of transgene expression (concentration) when the pharmaceutical composition is administered in the SCS as compared to when the pharmaceutical composition is administered subretinally or intravitreously (see Section 4.2.6). In some embodiments, the pharmaceutical composition results in a higher level of transgene expression (concentration) when the pharmaceutical composition is administered in the SCS as compared to when a reference pharmaceutical composition is administered subretinally, intravitreously, or in the SCS (see Section 4.2.6). In some embodiments, the pharmaceutical composition results in a higher level of AAV when the pharmaceutical composition is administered in the SCS as compared to when the pharmaceutical composition is administered subretinally or intravitreously (see Section 4.2.5). In some embodiments, the pharmaceutical composition results in a higher level of AAV when the pharmaceutical composition is administered in the SCS as compared to when a reference pharmaceutical composition is administered subretinally, intravitreously, or in the SCS (see Section 4.2.5). In some embodiments, the pharmaceutical composition results in a higher rate of transduction (or rate of infection) at a site of injection when the pharmaceutical composition is administered in the SCS as compared to when the pharmaceutical composition is administered subretinally or intravitreously (see Section 4.2.5). In some embodiments, the pharmaceutical composition results in a higher rate of transduction (or rate of infection) at a site of injection when the pharmaceutical composition is administered in the SCS as compared to when a reference pharmaceutical composition is administered subretinally, intravitreously, or in the SCS (see Section 4.2.5),In some embodiments, the pharmaceutical composition results in reduced vasodilation and/or vascular leakage when the pharmaceutical composition is administered in the SCS as compared to when the pharmaceutical composition is administered subretinally or intravitreously (see Section 4.2.4). In some embodiments, the pharmaceutical composition results in reduced vasodilation and/or vascular leakage when the pharmaceutical composition is administered in the SCS as compared to when a reference pharmaceutical composition is administered subretinally, intravitreously, or in the SCS (see Section 4.2.4). In some embodiments, the reference pharmaceutical composition includes the recombinant adeno-associated virus (AAV) vector comprising the expression cassette encoding the transgene. In some embodiments, the pharmaceutical composition has higher viscosity than the reference pharmaceutical composition. In some embodiments, the pharmaceutical composition and the reference pharmaceutical composition have the same vector genome concentration. In some embodiments, the pharmaceutical composition and the reference pharmaceutical composition have the same amount of genome copies.

4.1.1 Manipulation of Viscosity

In some embodiments, a viscosity-inducing component is present in an amount to increase the viscosity of the pharmaceutical composition (e.g., liquid formulation). In some embodiments, increasing the viscosity of the formulation to values well in excess of the viscosity of water, for example, at least about 100 cP at a shear rate of 0.1/second to 1/second, results in formulations that are highly effective for placement, e.g., injection, into the SCS of an eye of a subject. In some embodiments, the relatively high viscosity of the formulation enhances the ability of such formulations to maintain the therapeutic component (e.g., AAV comprising an expression cassette comprising a transgene) in substantially uniform suspension in the formulation for prolonged periods of time, and can also aid in the storage stability of the formulation.

In some embodiments, a low viscosity pharmaceutical composition (e.g., liquid formulation) is used to administer an AAV encoding a transgene. In some embodiments, a pharmaceutical composition (e.g., liquid formulation) having medium viscosity is used to administer an AAV encoding a transgene. In some embodiments, a high viscosity pharmaceutical composition (e.g., liquid formulation) is used to administer an AAV encoding a transgene. In some embodiments, a pharmaceutical composition (e.g., liquid formulation) having a higher viscosity as compared to a control solution, or as compared to PBS, or as compared to a commonly used pharmaceutical composition (e.g., liquid formulation) for subretinal injection, is used to administer an AAV encoding a transgene. Non-limiting examples of solutions that have low viscosity and that can be used in a pharmaceutical composition of the present disclosure include a solution containing sucrose (e.g., 100 mM NaCl and 4% sucrose, 6% sucrose, or 10% sucrose (viscosity of about 1.3 cP)), PEG3350, dextran 40k, PEG12000, and/or carboxymethyl cellulose sodium salt (viscosity of 10-50 cP; 2% H₂O, 25° C.). Non-limiting examples of solutions that have medium viscosity and that can be used in a pharmaceutical composition of the present disclosure include carboxymethyl cellulose sodium salt (2%=400-800 cP), polyvinyl alcohol 26-88 (4%=25 cP) (i.e., Mowiol®), and/or hydroxyethyl cellulose (viscosity of 100 cP NF) (i.e., hetastarch). Non-limiting examples of solutions that have high or very high viscosity and that can be used in a pharmaceutical composition of the present disclosure include carboxymethyl cellulose sodium salt (high viscosity of 1500-3000 cP) (1% H₂O, 25° C.), hydroxypropyl methylcellulose (hypromellose) (high viscosity of 4000 mPas, Type 2910), and/or polyvinylpyrrolidone (povidone K-90) (M.W. ˜360,000 K-90; very high viscosity).

In some embodiments, the pharmaceutical composition (e.g., liquid formulation) comprises a polysaccharide. In some embodiments, the pharmaceutical composition (e.g., liquid formulation) comprises carboxymethyl cellulose sodium salt. In some embodiments, the pharmaceutical composition (e.g., liquid formulation) comprises carboxymethyl cellulose sodium salt (viscosity of 10-50 cP; 2% H₂O, 25° C.). In some embodiments, the pharmaceutical composition (e.g., liquid formulation) comprises carboxymethyl cellulose sodium salt (2%=400-800 cP). In some embodiments, the pharmaceutical composition (e.g., liquid formulation) comprises hydroxyethyl cellulose (viscosity of 100 cP NF) (i.e., hetastarch). In some embodiments, the pharmaceutical composition (e.g., liquid formulation) comprises carboxymethyl cellulose sodium salt (high viscosity of 1500-3000 cP) (1% H₂O, 25° C.). In some embodiments, the pharmaceutical composition (e.g., liquid formulation) comprises hydroxypropyl methylcellulose (hypromellose) (high viscosity of 4000 mPas, Type 2910).

In some embodiments, the pharmaceutical composition (e.g., liquid formulation) includes a polysaccharide at a concentration of about 0.2% to about 50% by weight, based on the weight of the pharmaceutical composition (e.g., liquid formulation). In some embodiments, the pharmaceutical composition (e.g., liquid formulation) includes a polysaccharide at a concentration of about 0.5% to about 2% by weight, based on the weight of the pharmaceutical composition (e.g., liquid formulation). In some embodiments, the pharmaceutical composition (e.g., liquid formulation) includes a polysaccharide at a concentration of about 0.2% to about 40% by weight, based on the weight of the pharmaceutical composition (e.g., liquid formulation). In some embodiments, the pharmaceutical composition (e.g., liquid formulation) includes a polysaccharide at a concentration of about 0.2% to about 30% by weight, based on the weight of the pharmaceutical composition (e.g., liquid formulation). In some embodiments, the pharmaceutical composition (e.g., liquid formulation) includes a polysaccharide at a concentration of about 0.2% to about 20% by weight, based on the weight of the pharmaceutical composition (e.g., liquid formulation). In some embodiments, the pharmaceutical composition (e.g., liquid formulation) includes a polysaccharide at a concentration of about 0.2% to about 10% by weight, based on the weight of the pharmaceutical composition (e.g., liquid formulation). In some embodiments, the pharmaceutical composition (e.g., liquid formulation) includes a polysaccharide at a concentration of about 0.2% to about 5% by weight, based on the weight of the pharmaceutical composition (e.g., liquid formulation). The polysaccharide can be selected from any biocompatible polysaccharide, such as carboxymethylcellulose, dextran, hyaluronic acid, chondroitin sulfate, or a combination thereof. In some embodiments, a pharmaceutical composition (e.g., liquid formulation) that exhibits non-Newtonian shear thinning behavior is desirable, as the viscosity is lower under high shear during infusion through a needle. The pharmaceutical composition (e.g., liquid formulation) can also include an additive at a concentration sufficient to draw a portion of one or more ocular fluids into the site of injection (e.g. SCS). The drawing of one or more ocular fluids into the site of injection (e.g. SCS) may assist the expansion of the site of injection (e.g. SCS). In some embodiments, the one or more additives include a polysaccharide. In some embodiments, the viscosity-inducing component is present in an amount in a range of about 0.5% or about 1.0% to about 5% or about 10% or about 20% (w/v) of the formulation.

In some embodiments, viscosity inducing agents (e.g. viscosity modifiers) are used to increase the viscosity of the pharmaceutical composition (e.g., liquid formulation). Examples of viscosity-inducing components include, but are not limited to, hyaluronic acid, carbomers, polyacrylic acid, cellulosic derivatives, polycarbophil, polyvinylpyrrolidone, gelatin, dextrin, polysaccharides, polyacrylamide, polyvinyl alcohol, polyvinyl acetate, derivatives thereof and mixtures thereof.

In some embodiments, the pharmaceutical composition includes a polymeric component. Polymeric component includes any polymeric material useful in a body of a mammal, whether derived from a natural source or synthetic. Examples of polymeric materials that can be used in the formulation include carbohydrate based polymers such as methylcellulose, carboxymethylcellulose, hydroxymethylcellulose, hydroxypropylcellulose, hydroxyethylcellulose, ethyl cellulose, dextrin, cyclodextrins, alginate, hyaluronic acid and chitosan, protein based polymers such as gelatin, collagen and glycolproteins, and hydroxy acid polyesters such as bioerodable polylactide-coglycolide (PLGA), polylactic acid (PLA), polyglycolide, polyhydroxybutyric acid, polycaprolactone, polyvalerolactone, polyphosphazene, and polyorthoesters. Polymers can also be crosslinked, blended or used as copolymers in the formulation. Other polymer carriers include albumin, polyanhydrides, polyethylene glycols, polyvinyl polyhydroxyalkyl methacrylates, pyrrolidone and polyvinyl alcohol.

Some examples of non-erodible polymers that can be used in the pharmaceutical composition include silicone, polycarbonates, polyvinyl chlorides, polyamides, polysulfones, polyvinyl acetates, polyurethane, ethylvinyl acetate derivatives, acrylic resins, crosslinked polyvinyl alcohol and crosslinked polyvinylpyrrolidone, polystyrene and cellulose acetate derivatives.

In some embodiments, the molecular weight of the viscosity-inducing component is in a range up to about 2 million Daltons, such as of about 10,000 Daltons or less to about 2 million Daltons or more. In some embodiments, the molecular weight of the viscosity-inducing component is in a range of about 100,000 Daltons or about 200,000 Daltons to about 1 million Daltons or about 1.5 million Daltons. In some embodiments, a viscosity-inducing component is a polymeric hyaluronate component, for example, a metal hyaluronate component, such as alkali metal hyaluronates, alkaline earth metal hyaluronates and mixtures thereof, sodium hyaluronates, and mixtures thereof. In some embodiments, the molecular weight of such hyaluronate component is in a range of about 50,000 Daltons or about 100,000 Daltons to about 1.3 million Daltons or about 2 million Daltons.

4.1.2 Other Components of the Formulation

In some embodiments, the disclosure provides a pharmaceutical composition (e.g., liquid formulation) comprising a recombinant adeno-associated virus (AAV) and at least one of: potassium phosphate monobasic, sodium chloride, sodium phosphate dibasic anhydrous, sucrose, and surfactant. In some embodiments, the pharmaceutical composition (e.g., liquid formulation) does not comprise sucrose.

In some embodiments, the disclosure provides a pharmaceutical composition comprising a recombinant adeno-associated virus (AAV) and at least one of: an ionic salt excipient or buffering agent, sucrose, and surfactant. In some embodiments, the ionic salt excipient or buffering agent can be one or more components from the group consisting of potassium phosphate monobasic, potassium phosphate, sodium chloride, sodium phosphate dibasic anhydrous, sodium phosphate hexahydrate, sodium phosphate monobasic monohydrate, tromethamine, tris(hydroxymethyl)aminomethane hydrochloride (Tris-HCl), amino acid, histidine, histidine hydrochloride (histidine-HCl), sodium succinate, sodium citrate, sodium acetate, and (4-(2-hydroxyethyl)-1-piperazineethanesulfonic acid) (HEPES), sodium sulfate, magnesium sulfate, magnesium chloride 6-hydrate, calcium sulfate, potassium chloride, calcium chloride, and calcium citrate. In some embodiments, the surfactant can be one or more components from the group consisting of poloxamer 188, polysorbate 20, and polysorbate 80. In some embodiments, the disclosure provides a pharmaceutical composition comprising a recombinant adeno-associated virus (AAV) and one or more viscosity modifiers. Examples of viscosity modifiers include, but are not limited to, carboxymethylcellulose (CMC) high viscosity grade, CMC medium viscosity grade, CMC low viscosity grade, hydroxypropyl methylcellulose (HPMC), hydroxyethyl cellulose (HES), and poloxamer 407. In some embodiments, the disclosure provides a pharmaceutical composition comprising a recombinant adeno-associated virus (AAV) and one or more polysaccharides including but not limited to any derivative of cellulose or starch, such as CMC, HPMC, and HES. In some embodiments, the disclosure provides a pharmaceutical composition comprising 0.2 to 15% carboxymethyl cellulose (CMC) high viscosity grade, CMC high viscosity grade, CMC medium viscosity grade, hydroxypropyl methylcellulose (HPMC), HPMC, hydroxyethyl cellulose (HES), CMC low viscosity grade, and/or poloxamer 407. In some embodiments, the disclosure provides a pharmaceutical composition comprising 0.2 to 10% carboxymethyl cellulose (CMC) high viscosity grade, CMC high viscosity grade, CMC medium viscosity grade, hydroxypropyl methylcellulose (HPMC), HPMC, hydroxyethyl cellulose (HES), or CMC low viscosity grade, and 15% poloxamer 407.

In certain embodiments, the pharmaceutical composition has an ionic strength of about 60 mM to about 115 mM. In certain embodiments, the pharmaceutical composition has an ionic strength of about 60 mM to about 100 mM. In certain embodiments, the pharmaceutical composition has an ionic strength of about 65 mM to about 95 mM. In certain embodiments, the pharmaceutical composition has an ionic strength of about 70 mM to about 90 mM. In certain embodiments, the pharmaceutical composition has an ionic strength of about 75 mM to about 85 mM.

In certain embodiments, the pharmaceutical composition has an ionic strength of about 30 mM to about 100 mM. In certain embodiments, the pharmaceutical composition has an ionic strength of about 35 mM to about 95 mM. In certain embodiments, the pharmaceutical composition has an ionic strength of about 40 mM to about 90 mM. In certain embodiments, the pharmaceutical composition has an ionic strength of about 45 mM to about 85 mM. In certain embodiments, the pharmaceutical composition has an ionic strength of about 50 mM to about 80 mM. In certain embodiments, the pharmaceutical composition has an ionic strength of about 55 mM to about 75 mM. In certain embodiments, the pharmaceutical composition has an ionic strength of about 60 mM to about 70 mM.

In certain embodiments, the pharmaceutical composition comprises potassium chloride (e.g., at a concentration of 0.2 g/L). In certain embodiments, the pharmaceutical composition comprises potassium phosphate monobasic (e.g., at a concentration of 0.2 g/L). In certain embodiments, the pharmaceutical composition comprises sodium chloride (e.g., at a concentration of 5.84 g/L). In certain embodiments, the pharmaceutical composition comprises sodium phosphate dibasic anhydrous (e.g., at a concentration of 1.15 g/L). In certain embodiments, the pharmaceutical composition comprises potassium chloride, potassium phosphate monobasic, sodium chloride, and sodium phosphate dibasic anhydrous.

In some embodiments, the reference pharmaceutical composition comprises the same components as the pharmaceutical composition. In some embodiments, the reference pharmaceutical composition comprises the same components as the pharmaceutical composition but has a lower viscosity value than the pharmaceutical composition. In some embodiments, the reference pharmaceutical composition comprises the same components as the pharmaceutical composition with the exception of one or more components that affect or increase viscosity of a composition or solution.

In certain embodiments, the pharmaceutical composition comprises sucrose at a concentration of 3% (weight/volume, 30 g/L) to 18% (weight/volume, 180 g/L). In certain embodiments, the pharmaceutical composition comprises sucrose at a concentration of 4% (weight/volume, 40 g/L).

In certain embodiments, the pharmaceutical composition comprises poloxamer 188 at a concentration of 0.001% (weight/volume, 0.01 g/L). In certain embodiments, the pharmaceutical composition comprises poloxamer 188 at a concentration of 0.0005% (weight/volume, 0.005 g/L) to 0.05% (weight/volume, 0.5 g/L). In certain embodiments, the pharmaceutical composition comprises poloxamer 188 at a concentration of 0.001% (weight/volume, 0.01 g/L). In certain embodiments, the pharmaceutical composition comprises polysorbate 20 at a concentration of 0.0005% (weight/volume, 0.05 g/L) to 0.05% (weight/volume, 0.5 g/L). In certain embodiments, the pharmaceutical composition comprises polysorbate 80 at a concentration of 0.0005% (weight/volume, 0.05 g/L) to 0.05% (weight/volume, 0.5 g/L).

In certain embodiments, the pH of the pharmaceutical composition is about 7.4. In certain embodiments, the pH of the pharmaceutical composition is about 6.0 to 9.0. In certain embodiments, the pH of the pharmaceutical composition is 7.4. In certain embodiments, the pH of the pharmaceutical composition is 6.0 to 9.0.

In certain embodiments, the pharmaceutical composition is in a hydrophobically-coated glass vial. In certain embodiments, the pharmaceutical composition is in a Cyclo Olefin Polymer (COP) vial. In certain embodiments, the pharmaceutical composition is in a Daikyo Crystal Zenith® (CZ) vial. In certain embodiments, the pharmaceutical composition is in a TopLyo coated vial.

In certain embodiments, disclosed herein is a pharmaceutical composition comprising a recombinant AAV and at least one of: (a) potassium chloride at a concentration of 0.2 g/L, (b) potassium phosphate monobasic at a concentration of 0.2 g/L, (c) sodium chloride at a concentration of 5.84 g/L, (d) sodium phosphate dibasic anhydrous at a concentration of 1.15 g/L, (e) sucrose at a concentration of 4% weight/volume (40 g/L), (f) poloxamer 188 at a concentration of 0.001% weight/volume (0.01 g/L), and (g) water, and wherein the recombinant AAV is AAV8. In some embodiments, the pharmaceutical composition does not comprise sucrose.

In some embodiments, the pharmaceutical composition comprises (a) the Construct II encoding an anti-human vascular endothelial growth factor (hVEGF) antibody and at least one of: (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water, and wherein the anti-hVEGF antibody comprises a heavy chain comprising the amino acid sequence of SEQ ID NO:2 or SEQ ID NO:4, and a light chain comprising the amino acid sequence of SEQ ID NO:1, or SEQ ID NO:3. In some embodiments, the pharmaceutical composition does not comprise sucrose.

In some embodiments, the pharmaceutical composition comprises (a) an AAV8 or AAV9 that encodes Tripeptidyl-Peptidase 1 and at least one of: (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water. In some embodiments, the pharmaceutical composition does not comprise sucrose. In some embodiments, the viscosity of the pharmaceutical composition impacts Batten-CLN2-associated vision loss.

In some embodiments, the pharmaceutical composition has desired viscosity, density, and/or osmolality that is suitable for suprachoroidal injection (for example, via a suprachoroidal drug delivery device such as a microinjector with a microneedle). In some embodiments, the pharmaceutical composition is a liquid composition. In some embodiments, the pharmaceutical composition is a frozen composition. In some embodiments, the pharmaceutical composition is a lyophilized composition from a liquid composition disclosed herein. In some embodiments, the pharmaceutical composition is a reconstituted lyophilized formulation.

In some embodiments, the pharmaceutical composition is a lyophilized composition comprising a residual moisture content between about 1% and about 7%. In some embodiments, the pharmaceutical composition is a lyophilized composition comprising a residual moisture content between about 2% and about 6%. In some embodiments, the pharmaceutical composition is a lyophilized composition comprising a residual moisture content between about 3% and about 4%. In some embodiments, the pharmaceutical composition is a lyophilized composition comprising a residual moisture content about 5%.

In certain embodiments, the pharmaceutical composition has a osmolality range of 200 mOsm/L to 660 mOsm/L. In certain embodiments, the pharmaceutical composition has a osmolality of about, of at least about, or of at most about: 200 mOsm/L, 250 mOsm/L, 300 mOsm/L, 350 mOsm/L, 400 mOsm/L, 450 mOsm/L, 500 mOsm/L, 550 mOsm/L, 600 mOsm/L, 650 mOsm/L, or 660 mOsm/L.

In certain embodiments, gene therapy constructs are supplied as a frozen sterile, single use solution of the AAV vector active ingredient in a formulation buffer. In a specific embodiment, the pharmaceutical compositions suitable for subretinal administration comprise a suspension of the recombinant (e.g., rHuGlyFabVEGFi) vector in a formulation buffer comprising a physiologically compatible aqueous buffer, a surfactant and optional excipients. In some embodiments, the construct is formulated in Dulbecco's phosphate buffered saline and poloxamer 188, pH=7.4.

4.2 Functional Properties

4.2.1 Clearance Time

The disclosure provides a pharmaceutical composition (e.g., liquid formulation comprising an AAV comprising an expression cassette encoding a transgene) resulting in a delayed clearance time from the SCS. In some embodiments, a viscous (or more viscous) pharmaceutical composition results in delayed clearance time from the SCS as compared to a non-viscous or low viscosity pharmaceutical composition. In some embodiments, a viscous (or more viscous) pharmaceutical composition results in delayed clearance time from the eye as compared to a non-viscous or low viscosity pharmaceutical composition. In some embodiments, a more viscous pharmaceutical composition results in delayed clearance time from the eye as compared to a less viscous pharmaceutical composition. In some embodiments, a more viscous pharmaceutical composition has a viscosity value that is higher than the viscosity of water. In some embodiments, a more viscous pharmaceutical composition has a viscosity value that is higher than the viscosity of a solution normally used for subretinal injection. In some embodiments, the clearance time of the pharmaceutical composition after the pharmaceutical composition is administered to the SCS is equal to or higher than the clearance time of a reference pharmaceutical composition after the reference pharmaceutical composition is administered subretinally or intravitreously. In some embodiments, the clearance time of the pharmaceutical composition after the pharmaceutical composition is administered to the SCS is equal to or higher than the clearance time of a reference pharmaceutical composition after the reference pharmaceutical composition is administered to the SCS.

In some embodiments, a pharmaceutical composition (e.g., liquid formulation comprising an AAV comprising an expression cassette encoding a transgene) results in a clearance time from the SCS of about 30 minutes to about 20 hours, about 2 hours to about 20 hours, about 30 minutes to about 24 hours, about 1 hour to about 2 hours, about 30 minutes to about 90 days, about 30 minutes to about 60 days, about 30 minutes to about 30 days, about 30 minutes to about 21 days, about 30 minutes to about 14 days, about 30 minutes to about 7 days, about 30 minutes to about 3 days, about 30 minutes to about 2 days, about 30 minutes to about 1 day, about 4 hours to about 90 days, about 4 hours to about 60 days, about 4 hours to about 30 days, about 4 hours to about 21 days, about 4 hours to about 14 days, about 4 hours to about 7 days, about 4 hours to about 3 days, about 4 hours to about 2 days, about 4 hours to about 1 day, about 4 hours to about 8 hours, about 4 hours to about 16 hours, about 4 hours to about 20 hours, about 1 day to about 90 days, about 1 day to about 60 days, about 1 day to about 30 days, about 1 day to about 21 days, about 1 day to about 14 days, about 1 day to about 7 days, about 1 day to about 3 days, about 2 days to about 90 days, about 3 days to about 90 days, about 3 days to about days, about 3 days to about 30 days, about 3 days to about 21 days, about 3 days to about 14 days, or about 3 days to about 7 days. In some embodiments, the clearance time from the SCS is of about 3 days to about 365 days, about 3 days to about 300 days, about 3 days to about 200 days, about 3 days to about 150 days, about 3 days to about 125 days, about 7 days to about 365 days, about 7 days to about 300 days, about 7 days to about 200 days, about 7 days to about 150 days, about 7 days to about 125 days. The “clearance time from the SCS” is the time required for substantially all of the pharmaceutical composition, the pharmaceutical agent, or the AAV to escape the SCS. In some embodiments, the “clearance time from the SCS” is the time required for the pharmaceutical composition, the pharmaceutical agent, or the AAV to not be detected in the SCS by any standard method (such as those described in Section 4.6 and Section 5). In some embodiments, the “clearance time from the SCS” is when the pharmaceutical composition, the pharmaceutical agent, or the AAV is present in the SCS in an amount that is at most about 2% or at most about 5% as detected by any standard method (such as those described in Section 4.6 and Section 5).

In some embodiments, the pharmaceutical composition (e.g., liquid formulation comprising an AAV comprising an expression cassette encoding a transgene) results in a clearance time from the eye of about 30 minutes to about 20 hours, about 2 hours to about 20 hours, about 30 minutes to about 24 hours, about 1 hour to about 2 hours, about 30 minutes to about 90 days, about 30 minutes to about 60 days, about 30 minutes to about 30 days, about 30 minutes to about 21 days, about 30 minutes to about 14 days, about 30 minutes to about 7 days, about 30 minutes to about 3 days, about 30 minutes to about 2 days, about 30 minutes to about 1 day, about 4 hours to about 90 days, about 4 hours to about 60 days, about 4 hours to about 30 days, about 4 hours to about 21 days, about 4 hours to about 14 days, about 4 hours to about 7 days, about 4 hours to about 3 days, about 4 hours to about 2 days, about 4 hours to about 1 day, about 4 hours to about 8 hours, about 4 hours to about 16 hours, about 4 hours to about 20 hours, about 1 day to about 90 days, about 1 day to about 60 days, about 1 day to about 30 days, about 1 day to about 21 days, about 1 day to about 14 days, about 1 day to about 7 days, about 1 day to about 3 days, about 2 days to about 90 days, about 3 days to about 90 days, about 3 days to about days, about 3 days to about 30 days, about 3 days to about 21 days, about 3 days to about 14 days, or about 3 days to about 7 days. In some embodiments, the clearance time from the eye is of about 3 days to about 365 days, about 3 days to about 300 days, about 3 days to about 200 days, about 3 days to about 150 days, about 3 days to about 125 days, about 7 days to about 365 days, about 7 days to about 300 days, about 7 days to about 200 days, about 7 days to about 150 days, about 7 days to about 125 days. The “clearance time from the eye” is the time required for substantially all of the pharmaceutical composition, the pharmaceutical agent, or the AAV to escape the eye. In some embodiments, the “clearance time from the eye” is the time required for the pharmaceutical composition, the pharmaceutical agent, or the AAV to not be detected in the eye by any method (such as those described in Section 4.6 and Section 5). In some embodiments, the “clearance time from the eye” is when the pharmaceutical composition, the pharmaceutical agent, or the AAV is present in the eye in an amount that is at most about 2% or at most about 5% as detected by any standard method (such as those described in Section 4.6 and Section 5).

In some embodiments, the clearance time is not prior to (e.g., the clearance time from the SCS or the eye does not occur before) about 30 minutes, 1 hour, 2 hours, 3 hours, 4 hours, hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days after administration of the pharmaceutical composition (e.g., a liquid formulation). In some embodiments, the clearance time is about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, days, 95 days, 100 days, 120 days, 140 days, 160 days, 180 days, 200 days, 220 days, 240 days, 260 days, 280 days, 300 days, 320 days, 340 days, 360 days, 380 days, or 400 days after administration of the pharmaceutical composition (e.g., a liquid formulation).

In some embodiments, a more viscous pharmaceutical composition (e.g., liquid formulation comprising an AAV comprising an expression cassette encoding a transgene) results in a clearance time that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when a less viscous pharmaceutical composition (reference pharmaceutical composition) is used to administer the AAV comprising the expression cassette encoding the transgene (e.g., via a subretinal administration, intravitreous administration, or to the SCS).

In some embodiments, a suprachoroidal administration of a more viscous pharmaceutical composition (e.g., liquid formulation comprising an AAV comprising an expression cassette encoding a transgene) results in a clearance time that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when a less viscous pharmaceutical composition (reference pharmaceutical composition) is used, for example, to administer the AAV comprising the expression cassette encoding the transgene by suprachoroidal administration.

In some embodiments, a suprachoroidal administration of a more viscous pharmaceutical composition (e.g., liquid formulation comprising an AAV comprising an expression cassette encoding a transgene) results in a clearance time that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when a less viscous pharmaceutical composition is used, for example, to administer the AAV comprising the expression cassette encoding the transgene by subretinal administration or by intravitreous administration.

In some embodiments, a suprachoroidal administration of a viscous (e.g., relatively viscous, medium to super high viscosity, or more viscous than water, or more viscous than a control solution, or more viscous than a solution commonly used for subretinal administration) pharmaceutical composition (e.g., liquid formulation comprising an AAV comprising an expression cassette encoding a transgene) results in a clearance time that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when the same pharmaceutical composition is used, for example, to administer the AAV comprising the expression cassette encoding the transgene via subretinal administration or via intravitreous administration.

In some embodiments, the clearance time of a relatively viscous pharmaceutical composition (e.g., a pharmaceutical composition comprising an AAV comprising an expression cassette encoding a transgene) administered by suprachoroidal injection is greater than the clearance time of the same pharmaceutical composition administered via subretinal administration or via intravitreous administration. In some embodiments, the clearance time of a more viscous pharmaceutical composition (e.g., a pharmaceutical composition comprising an AAV comprising an expression cassette encoding a transgene) administered by suprachoroidal injection is greater than a comparable less viscous pharmaceutical composition administered by suprachoroidal injection. In some embodiments, the clearance time of a more viscous pharmaceutical composition (e.g., a pharmaceutical composition comprising an AAV comprising an expression cassette encoding a transgene) administered by suprachoroidal injection is greater than a comparable less viscous pharmaceutical composition administered via subretinal administration or via intravitreous administration. In some embodiments, the clearance time of a viscous pharmaceutical composition (e.g., a pharmaceutical composition comprising an AAV comprising an expression cassette encoding a transgene) administered by suprachoroidal injection is greater than a comparable viscous pharmaceutical composition administered via subretinal administration or via intravitreous administration.

In some embodiments, the clearance time of a viscous pharmaceutical composition (e.g., a pharmaceutical composition comprising an AAV comprising an expression cassette encoding a transgene) administered by suprachoroidal injection is greater than the same pharmaceutical composition administered via subretinal administration or via intravitreous administration by at least 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

In some embodiments, the clearance time of a more viscous pharmaceutical composition (e.g., a pharmaceutical composition comprising an AAV comprising an expression cassette encoding a transgene) administered by suprachoroidal injection is greater than a comparable less viscous pharmaceutical composition (a reference pharmaceutical composition) administered by suprachoroidal injection by at least 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

In some embodiments, the clearance time of a more viscous pharmaceutical composition (e.g., a pharmaceutical composition comprising an AAV comprising an expression cassette encoding a transgene) administered by suprachoroidal injection is greater than a comparable less viscous pharmaceutical composition administered via subretinal administration or via intravitreous administration by at least 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

In some embodiments, the clearance time of the pharmaceutical composition administered via intravitreous injection or via subretinal injection is of at most about 30 minutes, 1 hours, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or at most about 400 days after administration.

In some embodiments, the clearance time of a reference pharmaceutical composition administered by intravitreous injection, subretinal injection, or to the SCS is of at most about 30 minutes, 1 hours, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, days, 100 days, 120 days, 140 days, 160 days, 180 days, 200 days, 220 days, 240 days, 260 days, 280 days, 300 days, 320 days, 340 days, 360 days, 380 days, or at most about 400 days after administration.

In some embodiments, the clearance time is the clearance time from the eye. In some embodiments, the clearance time is the clearance time from the SCS. In some embodiments, the clearance time is the clearance time from the site of injection.

4.2.2 Circumferential Spread

In some embodiments, a pharmaceutical composition (e.g., liquid formulation) localizes at the site of injection. In some embodiments, a pharmaceutical composition (e.g., liquid formulation) localizes at the site of injection for a longer period of time than a comparable less viscous pharmaceutical composition. In some embodiments, a pharmaceutical composition (e.g., liquid formulation) localizes at the site of injection for a longer period of time when injected in the SCS as compared to when the pharmaceutical composition is administered by subretinal injection or intravitreous injection. The pharmaceutical composition can have different viscosity values. In some embodiments, a viscous (or more viscous) pharmaceutical composition remains localized in the SCS for a longer period of time as compared to a non-viscous or low viscosity pharmaceutical composition.

In some embodiments, localization can be determined by evaluating circumferential spread (e.g., 2D circumferential spread). In some embodiments, a more viscous pharmaceutical composition (e.g., liquid formulation comprising an AAV comprising an expression cassette encoding a transgene) results in a circumferential spread that is at least 2 times less, at least 3 times less, at least 4 times less, at least 5 times less, at least 6 times less, at least 7 times less, at least 8 times less, at least 9 times less, at least 10 times less, at least 15 times less, at least 20 times less, at least 50 times less, at least 100 times less, at least 5% less, at least 10% less, at least 15% less, at least 20% less, at least 25% less, at least 30% less, at least 35% less, at least 40%, at least 45% less, at least 50% less, at least 55% less, at least 60% less, at least 65% less, at least 70% less, at least 75% less, at least 80% less, at least 85% less, at least 90% less, at least 95% less, at least 100% less, at least 150% less, or at least 200% less, at least 250% less, or at least 300%, at least 400% less, or at least 500% less than when a less viscous pharmaceutical composition is used to administer the AAV comprising the expression cassette encoding the transgene (e.g., by suprachoroidal injection, by subretinal injection, or by intravitreous injection).

In some embodiments, a suprachoroidal administration of a more viscous pharmaceutical composition (e.g., liquid formulation comprising an AAV comprising an expression cassette encoding a transgene) results in a circumferential spread that is at least 2 times less, at least 3 times less, at least 4 times less, at least 5 times less, at least 6 times less, at least 7 times less, at least 8 times less, at least 9 times less, at least 10 times less, at least 15 times less, at least 20 times less, at least 50 times less, at least 100 times less, at least 5% less, at least 10% less, at least 15% less, at least 20% less, at least 25% less, at least 30% less, at least 35% less, at least 40%, at least 45% less, at least 50% less, at least 55% less, at least 60% less, at least 65% less, at least 70% less, at least 75% less, at least 80% less, at least 85% less, at least 90% less, at least 95% less, at least 100% less, at least 150% less, or at least 200% less, at least 250% less, or at least 300%, at least 400% less, or at least 500% less than when a less viscous pharmaceutical composition is used, for example, to administer the AAV comprising the expression cassette encoding the transgene by suprachoroidal administration, by subretinal administration, or by intravitreous administration.

In some embodiments, a suprachoroidal administration of a viscous (e.g., relatively viscous, medium to super high viscosity, or more viscous than water, or more viscous than a control solution, or more viscous than a solution commonly used for subretinal administration) pharmaceutical composition (e.g., liquid formulation comprising an AAV comprising an expression cassette encoding a transgene) results in a circumferential spread that is at least 2 times less, at least 3 times less, at least 4 times less, at least 5 times less, at least 6 times less, at least 7 times less, at least 8 times less, at least 9 times less, at least 10 times less, at least 15 times less, at least 20 times less, at least 50 times less, at least 100 times less, at least 5% less, at least 10% less, at least 15% less, at least 20% less, at least 25% less, at least 30% less, at least 35% less, at least 40%, at least 45% less, at least 50% less, at least 55% less, at least 60% less, at least 65% less, at least 70% less, at least 75% less, at least 80% less, at least 85% less, at least 90% less, at least 95% less, at least 100% less, at least 150% less, or at least 200% less, at least 250% less, or at least 300%, at least 400% less, or at least 500% less than when the same pharmaceutical composition is used, for example, to administer the AAV comprising the expression cassette encoding the transgene by subretinal administration or by intravitreous administration.

In some embodiments, the circumferential spread can be determined 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days after the pharmaceutical composition or the reference pharmaceutical composition is administered.

4.2.3 SCS Thickness

In some embodiments, localization can be determined by evaluating SCS thickness after a pharmaceutical composition (e.g., liquid formulation) is administered to a subject. In some embodiments, a pharmaceutical composition (e.g., liquid formulation) increases the thickness of the SCS after the pharmaceutical composition (e.g., liquid formulation) is injected in the SCS. In some embodiments, an SCS expands to accommodate the infusion of a low-viscosity pharmaceutical composition (e.g., liquid formulation). In some embodiments, the infusion of a greater volume of the low-viscosity pharmaceutical composition (e.g., liquid formulation) does not cause further expansion of the SCS. In some embodiments, the greater volume of the low-viscosity fluid formulation is accommodated by increasing the area of fluid spread in the SCS without further expanding the SCS. In some embodiments, the infusion into the SCS of a viscous pharmaceutical composition (e.g., liquid formulation) can expand SCS thickness beyond the SCS thickness achieved when a low-viscosity pharmaceutical composition (e.g., liquid formulation) is infused into the SCS. In some embodiments, increasing the SCS thickness with a viscous pharmaceutical composition (e.g., liquid formulation) may ease access to the SCS, thereby easing or permitting the disposal of a device in the SCS. In some embodiments, expanding the SCS thickness allows for the pharmaceutical composition (e.g., liquid formulation) and/or the AAV encoded transgene to remain at the site of injection (localized) for a longer period of time. In some embodiments, a viscous pharmaceutical composition increases the thickness at or near the site of injection for a longer period of time as compared to a non-viscous or low viscosity pharmaceutical composition. In some embodiments, a more viscous pharmaceutical composition increases the thickness at or near the site of injection for a longer period of time as compared to a less viscous pharmaceutical composition. In some embodiments, the thickness at the site of injection after the pharmaceutical composition is administered to the SCS is equal to or higher than the thickness at the site of injection of a reference pharmaceutical composition after the reference pharmaceutical composition is administered subretinally or intravitreously. In some embodiments, the thickness at the site of injection of the pharmaceutical composition after the pharmaceutical composition is administered to the SCS is equal to or higher than the thickness at the site of injection of a reference pharmaceutical composition after the reference pharmaceutical composition is administered to the SCS.

In some embodiments, a suprachoroidal administration of a viscous (e.g., relatively viscous, medium to super high viscosity, or more viscous than water, or more viscous than a control solution, or more viscous than a solution commonly used for subretinal administration) pharmaceutical composition (e.g., liquid formulation comprising an AAV comprising an expression cassette encoding a transgene) results in an increase in the SCS thickness that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when a less viscous pharmaceutical composition is used, for example, to administer the AAV comprising the expression cassette encoding the transgene by suprachoroidal administration.

In some embodiments, a suprachoroidal administration of a more viscous pharmaceutical composition (e.g., liquid formulation comprising an AAV comprising an expression cassette encoding a transgene) results in an increase in thickness at or near the site of injection that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when a less viscous pharmaceutical composition is used, for example, to administer the AAV comprising the expression cassette encoding the transgene by subretinal administration or by intravitreous administration.

In some embodiments, a suprachoroidal administration of a viscous (e.g., relatively viscous, medium to super high viscosity, or more viscous than water, or more viscous than a control solution, or more viscous than a solution commonly used for subretinal administration) pharmaceutical composition (e.g., liquid formulation comprising an AAV comprising an expression cassette encoding a transgene) results in an increase in thickness at or near the site of injection that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when the same pharmaceutical composition is used, for example, to administer the AAV comprising the expression cassette encoding the transgene by subretinal administration or by intravitreous administration.

In some embodiments, the thickness obtained at the site of injection after a more viscous pharmaceutical composition (e.g., a pharmaceutical composition comprising an AAV comprising an expression cassette encoding a transgene) is administered by suprachoroidal injection is greater than after a comparable less viscous pharmaceutical composition is administered by suprachoroidal injection. In some embodiments, the thickness obtained at the site of injection after a more viscous pharmaceutical composition (e.g., a pharmaceutical composition comprising an AAV comprising an expression cassette encoding a transgene) is administered by suprachoroidal injection is greater than after a comparable less viscous pharmaceutical composition administered by subretinal injection or by intravitreous injection. In some embodiments, the thickness obtained at the site of injection after a viscous pharmaceutical composition (e.g., a pharmaceutical composition comprising an AAV comprising an expression cassette encoding a transgene) is administered by suprachoroidal injection is greater than after the same pharmaceutical composition administered by subretinal administration or by intravitreous administration.

In some embodiments, the thickness at or near the site of injection (e.g., thickness at or near the SCS) can be determined 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days after the pharmaceutical composition or the reference pharmaceutical composition is administered.

4.2.4 Vasodilation and Vascular Leakage

In some embodiments, a level of VEGF-induced vasodilation and/or vascular leakage after the pharmaceutical composition is administered to the SCS is equal to or less than a level of VEGF-induced vasodilation and/or vascular leakage after a reference pharmaceutical composition is administered subretinally or intravitreously. In some embodiments, a level of VEGF-induced vasodilation and/or vascular leakage after the pharmaceutical composition is administered to the SCS is equal to or lower than a level of VEGF-induced vasodilation and/or vascular leakage after the reference pharmaceutical composition is administered to the SCS. In some embodiments, a pharmaceutical composition (e.g., liquid formulation of an AAV comprising an expression cassette encoding a transgene) results in a decreased level of VEGF-induced vasodilation and/or vascular leakage after the same pharmaceutical composition is administered to the SCS as compared to after the pharmaceutical composition is administered via a subretinal administration or via an intravitreous administration. In some embodiments, a pharmaceutical composition (e.g., liquid formulation) results in a decreased level of VEGF-induced vasodilation and/or vascular leakage after the pharmaceutical composition is administered to the SCS as compared to after a comparable (less viscous) pharmaceutical composition is administered via a subretinal administration, via an intravitreous administration, or to the SCS. In some embodiments, the VEGF-induced vasodilation and/or vascular leakage is decreased by at least about 2 times, at least 3 times, at least 4 times, at least 5 times, at least 6 times, at least 7 times, at least 8 times, at least 9 times, at least 10 times, at least 15 times, at least times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%. In some embodiments, the transgene is an anti-human vascular endothelial growth factor (anti-VEGF) antibody.

In some embodiments, the VEGF-induced vasodilation and/or vascular leakage is determined about 30 minutes, 1 hour, 2 hours, 4 hours, 6 hours, 8 hours, 10 hours, 12 hours, 14 hours, 15 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or at most about 400 days after administration.

4.2.5 Rate of Transduction (or Rate of Infection) at Site of Injection

In some embodiments, the rate of transduction (or rate of infection) at the site of injection after a pharmaceutical composition is administered in the SCS is equal to or higher as compared to the rate of transduction (or rate of infection) at a site of injection after the same pharmaceutical composition is administered via a subretinal administration or via an intravenous administration. In some embodiments, the rate of transduction (or rate of infection) at the site of injection after a pharmaceutical composition is administered in the SCS is equal to or higher as compared to the rate of transduction (or rate of infection) at the site of injection after a comparable (e.g., less viscous) pharmaceutical composition (reference pharmaceutical composition) is administered via a subretinal, or intravenous administration, or to the SCS. In some embodiments, the pharmaceutical composition has a higher viscosity than the reference pharmaceutical composition (a comparable less viscous pharmaceutical composition). In some embodiments, the pharmaceutical composition and the reference pharmaceutical composition have the same vector genome concentration. In some embodiments, the pharmaceutical composition and the reference pharmaceutical composition have the same amount of genome copies. In some embodiments, the increase in the rate of transduction (or rate of infection) at the site of injection is an increase of at least about 2 times, at least 3 times, at least 4 times, at least 5 times, at least 6 times, at least 7 times, at least 8 times, at least 9 times, at least 10 times, at least times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%

In some embodiments, a level of AAV at the site of injection is equal to or higher after the pharmaceutical composition is administered suprachoroidally as compared to a level of AAV at the site of injection after the same pharmaceutical composition is administered via a subretinal administration or via an intravenous administration. In some embodiments, a level of AAV at the site of injection after the pharmaceutical composition is administered suprachoroidally is equal to or higher as compared to a level of AAV at the site of injection after a comparable (e.g., less viscous) pharmaceutical composition is administered via a subretinal, or intravenous administration, or to the SCS. In some embodiments, the pharmaceutical composition has a higher viscosity than the reference pharmaceutical composition. In some embodiments, the pharmaceutical composition and the reference pharmaceutical composition (comparable less viscous pharmaceutical composition) have the same vector genome concentration. In some embodiments, the pharmaceutical composition and a reference pharmaceutical composition have the same amount of genome copies. In some embodiments, the increase in the level of AAV at the site of injection is an increase of at least about 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

In some embodiments, the AAV level or the rate of transduction (or rate of infection) is determined about 30 minutes, 1 hour, 2 hours, 4 hours, 6 hours, 8 hours, 10 hours, 12 hours, 14 hours, 15 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or at most about 400 days after administration.

4.2.6 Transgene Expression

In some embodiments, the concentration of a transgene product is at least equal to or higher after a pharmaceutical composition is injected in the SCS as compared to after a reference (less viscous) pharmaceutical composition is injected in the SCS. In some embodiments, the concentration of a transgene product is at least equal to or higher after a pharmaceutical composition is injected in the SCS as compared to after a reference (less viscous) pharmaceutical composition is injected by subretinal injection or by intravitreous injection. In some embodiments, the concentration of a transgene product is at least equal to or higher after a pharmaceutical composition is injected in the SCS as compared to after the same pharmaceutical composition is injected by subretinal injection or by intravitreous injection.

In some embodiments, a transgene product (e.g., concentration of the transgene product) is detected in an eye (e.g., vitreous humor) for a longer period of time after a pharmaceutical composition is injected in the SCS as compared to after a comparable (less viscous) pharmaceutical composition is injected in the SCS. In some embodiments, a transgene product (e.g., concentration of the transgene product) is detected in an eye (e.g., vitreous humor) for a longer period of time after a pharmaceutical composition is injected in the SCS as compared to after a reference (less viscous) pharmaceutical composition is injected by subretinal injection or by intravitreous administration. In some embodiments, a transgene product (e.g., concentration of the transgene product) is detected in an eye (e.g., vitreous humor) for a longer period of time after a pharmaceutical composition is injected in the SCS as compared to after the same (or similar viscosity) pharmaceutical composition is injected by subretinal injection or by intravitreous injection.

In some embodiments, the longer period of time is at least 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days longer. In some embodiments, the longer period of time is about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days longer.

In some embodiments, the transgene is detected in an eye (e.g., vitreous humor) for period of time, after the pharmaceutical composition is administered in the SCS, that is at least about or about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days after the administration.

In some embodiments, the transgene is detected in an eye (e.g., vitreous humor) for a period of time (e.g., after the reference pharmaceutical composition is administered via subretinal administration or via intravitreous administration or to the SCS; or after the pharmaceutical composition is administered via subretinal or via intravitreous administration) that is at most about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, or 100 days after administration.

In some embodiments, the concentration of a transgene product in an eye (e.g., vitreous humor) can be determined about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days after the pharmaceutical composition or the reference pharmaceutical composition is administered.

In some embodiments, a suprachoroidal administration of a viscous (e.g., relatively viscous, medium to super high viscosity, or more viscous than water, or more viscous than a control solution, or more viscous than a solution commonly used for subretinal administration) pharmaceutical composition (e.g., liquid formulation comprising an AAV comprising an expression cassette encoding a transgene) results in a higher concentration of the transgene that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than after a comparable less viscous pharmaceutical composition (reference pharmaceutical composition) is used, for example, to administer the AAV comprising the expression cassette encoding the transgene by suprachoroidal administration.

In some embodiments, a suprachoroidal administration of a more viscous pharmaceutical composition (e.g., liquid formulation comprising an AAV comprising an expression cassette encoding a transgene) results in a higher concentration of the transgene that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when a comparable less viscous pharmaceutical composition (a reference pharmaceutical composition) is used, for example, to administer the AAV comprising the expression cassette encoding the transgene by subretinal administration or by intravitreous administration.

In some embodiments, a suprachoroidal administration of a viscous (e.g., relatively viscous, medium to super high viscosity, or more viscous than water, or more viscous than a control solution, or more viscous than a solution commonly used for subretinal administration) pharmaceutical composition (e.g., liquid formulation comprising an AAV comprising an expression cassette encoding a transgene) results in a higher concentration of the transgene that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when the same pharmaceutical composition is administered via subretinal administration or via intravitreous administration.

In some embodiments, the concentration of the transgene after a more viscous pharmaceutical composition (e.g., a pharmaceutical composition comprising an AAV comprising an expression cassette encoding a transgene) is administered by suprachoroidal injection is greater than after a comparable less viscous pharmaceutical composition is administered by suprachoroidal injection. In some embodiments, the concentration of the transgene after a more viscous pharmaceutical composition (e.g., a pharmaceutical composition comprising an AAV comprising an expression cassette encoding a transgene) is administered by suprachoroidal injection is greater than after a comparable less viscous pharmaceutical composition is administered by subretinal administration or via intravitreous administration. In some embodiments, the concentration of the transgene after a viscous pharmaceutical composition (e.g., a pharmaceutical composition comprising an AAV comprising an expression cassette encoding a transgene) is administered by suprachoroidal injection is greater than after the same pharmaceutical composition is administered by subretinal administration or via intravitreous administration.

4.2.7 Other Functional Properties

In some embodiments, the pharmaceutical composition described herein has a desired viscosity that is suitable for suprachoroidal injection. In some embodiments, the recombinant AAV in the pharmaceutical composition is at least as stable as the recombinant AAV in a reference pharmaceutical composition (or a comparable pharmaceutical composition). In some embodiments, the recombinant AAV in the pharmaceutical composition is at least 50% as stable as the recombinant AAV in a reference pharmaceutical composition (or a comparable pharmaceutical composition). In some embodiments, the recombinant AAV in the pharmaceutical composition has at least the same or a comparable aggregation level as the recombinant AAV in a reference pharmaceutical composition. In some embodiments, the recombinant AAV in the pharmaceutical composition has at least the same or a comparable infectivity level as the recombinant AAV in a reference pharmaceutical composition. In some embodiments, the recombinant AAV in the pharmaceutical composition has at least the same or a comparable free DNA level as the recombinant AAV in a reference pharmaceutical composition. In some embodiments, the recombinant AAV in the pharmaceutical composition has at least the same or a comparable in vitro relative potency (IVRP) as the recombinant AAV in a reference pharmaceutical composition. In some embodiments, the recombinant AAV in the pharmaceutical composition has at least the same or a comparable change in size level as the recombinant AAV in a reference pharmaceutical composition.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times more stable to freeze/thaw cycles than the same recombinant AAV in a reference pharmaceutical composition. In some embodiments, the recombinant AAV in the pharmaceutical composition is at least about 50%, 55%, 60%, 65%, 70%, 75%, 80%, 85%, 90%, 95%, or 100% as stable to freeze/thaw cycles as the same recombinant AAV in a reference pharmaceutical composition. In certain embodiments, the stability of the recombinant AAV is determined by an assay or assays disclosed in Section 4.6 and Section 5.

In certain embodiments, the recombinant AAV in the pharmaceutical composition exhibits at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times more infectivity than the same recombinant AAV in a reference pharmaceutical composition. In some embodiments, the recombinant AAV in the pharmaceutical composition has at least about 50%, 55%, 60%, 65%, 70%, 75%, 80%, 85%, 90%, 95%, or 100% the infectivity of the same recombinant AAV in a reference pharmaceutical composition. In certain embodiments, the virus infectivity of the recombinant AAV is determined by an assay or assays disclosed in the present disclosure. In certain embodiments, the size of the recombinant AAV is determined by an assay or assays disclosed in Section 4.6 and Section 5. In certain embodiments, the size is measured prior to or after freeze/thaw cycles.

In certain embodiments, the recombinant AAV in the pharmaceutical composition exhibits at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times less aggregation than the same recombinant AAV in a reference pharmaceutical composition. In some embodiments, the recombinant AAV in the pharmaceutical composition is at least about 50%, 55%, 60%, 65%, 70%, 75%, 80%, 85%, 90%, 95%, or 100% as stable over a period of time as the same recombinant AAV in a reference pharmaceutical composition. In certain embodiments, the aggregation of the recombinant AAV is determined by an assay or assays disclosed in the present disclosure. In certain embodiments, the aggregation is measured prior to or after freeze/thaw cycles. In certain embodiments, the aggregation of the recombinant AAV is determined by an assay or assays disclosed in Section 4.6.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times more stable over a period of time (e.g., when stored at −20° C. or at 37° C.), for example, at least about or about 1 week, about 2 weeks, about 3 weeks, about 4 weeks, about 1 month, about 2 months, about 3 months, about 4 months, about 5 months, about 6 months, about 7 months, about 8 months, about 9 months, about months, about 11 months, 12 months, about 15 months, about 18 months, about 24 months, about 2 years, about 3 years, about 4 years than the same recombinant AAV in a reference pharmaceutical composition. In some embodiments, the recombinant AAV in the pharmaceutical composition is at least about 50%, 55%, 60%, 65%, 70%, 75%, 80%, 85%, 90%, 95%, or 100% as stable over a period of time as the same recombinant AAV in a reference pharmaceutical composition. In certain embodiments, the stability over a period of time of the recombinant AAV is determined by an assay or assays disclosed in the present disclosure. In certain embodiments, the stability over a period of time of the recombinant AAV is determined by an assay or assays disclosed in Section 4.6 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 higher in in vitro relative potency (IVRP) than the same recombinant AAV in a reference pharmaceutical composition (e.g., when stored at −20° C. or at 37° C.). In some embodiments, the recombinant AAV in the pharmaceutical composition has about the same in vitro relative potency (IVRP) as the same recombinant AAV in a reference pharmaceutical composition. In some embodiments, the recombinant AAV in the pharmaceutical composition has about 50%, 55%, 60%, 65%, 70%, 75%, 80%, 85%, 90%, 95%, or 100% in vitro relative potency (IVRP) as the same recombinant AAV in a reference pharmaceutical composition. In certain embodiments, the in vitro relative potency (IVRP) of the recombinant AAV is determined by an assay or assays disclosed in the present disclosure. In certain embodiments, the in vitro relative potency (IVRP) is measured prior to or after freeze/thaw cycles. In certain embodiments, the in vitro relative potency (IVRP) of the recombinant AAV is determined by an assay or assays disclosed in Section 4.6.

In certain embodiments, the recombinant AAV in the pharmaceutical composition has at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times less free DNA than the same recombinant AAV in a reference pharmaceutical composition. In some embodiments, the recombinant AAV in the pharmaceutical composition has about the same amount of free DNA as the same recombinant AAV in a reference pharmaceutical composition. In some embodiments, the recombinant AAV in the pharmaceutical composition has about not more than two times the amount of free DNA as the same recombinant AAV in a reference pharmaceutical composition. In some embodiments, the recombinant AAV in the pharmaceutical composition has about 50%, 55%, 60%, 65%, 70%, 75%, 80%, 85%, 90%, 95%, or 100% the amount of free DNA as the same recombinant AAV in a reference pharmaceutical composition. In some embodiments, the recombinant AAV in the pharmaceutical composition has at least about 50% more, about 25% more, about 15% more, about 10% more, about 5% more, about 4% more, about 3% more, about 2% more, about 1% more, about 0% more, about 1% less, about 2% less, about 5% less, about 7% less, about 10% less, about 2 times more, about 3 times more, about 2 times less, or about 3 times less free DNA than the same recombinant AAV in a reference pharmaceutical composition. In certain embodiments, the free DNA of the recombinant AAV is determined by an assay or assays disclosed in Section 4.6 and Section 5.

In certain embodiments, the recombinant AAV in the pharmaceutical composition has at most 20%, 15%, 10%, 8%, 5%, 4%, 3%, 2%, or 1% change in size over a period of time (e.g., when stored at −20° C. or at 37° C.), for example, at least about or about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years. In certain embodiments, the size of the recombinant AAV is determined by an assay or assays disclosed in the present disclosure. In certain embodiments, the size is measured prior to or after freeze/thaw cycles. In certain embodiments, the size of the recombinant AAV is determined by an assay or assays disclosed in Section 4.6.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times more stable than the same recombinant AAV in a reference pharmaceutical composition (e.g., when stored at −20° C. or at 37° C.). In some embodiments, the recombinant AAV in the pharmaceutical composition is about as stable as the same recombinant AAV in a reference pharmaceutical composition (e.g., when stored at −20° C. or at 37° C.). In some embodiments, the recombinant AAV in the pharmaceutical composition is at least about 50%, 55%, 60%, 65%, 70%, 75%, 80%, 85%, 90%, 95%, or 99% as stable as the same recombinant AAV in a reference pharmaceutical composition (e.g., when stored at −20° C. or at 37° C.). In certain embodiments, the stability of the recombinant AAV is determined by an assay or assays disclosed in Section 4.6.

In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23, or 24 months without loss of stability as determined, e.g. by an assay or assays disclosed in Section 4.6 or. In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23, or 24 months at 4° C. without loss of stability. In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23, or 24 months at ≤60° C. without loss of stability. In certain embodiments, a pharmaceutical composition provided herein is capable of being stored for 1, 2, 3, 4, 5, 6, 7, 8, 9, 12, 13, 14, 15, 16, 17, 18, 19, 20, 21, 22, 23, or 24 months at −80° C. without loss of stability. In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23, or 24 months at 4° C. after having been stored at −20° C. for 1, 2, 3, 4, 5, 6, 7, 8, 9, 10, or 12 months without loss of stability.

In certain embodiments, a pharmaceutical composition provided herein is capable of being first stored for 1, 2, 3, 4, 5, 6, 7, 8, 9, 10, 12, 13, 14, 15, 16, 17, 18, 19, 20, 21, 22, 23, or 24 months at −80° C., then being thawed and, after thawing, being stored at 2-10° C., 4-8° C., 2° C., 3° C., 4° C., 5° C., 6° C., 7° C., 8° C. or 9° C. for 1, 2, 3, 4, 5, 6, 7, 8, 9, 10, or 12 additional months without loss of stability as determined, e.g., by an assay or assays disclosed in Section 4.6 or. In certain embodiments, a pharmaceutical composition provided herein is capable of being first stored for 1, 2, 3, 4, 5, 6, 7, 8, 9, 10, 12, 13, 14, 15, 16, 17, 18, 19, 20, 21, 22, 23, or 24 months at −80° C., then being thawed and, after thawing, being stored at about 4° C. for 1, 2, 3, 4, 5, 6, 7, 8, 9, 10, or 12 additional months without loss of stability as determined, e.g., by an assay or assays disclosed in Section 4.6 or 5. In certain embodiments, a pharmaceutical composition provided herein is capable of being first stored for 1, 2, 3, 4, 5, 6, 7, 8, 9, 10, 12, 13, 14, 15, 16, 17, 18, 19, 21, 22, 23, or 24 months at ≤60° C., then being thawed and, after thawing, being stored at about 4° C. for 1, 2, 3, 4, 5, 6, 7, 8, 9, 10, or 12 additional months without loss of stability as determined, e.g., by an assay or assays disclosed in Section 4.6 or 5.

Effects of the methods or pharmaceutical compositions provided herein may be monitored by measuring signs of vision loss, infection, inflammation and other safety events, including retinal detachment. In some embodiments, different pharmaceutical compositions (e.g., liquid formulation) having different viscosity (e.g., ranging from low viscosity to very high viscosity) can be used to deliver the vector in the SCS. In some embodiments, vectors delivered using a medium to high viscosity pharmaceutical compositions (e.g., liquid formulation) are more effective than vectors delivered using a low viscosity pharmaceutical composition (e.g., liquid formulation) (e.g., when administered in the SCS). In some embodiments, vectors delivered using a medium to high viscosity formulation results in improved vision as compared to vectors delivered using a low viscosity formulation.

Effects of the methods or pharmaceutical compositions provided herein may also be measured by a change from baseline in National Eye Institute Visual Functioning Questionnaire, the Rasch-scored version (NEI-VFQ-28-R) (composite score; activity limitation domain score; and socio-emotional functioning domain score). In some embodiments, effects of the methods provided herein may also be measured by a change from baseline in National Eye Institute Visual Functioning Questionnaire 25-item version (NEI-VFQ-25) (composite score and mental health subscale score). In some embodiments, effects of the methods provided herein may also be measured by a change from baseline in Macular Disease Treatment Satisfaction Questionnaire (MacTSQ) (composite score; safety, efficacy, and discomfort domain score; and information provision and convenience domain score).

In specific embodiments, the efficacy of a method or vector (vector formulation) described herein is reflected by an improvement in vision at about 4 weeks, 12 weeks, 6 months, 12 months, 24 months, 36 months, or at other desired timepoints. In a specific embodiment, the improvement in vision is characterized by an increase in BCVA, for example, an increase by 1 letter, 2 letters, 3 letters, 4 letters, 5 letters, 6 letters, 7 letters, 8 letters, 9 letters, 10 letters, 11 letters, or 12 letters, or more. In a specific embodiment, the improvement in vision is characterized by a 5%, 10%, 15%, 20%, 30%, 40%, 50% or more increase in visual acuity from baseline.

In specific embodiments, there is no inflammation in the eye after treatment or little inflammation in the eye after treatment (for example, an increase in the level of inflammation by 10%, 5%, 2%, 1% or less from baseline).

4.3 Dosage and Mode of Administration

In one aspect, provided herein is a method of suprachoroidal administration for treating a pathology of the eye, comprising administering to the suprachoroidal space in the eye of a human subject in need of treatment a recombinant viral vector comprising a nucleotide sequence encoding a therapeutic product such that the therapeutic product is expressed and results in treatment of the pathology of the eye. In certain embodiments, the administering step is by injecting the recombinant viral vector into the suprachoroidal space using a suprachoroidal drug delivery device. In certain embodiments, the suprachoroidal drug delivery device is a microinjector. In some embodiments, a pharmaceutical composition or a reference pharmaceutical composition provided herein is suitable for administration by one, two or more routes of administration (e.g., suitable for suprachoroidal and subretinal administration).

In certain embodiments, the vector genome concentration (VGC) of the pharmaceutical composition (or the reference pharmaceutical composition) is about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 2×10¹¹ GC/mL, about 2.4×10¹¹ GC/mL, about 2.5×10¹¹ GC/mL, about 3×10¹¹ GC/mL, about 3.2×10¹¹ GC/mL, about 6.2×10¹¹ GC/mL, about 6.5×10¹¹ GC/mL, about 1×10¹² GC/mL, about 2.5×10¹² GC/mL, about 3×10¹² GC/mL, about 5×10¹² GC/mL, about 1.5×10¹³ GC/mL, about 2×10¹³ GC/mL or about 3×10¹³ GC/mL.

In certain embodiments, the vector genome concentration (VGC) of the pharmaceutical composition (or the reference pharmaceutical composition) is about 3×10⁹ GC/mL, 4×10⁹ GC/mL, 5×10⁹ GC/mL, 6×10⁹ GC/mL, 7×10⁹ GC/mL, 8×10⁹ GC/mL, 9×10⁹ GC/mL, about 1×10¹⁰ GC/mL, about 2×10¹⁰ GC/mL, about 3×10¹⁰ GC/mL, about 4×10¹⁰ GC/mL, about 5×10¹⁰ GC/mL, about 6×10¹⁰ GC/mL, about 7×10¹⁰ GC/mL, about 8×10¹⁰ GC/mL, about 9×10¹⁰ GC/mL, about 1×10¹¹ GC/mL, about 2×10¹¹ GC/mL, about 3×10¹¹ GC/mL, about 4×10¹¹ GC/mL, about 5×10¹¹ GC/mL, about 6×10¹¹ GC/mL, about 7×10¹¹ GC/mL, about 8×10¹¹ GC/mL, about 9×10¹¹ GC/mL, about 1×10¹² GC/mL, about 2×10¹² GC/mL, about 3×10¹² GC/mL, about 4×10¹² GC/mL, about 5×10¹² GC/mL, about 6×10¹² GC/mL, about 7×10¹² GC/mL, about 8×10¹² GC/mL, about 9×10¹² GC/mL, about 1×10¹³ GC/mL, about 1.5×10¹³ GC/mL, about 2×10¹³ GC/mL, about 3×10¹³ GC/mL.

In some embodiments, the volume of the pharmaceutical composition (e.g., liquid formulation) is any volume capable of reducing the minimum force to separate the sclera and choroid. In some embodiments, the volume of the pharmaceutical composition (e.g., liquid formulation) is about 50 μL to about 1000 μL, 50 μL to about 500 μL, 50 μL to about 400 μL, 50 μL to about 350 μL, 50 μL to about 300 μL, about 50 μL to about 275 μL, about 50 μL to about 250 μL, about 50 μL to about 225 μL, about 50 μL to about 200 μL, about 50 μL to about 175 μL, about 50 μL to about 150 μL, about 60 μL to about 140 μL, about 70 μL to about 130 μL, about 80 μL to about 120 μL, about 90 μL to about 110 μL, or about 100 μL.

Currently available technologies for suprachoroidal space (SCS) delivery exist. Preclinically, SC injections have been achieved with scleral flap technique, catheters and standard hypodermic needles, as well as with microneedles. A hollow-bore 750 um-long microneedle (Clearside Biomedical, Inc.) can be inserted at the pars, and has shown promise in clinical trials. A microneedle designed with force-sensing technology can be utilized for SC injections, as described by Chitnis, et al. (Chitnis, G. D., et al. A resistance-sensing mechanical injector for the precise delivery of liquids to target tissue. Nat Biomed Eng 3, 621-631 (2019). https://doi.org/10.1038/s41551-019-0350-2). Oxular Limited is developing a delivery system (Oxulumis) that advances an illuminated cannula in the suprachoroidal space. The Orbit device (Gyroscope) is a specially-designed system enabling cannulation of the suprachoroidal space with a flexible cannula. A microneedle inside the cannula is advanced into the subretinal space to enable targeted dose delivery. Ab interno access to the SCS can also be achieved using micro-stents, which serve as minimally-invasive glaucoma surgery (MIGS) devices. Examples include the CyPass® Micro-Stent (Alcon, Fort Worth, Texas, US) and iStent® (Glaukos), which are surgically implanted to provide a conduit from the anterior chamber to the SCS to drain the aqueous humor without forming a filtering bleb. Other devices contemplated for suprachoroidal delivery include those described in UK Patent Publication No. GB 2531910A and U.S. Pat. No. 10,912,883 B2.

In some embodiments, the suprachoroidal drug delivery device is a syringe with a 1 millimeter 30 gauge needle. In some embodiments, the syringe has a larger circumference (e.g., 29 gauge needle). During an injection using this device, the needle pierces to the base of the sclera and fluid containing drug enters the suprachoroidal space, leading to expansion of the suprachoroidal space. As a result, there is tactile and visual feedback during the injection. Following the injection, the fluid flows posteriorly and absorbs dominantly in the choroid and retina. This results in the production of transgene protein from all retinal cell layers and choroidal cells. Using this type of device and procedure allows for a quick and easy in-office procedure with low risk of complications.

In some embodiments, a microneedle or syringe is selected based on the viscosity of a pharmaceutical composition (e.g., liquid formulation). In some embodiments, a microneedle is selected based on the pressure resulted in the eye (e.g., in the SCS) when a pharmaceutical composition (e.g., liquid formulation) is administered. For example, a pharmaceutical composition (e.g., liquid formulation) having medium or high viscosity may benefit from the use of a wider microneedle for injection. In some embodiments, the pressure in the SCS is lower when a wider microneedle is used as compared to the pressure obtained when a narrower microneedle is used. In some embodiments, 10 gauge needle, 11 gauge needle, 12 gauge needle, 13 gauge needle, 14 gauge needle, 15 gauge needle, 16 gauge needle, 17 gauge needle, 18 gauge needle, 19 gauge needle, 20 gauge needle, 21 gauge needle, 22 gauge needle, 23 gauge needle, 24 gauge needle, 25 gauge needle, 26 gauge needle, 27 gauge needle, 28 gauge needle, 29 gauge needle, 30 gauge needle, 31 gauge needle, 32 gauge needle, 33 gauge needle, or 34 gauge needle is used. In some embodiments, a 27 gauge needle is used. In some embodiments, a 28 gauge needle is used. In some embodiments, a 29 gauge needle is used. In some embodiments, a 30 gauge needle is used. In some embodiments, a 31 gauge needle is used. In some embodiments, a gauge that is smaller than a 27 gauge needle is used. In some embodiments, a gauge that is larger than a 27 gauge needle is used. In some embodiments, a gauge that is smaller than a 30 gauge needle is used. In some embodiments, a gauge that is higher than a 30 gauge needle is used.

In some embodiments, the pressure during administration of a pharmaceutical composition is about 10 PSI, 15 PSI, 20 PSI, 25 PSI, 30 PSI, 35 PSI, 40 PSI, 45 PSI, 50 PSI, 55 PSI, 60 PSI, 65 PSI, 70 PSI, 75 PSI, 80 PSI, 85 PSI, 90 PSI, 95 PSI, 100 PSI, 150 PSI, or 200 PSI. In some embodiments, the pressure during administration of a pharmaceutical composition is not greater than about 10 PSI, 15 PSI, 20 PSI, 25 PSI, 30 PSI, 35 PSI, 40 PSI, 45 PSI, 50 PSI, 55 PSI, 60 PSI, 65 PSI, 70 PSI, 75 PSI, 80 PSI, 85 PSI, 90 PSI, 95 PSI, 100 PSI, 150 PSI, or 200 PSI. In some embodiments, the pressure to open the SCS during administration of a pharmaceutical composition is not greater than about 10 PSI, 15 PSI, 20 PSI, 25 PSI, 30 PSI, 35 PSI, 40 PSI, 45 PSI, 50 PSI, 55 PSI, 60 PSI, 65 PSI, 70 PSI, 75 PSI, 80 PSI, 85 PSI, 90 PSI, 95 PSI, 100 PSI, 150 PSI, or 200 PSI. In some embodiments, the pressure during administration of a pharmaceutical composition (or the pressure required to open the SCS) is between 20 PSI and PSI, 20 PSI and 75 PSI, 20 PSI and 40 PSI, 10 PSI and 40 PSI, 10 PSI and 100 PSI, or 10 PSI and 80 PSI. In some embodiments, the pressure decreases as the rate of injection decreases (e.g., pressure decreases from a 4 seconds rate of injection to a 10 seconds rate of injection). In some embodiments, the pressure decreases as the size of the needle increases. In some embodiments, the pressure increases as the viscosity increases.

Doses that maintain a concentration of the transgene product at a Cmin of at least 0.330 μg/mL in the eye (e.g., Vitreous humor), or 0.110 μg/mL in the Aqueous humour (the anterior chamber of the eye) for three months are desired; thereafter, Vitreous Cmin concentrations of the transgene product ranging from 1.70 to 6.60 μg/mL, and/or Aqueous Cmin concentrations ranging from 0.567 to 2.20 μg/mL should be maintained. However, because the transgene product is continuously produced (under the control of a constitutive promoter or induced by hypoxic conditions when using an hypoxia-inducible promoter), maintenance of lower concentrations can be effective. Transgene concentrations can be measured directly in patient samples of fluid collected from a bodily fluid, ocular fluid, vitreous humor, or the anterior chamber, or estimated and/or monitored by measuring the patient's serum concentrations of the transgene product—the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54: 1616-1624, at p. 1621 and Table 5 at p. 1623, which is incorporated by reference herein in its entirety).

In certain embodiments, dosages are measured by genome copies per ml (GC/mL) or the number of genome copies administered to the eye of the patient (e.g., administered suprachoroidally). In some embodiments, 2.4×10¹¹ GC/mL to 1×10¹³ GC/mL are administered, 2.4×10¹¹ GC/mL to 5×10¹¹ GC/mL are administered, 5×10¹¹ GC/mL to 1×10¹² GC/mL are administered, 1×10¹² GC/mL to 5×10¹² GC/mL are administered, or 5×10¹² GC/mL to 1×10¹³ GC/mL are administered. In some embodiments, 1.5×10¹³ GC/mL to 3×10¹³ GC/mL are administered. In some embodiments, about 2.4×10¹¹ GC/mL, about 5×10¹¹ GC/mL, about 1×10¹² GC/mL, about 2.5×10¹² GC/mL, about 5×10¹² GC/mL, about 1×10¹³ GC/mL, or about 1.5×10¹³ GC/mL are administered. In some embodiments, 1×10⁹ to 1×10¹² genome copies are administered. In some embodiments, 3×10⁹ to 2.5×10¹¹ genome copies are administered. In specific embodiments, 1×10⁹ to 2.5×10¹¹ genome copies are administered. In specific embodiments, 1×10⁹ to 1×10¹¹ genome copies are administered. In specific embodiments, 1×10⁹ to 5×10⁹ genome copies are administered. In specific embodiments, 6×10⁹ to 3×10¹⁰ genome copies are administered. In specific embodiments, 4×10¹⁰ to 1×10¹¹ genome copies are administered. In specific embodiments, 2×10¹¹ to 1.5×10¹² genome copies are administered. In a specific embodiment, about 3×10⁹ genome copies are administered (which corresponds to about 1.2×10¹⁰ GC/mL in a volume of 250 μl). In another specific embodiment, about 1×10¹⁰ genome copies are administered (which corresponds to about 4×10¹⁰ GC/mL in a volume of 250 μl). In another specific embodiment, about 6×10¹⁰ genome copies are administered (which corresponds to about 2.4×10¹¹ GC/mL in a volume of 250 μl). In another specific embodiment, about 6.4×10¹⁰ genome copies are administered (which corresponds to about 3.2×10¹¹ GC/mL in a volume of 200 μl). In another specific embodiment, about 1.3×10¹¹ genome copies are administered (which corresponds to about 6.5×10¹¹ GC/mL in a volume of 200 μl). In some embodiments, about 6.4×10¹⁰ genome copies are administered per eye, or per dose, or per route of administration. In some embodiments, about 6.4×10¹⁰ genome copies is the total number of genome copies administered. In some embodiments, about 1.3×10¹¹ genome copies are administered per eye, or per dose, or per route of administration. In some embodiments, about 1.3×10¹¹ genome copies is the total number of genome copies administered. In some embodiments, about 2.5×10¹¹ genome copies are administered per eye, or per dose, or per route of administration. In some embodiments, about 2.5×10¹¹ genome copies is the total number of genome copies administered. In some embodiments, about 5×10¹¹ genome copies are administered per eye, or per dose, or per route of administration. In some embodiments, about 5×10¹¹ genome copies is the total number of genome copies administered. In some embodiments, about 3×10¹² genome copies are administered per eye, or per dose, or per route of administration. In some embodiments, about 3×10¹² genome copies is the total number of genome copies administered. In another specific embodiment, about 1.6×10¹¹ genome copies are administered (which corresponds to about 6.2×10¹¹ GC/mL in a volume of 250 μl). In another specific embodiment, about 1.55×10¹¹ genome copies are administered (which corresponds to about 6.2×10¹¹ GC/mL in a volume of 250 μl). In another specific embodiment, about 1.6×10¹¹ genome copies are administered (which corresponds to about 6.4×10¹¹ GC/mL in a volume of 250 μl). In another specific embodiment, about 2.5×10¹¹ genome copies (which corresponds to about 1.0×10¹² in a volume of 250 μl) are administered. In another specific embodiment, about 2.5×10¹¹ genome copies are administered (which corresponds to about 2.5×10¹² GC/mL in a volume of 100 μl). In another specific embodiment, about 3×10¹¹ genome copies are administered (which corresponds to about 3×10¹² GC/mL in a volume of 100 μl). In another specific embodiment, about 5×10¹¹ genome copies are administered (which corresponds to about 5×10¹² GC/mL in a volume of 200 μl). In another specific embodiment, about 6×10¹¹ genome copies are administered (which corresponds to about 3×10¹² GC/mL in a volume of 200 μl). In another specific embodiment, about 6×10¹¹ genome copies are administered (which corresponds to about 6×10¹² GC/mL in a volume of 100 μl). In another specific embodiment, about 1.5×10¹² genome copies are administered (which corresponds to about 1.5×10¹³ GC/mL in a volume of 100 μl).

In certain embodiments, about 6.0×10¹⁰ genome copies per administration, or per eye are administered. In certain embodiments, about 6.4×10¹⁰ genome copies per administration, or per eye are administered. In certain embodiments, about 1.3×10¹¹ genome copies per administration, or per eye are administered. In certain embodiments, about 1.5×10¹¹ genome copies per administration, or per eye are administered. In certain embodiments, about 1.6×10¹¹ genome copies per administration, or per eye are administered. In certain embodiments, about 2.5×10¹¹ genome copies per administration, or per eye are administered. In certain embodiments, about 3×10¹¹ genome copies per administration, or per eye are administered. In certain embodiments, about 5.0×10¹¹ genome copies per administration, or per eye are administered. In certain embodiments, about 6×10¹¹ genome copies per administration, or per eye are administered. In some embodiments, about 1.5×10¹² genome copies are administered per eye, or per dose, or per route of administration. In some embodiments, about 1.5×10¹² genome copies is the total number of genome copies administered. In certain embodiments, about 3×10¹² genome copies per administration, or per eye are administered. In certain embodiments, about 1.0×10¹² GC/mL per administration, or per eye are administered. In certain embodiments, about 2.5×10¹² GC/mL per administration, or per eye are administered. In certain embodiments, about 3×10¹² GC/mL per administration, or per eye are administered. In certain embodiments, about 3.0×10¹³ genome copies per administration, or per eye are administered. In certain embodiments, up to 3.0×10¹³ genome copies per administration, or per eye are administered.

In certain embodiments, about 1.5×10¹¹ genome copies per administration, or per eye are administered by suprachoroidal injection. In certain embodiments, about 2.5×10¹¹ genome copies per administration, or per eye are administered by suprachoroidal injection. In certain embodiments, about 3×10¹¹ genome copies per administration, or per eye are administered by suprachoroidal injection. In certain embodiments, about 5.0×10¹¹ genome copies per administration, or per eye are administered by suprachoroidal injection. In certain embodiments, about 6×10¹¹ genome copies per administration, or per eye are administered by suprachoroidal injection. In certain embodiments, about 1.5×10¹² genome copies per administration, or per eye are administered by suprachoroidal injection. In certain embodiments, about 3×10¹² genome copies per administration, or per eye are administered by suprachoroidal injection. In certain embodiments, about 2.5×10¹¹ genome copies per eye are administered by a single suprachoroidal injection. In certain embodiments, about 3×10¹¹ genome copies per administration, or per eye are administered by a single suprachoroidal injection. In certain embodiments, about 3×10¹¹ genome copies per administration, or per eye are administered by a single suprachoroidal injection in a volume of 100 μl. In certain embodiments, about 3×10¹¹ genome copies per administration, or per eye are administered by a single suprachoroidal injection in a volume of 200 μl. In certain embodiments, about 3×10¹¹ genome copies per administration, or per eye are administered by double suprachoroidal injections. In certain embodiments, about 3×10¹¹ genome copies per administration, or per eye are administered by double suprachoroidal injections, wherein each injection is in a volume of 50 μl. In certain embodiments, about 3×10¹¹ genome copies per administration, or per eye are administered by double suprachoroidal injections, wherein each injection is in a volume of 100 μl. In certain embodiments, about 5.0×10¹¹ genome copies per administration, or per eye are administered by double suprachoroidal injections. In certain embodiments, about 6×10¹¹ genome copies per administration, or per eye are administered by a single suprachoroidal injection. In certain embodiments, about 6×10¹¹ genome copies per administration, or per eye are administered by a single suprachoroidal injection in a volume of 100 μl. In certain embodiments, about 6×10¹¹ genome copies per administration, or per eye are administered by a single suprachoroidal injection in a volume of 200 μl. In certain embodiments, about 6×10¹¹ genome copies per administration, or per eye are administered by double suprachoroidal injections. In certain embodiments, about 6×10¹¹ genome copies per administration, or per eye are administered by double suprachoroidal injections, wherein each injection is in a volume of 50 μl. In certain embodiments, about 6×10¹¹ genome copies per administration, or per eye are administered by double suprachoroidal injections, wherein each injection is in a volume of 100 μl. In certain embodiments, about 3.0×10¹³ genome copies per administration, or per eye are administered by suprachoroidal injection. In certain embodiments, up to 3.0×10¹³ genome copies per administration, or per eye are administered by suprachoroidal injection. In certain embodiments, about 2.5×10¹² GC/mL per eye are administered by a single suprachoroidal injection in a volume of 100 μl. In certain embodiments, about 2.5×10¹² GC/mL per eye are administered by double suprachoroidal injections, wherein each injection is in a volume of 100 μl. In certain embodiments, about 1.5×10¹³ GC/mL per eye are administered by a single suprachoroidal injection in a volume of 100 μl.

In certain embodiments, the recombinant viral vector is administered by double suprachoroidal injections. In certain embodiments, the first injection in the right eye is administered in the superior temporal quadrant (i.e., between the 10 o'clock and 11 o'clock positions), and the second injection in the same eye is administered in the inferior nasal quadrant (i.e., between the 4 o'clock and 5 o'clock positions). In certain embodiments, the first injection in the right eye is administered in the inferior nasal quadrant (i.e., between the 4 o'clock and 5 o'clock positions), and the second injection in the same eye is administered in the superior temporal quadrant (i.e., between the 10 o'clock and 11 o'clock positions). In certain embodiments, the first injection in the left eye is administered in the superior temporal quadrant (i.e., between the 1 o'clock and 2 o'clock positions), and the second injection in the same eye is administered in the inferior nasal quadrant (i.e., between the 7 o'clock and 8 o'clock positions). In certain embodiments, the first injection in the left eye is administered in the inferior nasal quadrant (i.e., between the 7 o'clock and 8 o'clock positions), and the second injection in the same eye is administered in the superior temporal quadrant (i.e., between the 1 o'clock and 2 o'clock positions).

In certain embodiments, the recombinant viral vector is administered by a single suprachoroidal injection. In certain embodiments, the single injection in the right eye is administered in the superior temporal quadrant (i.e., between the 10 o'clock and 11 o'clock positions). In certain embodiments, the single injection in the right eye is administered in the inferior nasal quadrant (i.e., between the 4 o'clock and 5 o'clock positions). In certain embodiments, the single injection in the left eye is administered in the superior temporal quadrant (i.e., between the 1 o'clock and 2 o'clock positions). In certain embodiments, the single injection in the left eye is administered in the inferior nasal quadrant (i.e., between the 7 o'clock and 8 o'clock positions).

In some embodiments, the pharmaceutical composition or the reference pharmaceutical composition is administered to a human subject (e.g., suprachoroidally, subretinally, or intravitreously) once, twice, three times, four times, five times, six times, seven times, eight times, nine times, ten times, fifteen times, twenty times, twenty five times, or thirty times. In some embodiments, the pharmaceutical composition or the reference pharmaceutical composition is administered to a human subject once in one day, twice in one day, three times in one day, four times in one day, five times in one day, six times in one day, or seven times in one day. In some embodiments, the same amount of AAV genome copies are administered per administration. For example, the same genome copies are administered suprachoroidally, subretinally, or intravitreously. In some embodiments, the same total amount of AAV genome copies are administered. For example, the same total amount of AAV genome copies are administered suprachoroidally, subretinally, or intravitreously regardless of the number of total administrations (e.g., if subretinal administration is performed once and suprachoroidal administration is performed twice, the genome copies in the one subretinal administration is the same as the genome copies in both suprachoroidal administrations combined).

As used herein and unless otherwise specified, the term “about” means within plus or minus 10% of a given value or range

4.4 Constructs and Formulations

In some embodiments, the recombinant vectors provided herein comprise the following elements in the following order: a) a constitutive or a hypoxia-inducible promoter sequence, and b) a sequence encoding the transgene (e.g., therapeutic product). In certain embodiments, the recombinant vectors provided herein comprise the following elements in the following order: a) a first ITR sequence, b) a first linker sequence, c) a constitutive or a hypoxia-inducible promoter sequence, d) a second linker sequence, e) an intron sequence, f) a third linker sequence, g) a first UTR sequence, h) a sequence encoding the transgene (e.g., an anti-VEGF antigen-binding fragment moiety), i) a second UTR sequence, j) a fourth linker sequence, k) a poly A sequence, l) a fifth linker sequence, and m) a second ITR sequence.

In certain embodiments, the recombinant vectors provided herein comprise the following elements in the following order: a) a first ITR sequence, b) a first linker sequence, c) a constitutive or a hypoxia-inducible promoter sequence, d) a second linker sequence, e) an intron sequence, f) a third linker sequence, g) a first UTR sequence, h) a sequence encoding the transgene (e.g., an anti-VEGF antigen-binding fragment moiety), i) a second UTR sequence, j) a fourth linker sequence, k) a poly A sequence, l) a fifth linker sequence, and m) a second ITR sequence, wherein the transgene comprises the signal peptide of VEGF (SEQ ID NO: 5), and wherein the transgene encodes a light chain and a heavy chain sequence separated by a cleavable F/F2A sequence.

In some embodiments, the AAV (AAV viral vectors) provided herein comprise the following elements in the following order: a) a constitutive or a hypoxia-inducible promoter sequence, and b) a sequence encoding the transgene (e.g., an anti-VEGF antigen-binding fragment moiety). In some embodiments, the transgene is a fully human post-translationally modified (HuPTM) antibody against VEGF. In some embodiments, the fully human post-translationally modified antibody against VEGF is a fully human post-translationally modified antigen-binding fragment of a monoclonal antibody (mAb) against VEGF (“HuPTMFabVEGFi”). In some embodiments, the HuPTMFabVEGFi is a fully human glycosylated antigen-binding fragment of an anti-VEGF mAb (“HuGlyFabVEGFi”). In an alternative embodiment, full-length mAbs can be used. In some embodiments, the AAV used for delivering the transgene should have a tropism for human retinal cells or photoreceptor cells. Such AAV can include non-replicating recombinant adeno-associated virus vectors (“rAAV”), particularly those bearing an AAV8 capsid are preferred. In a specific embodiment, the viral vector or other DNA expression construct described herein is Construct I, wherein the Construct I comprises the following components: (1) AAV8 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides. In some embodiments, the viral vector comprises a signal peptide. In some embodiments, the signal peptide is MYRMQLLLLIALSLALVTNS (SEQ ID NO: 55). In some embodiments, the signal peptide is derived from IL-2 signal sequence. In some embodiments, the viral vector comprises a signal peptide from any signal peptide disclosed in Table 1, such as MNFLLSWVHW SLALLLYLHH AKWSQA (VEGF-A signal peptide) (SEQ ID NO: 5); MERAAPSRRV PLPLLLLGGL ALLAAGVDA (Fibulin-1 signal peptide) (SEQ ID NO: 6); MAPLRPLLIL ALLAWVALA (Vitronectin signal peptide) (SEQ ID NO: 7); MRLLAKIICLMLWAICVA (Complement Factor H signal peptide) (SEQ ID NO: 8); MRLLAFLSLL ALVLQETGT (Opticin signal peptide) (SEQ ID NO: 9); MKWVTFISLLFLFSSAYS (Albumin signal peptide) (SEQ ID NO: 22); MAFLWLLSCWALLGTTFG (Chymotrypsinogen signal peptide) (SEQ ID NO: 23); MYRMQLLSCIALILALVTNS (Interleukin-2 signal peptide) (SEQ ID NO: 24); MNLLLILTFVAAAVA (Trypsinogen-2 signal peptide) (SEQ ID NO: 25); or MYRMQLLLLIALSLALVTNS (mutant Interleukin-2 signal peptide) (SEQ ID NO: 55). In another specific embodiment, the viral vector or other DNA expression construct described herein is Construct II, wherein the Construct II comprise the following components: (1) AAV2 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides. In some embodiments, the anti-hVEGF antibody comprises a heavy chain comprising the amino acid sequence of SEQ ID NO:2 or SEQ ID NO:4, and a light chain comprising the amino acid sequence of SEQ ID NO:1, or SEQ ID NO:3.

In some embodiments, the viral vector or other expression construct suitable for packaging in an AAV capsid, comprises (1) AAV inverted terminal repeats (ITRs) flank the expression cassette; (2) regulatory control elements, consisting essentially of one or more enhancers and/or promoters, d) a poly A signal, and e) optionally an intron; and (3) a transgene providing (e.g., coding for) one or more RNA or protein products of interest.

In some aspects, the disclosure provides for a nucleic acid for use, wherein the nucleic acid encodes a therapeutic product operatively linked to a promoter or enhancer-promoter described herein.

In some aspects, the disclosure provides for a nucleic acid for use, wherein the nucleic acid encodes a HuPTMFabVEGFi, e.g., HuGlyFabVEGFi operatively linked to a promoter selected from the group consisting of: the CB7 promoter (a chicken β-actin promoter and CMV enhancer), cytomegalovirus (CMV) promoter, Rous sarcoma virus (RSV) promoter, MMT promoter, EF-1 alpha promoter, UB6 promoter, chicken beta-actin promoter, CAG promoter, RPE65 promoter and opsin promoter. In a specific embodiment, HuPTMFabVEGFi is operatively linked to the CB7 promoter.

In certain embodiments, provided herein are recombinant vectors that comprise one or more nucleic acids (e.g. polynucleotides). The nucleic acids may comprise DNA, RNA, or a combination of DNA and RNA. In certain embodiments, the DNA comprises one or more of the sequences selected from the group consisting of promoter sequences, the sequence encoding the therapeutic product of interest (the transgene, e.g., an anti-VEGF antigen-binding fragment), untranslated regions, and termination sequences. In certain embodiments, recombinant vectors provided herein comprise a promoter operably linked to the sequence encoding the therapeutic product of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161).

In certain embodiments, the recombinant vectors provided herein comprise modified mRNA encoding for the therapeutic product of interest (e.g., the transgene, for example, an anti-VEGF antigen-binding fragment moiety). In certain embodiments, provided herein is a modified mRNA encoding for an anti-VEGF antigen-binding fragment moiety. In certain embodiments, the recombinant vectors provided herein comprise a nucleotide sequence encoding for a therapeutic product that is an shRNA, siRNA, or miRNA.

In certain embodiments, the vectors provided herein comprise components that modulate protein delivery. In certain embodiments, the viral vectors provided herein comprise one or more signal peptides. Examples of signal peptides include, but is not limited to, VEGF-A signal peptide (SEQ ID NO: 5), fibulin-1 signal peptide (SEQ ID NO: 6), vitronectin signal peptide (SEQ ID NO: 7), complement Factor H signal peptide (SEQ ID NO: 8), opticin signal peptide (SEQ ID NO: 9), albumin signal peptide (SEQ ID NO: 22), chymotrypsinogen signal peptide (SEQ ID NO: 23), interleukin-2 signal peptide (SEQ ID NO: 24), and trypsinogen-2 signal peptide (SEQ ID NO: 25), mutant interleukin-2 signal peptide (SEQ ID NO: 55).

(a) Viral Vectors

In some embodiments, the viral vectors provided herein are AAV based viral vectors. In preferred embodiments, the viral vectors provided herein are AAV8 based viral vectors. In certain embodiments, the AAV8 based viral vectors provided herein retain tropism for retinal cells. In certain embodiments, the AAV-based vectors provided herein encode the AAV rep gene (required for replication) and/or the AAV cap gene (required for synthesis of the capsid proteins). Multiple AAV serotypes have been identified. In certain embodiments, AAV-based vectors provided herein comprise components from one or more serotypes of AAV. In certain embodiments, AAV based vectors provided herein comprise capsid components from one or more of AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In preferred embodiments, AAV based vectors provided herein comprise components from one or more of AAV8, AAV9, AAV10, AAV11, or AAVrh10 serotypes. In certain embodiments, the recombinant viral vectors provided herein are altered such that they are replication-deficient in humans. In certain embodiments, the recombinant viral vectors are hybrid vectors, e.g., an AAV vector placed into a “helpless” adenoviral vector. In certain embodiments, provided herein are recombinant viral vectors comprising a viral capsid from a first virus and viral envelope proteins from a second virus. In specific embodiments, the second virus is vesicular stomatitis virus (VSV). In more specific embodiments, the envelope protein is VSV-G protein.

Provided in particular embodiments are AAV8 vectors comprising a viral genome comprising an expression cassette for expression of the transgene, under the control of regulatory elements and flanked by ITRs and a viral capsid that has the amino acid sequence of the AAV8 capsid protein or is at least 95%, 96%, 97%, 98%, 99% or 99.9% identical to the amino acid sequence of the AAV8 capsid protein (SEQ ID NO: 48) while retaining the biological function of the AAV8 capsid. In certain embodiments, the encoded AAV8 capsid has the sequence of SEQ ID NO: 48 with 1, 2, 3, 4, 5, 6, 7, 8, 9, 10, 11, 12, 13, 14, 15, 16, 17, 18, 19, 21, 22, 23, 24, 25, 26, 27, 28, 29 or 30 amino acid substitutions and retaining the biological function of the AAV8 capsid.

In certain embodiments, the AAV that is used in the methods described herein is Anc80 or Anc80L65, as described in Zinn et al., 2015, Cell Rep. 12(6): 1056-1068, which is incorporated by reference in its entirety. In certain embodiments, the AAV that is used in the methods described herein comprises one of the following amino acid insertions: LGETTRP or LALGETTRP, as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is AAV.7m8, as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is any AAV disclosed in U.S. Pat. No. 9,585,971, such as AAV.PHP.B. In certain embodiments, the AAV that is used in the methods described herein is an AAV disclosed in any of the following patents and patent applications, each of which is incorporated herein by reference in its entirety: U.S. Pat. Nos. 7,906,111; 8,524,446; 8,999,678; 8,628,966; 8,927,514; 8,734,809; 9,284,357; 9,409,953; 9,169,299; 9,193,956; 9,458,517; and 9,587,282 US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

AAV8-based viral vectors are used in certain of the methods described herein. Nucleic acid sequences of AAV based viral vectors and methods of making recombinant AAV and AAV capsids are taught, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety. In one aspect, provided herein are AAV (e.g., AAV8)-based viral vectors encoding a transgene (e.g., an anti-VEGF antigen-binding fragment). In specific embodiments, provided herein are AAV8-based viral vectors encoding an anti-VEGF antigen-binding fragment. In more specific embodiments, provided herein are AAV8-based viral vectors encoding ranibizumab.

In certain embodiments, a single-stranded AAV (ssAAV) may be used supra. In certain embodiments, a self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

In certain embodiments, the viral vectors used in the methods described herein are adenovirus based viral vectors. A recombinant adenovirus vector may be used to transfer in the anti-VEGF antigen-binding fragment. The recombinant adenovirus can be a first generation vector, with an E1 deletion, with or without an E3 deletion, and with the expression cassette inserted into either deleted region. The recombinant adenovirus can be a second generation vector, which contains full or partial deletions of the E2 and E4 regions. A helper-dependent adenovirus retains only the adenovirus inverted terminal repeats and the packaging signal (phi). The transgene is inserted between the packaging signal and the 3′ITR, with or without stuffer sequences to keep the genome close to wild-type size of approx. 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

In a specific embodiment, a vector for use in the methods described herein is one that encodes an anti-VEGF antigen-binding fragment (e.g., ranibizumab) such that, upon introduction of the vector into a relevant cell (e.g., a retinal cell in vivo or in vitro), a glycosylated and or tyrosine sulfated variant of the anti-VEGF antigen-binding fragment is expressed by the cell. In a specific embodiment, the expressed anti-VEGF antigen-binding fragment comprises a glycosylation and/or tyrosine sulfation pattern.

(b) Therapeutic Product or Transgenes

The therapeutic products can be, for example, therapeutic proteins (for example, antibodies), therapeutic RNAs (for example, shRNAs, siRNAs, and miRNAs), or therapeutic aptamers.

In certain embodiments, the disclosure provides a pharmaceutical composition comprising recombinant AAV encoding a transgene. In some embodiments, provided herein are rAAV viral vectors encoding an anti-VEGF Fab or anti-VEGF antibody. In some embodiments, provided herein are rAAV8-based viral vectors encoding an anti-VEGF Fab or anti-VEGF antibody. In some embodiments, provided herein are rAAV8-based viral vectors encoding ranibizumab. In some embodiments, provided herein are rAAV viral vectors encoding Iduronidase (IDUA). In some embodiments, provided herein are rAAV9-based viral vectors encoding IDUA. In some embodiments, provided herein are rAAV viral vectors encoding Iduronate 2-Sulfatase (IDS). In some embodiments, provided herein are rAAV9-based viral vectors encoding IDS. In some embodiments, provided herein are rAAV viral vectors encoding a low-density lipoprotein receptor (LDLR). In some embodiments, provided herein are rAAV8-based viral vectors encoding LDLR. In some embodiments, provided herein are rAAV viral vectors encoding tripeptidyl peptidase 1 (TPP1) protein. In some embodiments, provided herein are rAAV9-based viral vectors encoding TPP1. In some embodiments, provided herein are rAAV viral vectors encoding microdystrophin protein. In some embodiments, provided herein are rAAV8-based viral vectors encoding microdystrophin. In some embodiments, provided herein are rAAV9-based viral vectors encoding microdystrophin. In some embodiments, provided herein are rAAV viral vectors encoding anti-kallikrein (anti-pKal) protein. In some embodiments, provided herein are rAAV8-based or rAAV9-based viral vectors encoding lanadelumab Fab or full-length antibody. In some embodiments, provided herein are rAAV viral vectors encoding human-alpha-sarcoglycan-gamma-sarcoglycan. In some embodiments, provided herein are rAAV viral vectors encoding huFollistatin344. In some embodiments, provided herein are rAAV viral vectors encoding human-alpha-sarcoglycan-gamma-sarcoglycan. In some embodiments, provided herein are rAAV viral vectors encoding CLN2. In some embodiments, provided herein are rAAV viral vectors encoding CLN3. In some embodiments, provided herein are rAAV viral vectors encoding CLN6. In some embodiments, provided herein are rAAV8-based or rAAV9-based viral vectors encoding human-alpha-sarcoglycan-gamma-sarcoglycan. In some embodiments, provided herein are rAAV8-based or rAAV9-based viral vectors encoding huFollistatin344. In some embodiments, provided herein are rAAV8-based or rAAV9-based viral vectors encoding human-alpha-sarcoglycan-gamma-sarcoglycan. In some embodiments, provided herein are rAAV8-based or rAAV9-based viral vectors encoding CLN2. In some embodiments, provided herein are rAAV8-based or rAAV9-based viral vectors encoding CLN3. In some embodiments, provided herein are rAAV8-based or rAAV9-based viral vectors encoding CLN6.

In certain embodiments, the therapeutic product (e.g., transgene) is: (1) anti-human vascular endothelial growth factor (hVEGF) antibody or aptamer; (2) an anti-hVEGF antigen-binding fragment; (3) anti-hVEGF antigen-binding fragment is a Fab, F(ab′)2, or single chain variable fragment (scFv); (4) Palmitoyl-Protein Thioesterase 1 (PPT1); (5) Tripeptidyl-Peptidase 1 (TPP1); (6) Battenin (CLN3); and (7) CLN6 Transmembrane ER Protein (CLN6).

In certain embodiments, the disclosure provides a pharmaceutical composition comprising recombinant AAV encoding a transgene. In some embodiments, provided herein are rAAV viral vectors encoding an anti-VEGF Fab or anti-VEGF antibody. In some embodiments, provided herein are rAAV8-based viral vectors encoding an anti-VEGF Fab or anti-VEGF antibody. In more embodiments, provided herein are rAAV8-based viral vectors encoding ranibizumab. In some embodiments, provided herein are rAAV viral vectors encoding Iduronidase (IDUA). In some embodiments, provided herein are rAAV9-based viral vectors encoding IDUA. In some embodiments, provided herein are rAAV viral vectors encoding Iduronate 2-Sulfatase (IDS). In some embodiments, provided herein are rAAV9-based viral vectors encoding IDS. In some embodiments, provided herein are rAAV viral vectors encoding a low-density lipoprotein receptor (LDLR). In some embodiments, provided herein are rAAV8-based viral vectors encoding LDLR. In some embodiments, provided herein are rAAV viral vectors encoding tripeptidyl peptidase 1 (TPP1) protein. In some embodiments, provided herein are rAAV9-based viral vectors encoding TPP1. In some embodiments, provided herein are rAAV viral vectors encoding microdystrophin protein. In some embodiments, provided herein are rAAV8-based viral vectors encoding microdystrophin. In some embodiments, provided herein are rAAV9-based viral vectors encoding microdystrophin. In some embodiments, provided herein are rAAV viral vectors encoding anti-kallikrein (anti-pKal) protein. In some embodiments, provided herein are rAAV8-based or rAAV9-based viral vectors encoding lanadelumab Fab or full-length antibody. In some embodiments, provided herein are rAAV viral vectors encoding human-alpha-sarcoglycan-gamma-sarcoglycan. In some embodiments, provided herein are rAAV viral vectors encoding huFollistatin344. In some embodiments, provided herein are rAAV viral vectors encoding human-alpha-sarcoglycan-gamma-sarcoglycan. In some embodiments, provided herein are rAAV viral vectors encoding CLN2. In some embodiments, provided herein are rAAV viral vectors encoding CLN3. In some embodiments, provided herein are rAAV viral vectors encoding CLN6. In some embodiments, provided herein are rAAV8-based or rAAV9-based viral vectors encoding human-alpha-sarcoglycan-gamma-sarcoglycan. In some embodiments, provided herein are rAAV8-based or rAAV9-based viral vectors encoding huFollistatin344. In some embodiments, provided herein are rAAV8-based or rAAV9-based viral vectors encoding human-alpha-sarcoglycan-gamma-sarcoglycan. In some embodiments, provided herein are rAAV8-based or rAAV9-based viral vectors encoding CLN2. In some embodiments, provided herein are rAAV8-based or rAAV9-based viral vectors encoding CLN3. In some embodiments, provided herein are rAAV8-based or rAAV9-based viral vectors encoding CLN6.

In certain embodiments, the vectors provided herein can be used for (1) the pathology of the eye associated with Batten-CLN1 and the therapeutic product is Palmitoyl-Protein Thioesterase 1 (PPT1); (2) the pathology of the eye associated with Batten-CLN2 and the therapeutic product is Tripeptidyl-Peptidase 1 (TPP1); (3) the pathology of the eye associated with Batten-CLN3 and the therapeutic product is Battenin (CLN3); (4) the pathology of the eye associated with Batten-CLN6 and the therapeutic product is CLN6 Transmembrane ER Protein (CLN6); (5) the pathology of the eye associated with Batten-CLN7 and the therapeutic product is Major Facilitator Superfamily Domain Containing 8 (MFSD8); and (6) the pathology of the eye associated with Batten-CLN1 and the therapeutic product is Palmitoyl-Protein Thioesterase 1 (PPT1).

In some embodiments, the HuPTMFabVEGFi, e.g., HuGlyFabVEGFi encoded by the transgene can include, but is not limited to an antigen-binding fragment of an antibody that binds to VEGF, such as bevacizumab; an anti-VEGF Fab moiety such as ranibizumab; or such bevacizumab or ranibizumab Fab moieties engineered to contain additional glycosylation sites on the Fab domain (e.g., see Courtois et al., 2016, mAbs8: 99-112 which is incorporated by reference herein in its entirety for it description of derivatives of bevacizumab that are hyperglycosylated on the Fab domain of the full length antibody).

In certain embodiments, the vectors provided herein encode an anti-VEGF antigen-binding fragment transgene. In specific embodiments, the anti-VEGF antigen-binding fragment transgene is controlled by appropriate expression control elements for expression in retinal cells: In certain embodiments, the anti-VEGF antigen-binding fragment transgene comprises bevacizumab Fab portion of the light and heavy chain cDNA sequences (SEQ ID Nos: 10 and 11, respectively). In certain embodiments, the anti-VEGF antigen-binding fragment transgene comprises ranibizumab light and heavy chain cDNA sequences (SEQ ID Nos: 12 and 13, respectively). In certain embodiments, the anti-VEGF antigen-binding fragment transgene encodes a bevacizumab Fab, comprising a light chain and a heavy chain of SEQ ID NOs: 3 and 4, respectively. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3. In certain embodiments, the anti-VEGF antigen-binding fragment transgene encodes an antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4. In certain embodiments, the anti-VEGF antigen-binding fragment transgene encodes a hyperglycosylated ranibizumab, comprising a light chain and a heavy chain of SEQ ID NOs: 1 and 2, respectively. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1. In certain embodiments, the anti-VEGF antigen-binding fragment transgene encodes an antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2.

In certain embodiments, the anti-VEGF antigen-binding fragment transgene encodes a hyperglycosylated bevacizumab Fab, comprising a light chain and a heavy chain of SEQ ID NOs: 3 and 4, with one or more of the following mutations: L118N (heavy chain), E195N (light chain), or Q160N or Q1605 (light chain). In certain embodiments, the anti-VEGF antigen-binding fragment transgene encodes a hyperglycosylated ranibizumab, comprising a light chain and a heavy chain of SEQ ID NOs: 1 and 2, with one or more of the following mutations: L118N (heavy chain), E195N (light chain), or Q160N or Q1605 (light chain). The sequences of the antigen-binding fragment transgene cDNAs may be found, for example, in Table 1. In certain embodiments, the sequence of the antigen-binding fragment transgene cDNAs is obtained by replacing the signal sequence of SEQ ID NOs: 10 and 11 or SEQ ID NOs: 12 and 13 with one or more signal sequences.

In certain embodiments, the anti-VEGF antigen-binding fragment transgene encodes an antigen-binding fragment and comprises the nucleotide sequences of the six bevacizumab CDRs. In certain embodiments, the anti-VEGF antigen-binding fragment transgene encodes an antigen-binding fragment and comprises the nucleotide sequences of the six ranibizumab CDRs. In certain embodiments, the anti-VEGF antigen-binding fragment transgene encodes an antigen-binding fragment comprising a heavy chain variable region comprising heavy chain CDRs 1-3 of ranibizumab (SEQ ID NOs: 20, 18, and 21). In certain embodiments, the anti-VEGF antigen-binding fragment transgene encodes an antigen-binding fragment comprising a light chain variable region comprising light chain CDRs 1-3 of ranibizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of bevacizumab (SEQ ID NOs: 17-19). In certain embodiments, the anti-VEGF antigen-binding fragment transgene encodes an antigen-binding fragment comprising a light chain variable region comprising light chain CDRs 1-3 of bevacizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of ranibizumab (SEQ ID NOs: 20, 18, and 21) and a light chain variable region comprising light chain CDRs 1-3 of ranibizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of bevacizumab (SEQ ID NOs: 17-19) and a light chain variable region comprising light chain CDRs 1-3 of bevacizumab (SEQ ID NOs: 14-16).

In certain embodiments, the anti-VEGF antigen-binding fragment transgene encodes an antigen-binding fragment comprising a light chain variable region comprising light chain CDRs 1-3 of SEQ ID NOs: 14-16, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16, wherein the second amino acid residue of the light chain CDR3 (i.e., the second Q in QQYSTVPWTF (SEQ ID NO. 16)) is not acetylated. In a specific embodiment, the anti-VEGF antigen-binding fragment transgene encodes an antigen-binding fragment comprising a light chain variable region comprising light chain CDRs 1-3 of SEQ ID NOs: 14-16, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a heavy chain variable region comprising heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a heavy chain variable region comprising heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a heavy chain variable region comprising heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated. In a specific embodiment, the anti-VEGF antigen-binding fragment transgene encodes an antigen-binding fragment comprising a heavy chain variable region comprising heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a light chain variable region comprising light chain CDRs 1-3 of SEQ ID NOs: 14-16 and a heavy chain variable region comprising heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu), and wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16 and a heavy chain variable region comprising heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16 and a heavy chain variable region comprising heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) is not acetylated, and wherein the last amino acid residue of the heavy chain CDR1 (i.e., the N in GYDFTHYGMN (SEQ ID NO. 20)) is not acetylated. In a specific embodiment, the antigen-binding fragment comprises a heavy chain CDR1 of SEQ ID NO. 20, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated; and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, and transgenes encoding such antigen-VEGF antigen-binding fragments, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. The anti-VEGF antigen-binding fragments and transgenes provided herein can be used in any method according to the invention described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, and transgenes encoding such antigen-VEGF antigen-binding fragments, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated. The anti-VEGF antigen-binding fragments and transgenes provided herein can be used in any method according to the invention described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, and transgenes encoding such antigen-VEGF antigen-binding fragments, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated, and the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated; and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. The anti-VEGF antigen-binding fragments and transgenes provided herein can be used in any method according to the invention described herein. In a preferred embodiment, the chemical modification(s) or lack of chemical modification(s) (as the case may be) described herein is determined by mass spectrometry.

TABLE 1
Exemplary Sequences

| SEQ | | |
|---|---|---|
| ID | | |
| NO: | Description | Sequence |
| 1 | Ranibizumab | DIQLTQSPSSLSASVGDRVTITCSASQDISNYLNWYQQKPGKAPKVLIYFTSSLH |
| | Fab Amino | SGVPSRFSGSGSGTDFTLTISSLQPEDFATYYCQQYSTVPWTFGQGTKVEIKRTV |
| | Acid Sequence | AAPSVFIFPPSDEQLKSGTASVVCLLNNFYPREAKVQWKVDNALQSGNSQESVTE |
| | (Light chain) | QDSKDSTYSLSSTLTLSKADYEKHKVYACEVTHQGLSSPVTKSENRGEC |
| 2 | Ranibizumab | EVQLVESGGGLVQPGGSLRLSCAASGYDFTHYGMNWVRQAPGKGLEWVGWINTYT |
| | Fab Amino | GEPTYAADFKRRFTFSLDTSKSTAYLQMNSLRAEDTAVYYCAKYPYYYGTSHWYF |
| | Acid Sequence | DVWGQGTLVTVSSASTKGPSVFPLAPSSKSTSGGTAALGCLVKDYFPEPVTVSWN |
| | (Heavy chain) | SGALTSGVHTFPAVLQSSGLYSLSSVVTVPSSSLGTQTYICNVNHKPSNTKVDKK |
| | | VEPKSCDKTHL |
| 3 | Bevacizumab | DIQMTQSPSSLSASVGDRVTITCSASQDISNYLNWYQQKPGKAPKVLIYFTSSLH |
| | Fab Amino | SGVPSRFSGSGSGTDFTLTISSLQPEDFATYYCQQYSTVPWTFGQGTKVEIKRTV |
| | Acid Sequence | AAPSVFIFPPSDEQLKSGTASVVCLLNNFYPREAKVQWKVDNALQSGNSQESVTE |
| | (Light chain) | QDSKDSTYSLSSTLTLSKADYEKHKVYACEVTHQGLSSPVTKSENRGEC |
| 4 | Bevacizumab | EVQLVESGGGLVQPGGSLRLSCAASGYTFTNYGMNWVRQAPGKGLEWVGWINTYT |
| | Fab Amino | GEPTYAADFKRRFTFSLDTSKSTAYLQMNSLRAEDTAVYYCAKYPHYYGSSHWYF |
| | Acid Sequence | DVWGQGTLVTVSSASTKGPSVFPLAPSSKSTSGGTAALGCLVKDYFPEPVTVSWN |
| | (Heavy chain) | SGALTSGVHTFPAVLQSSGLYSLSSVVTVPSSSLGTQTYICNVNHKPSNTKVDKK |
| | | VEPKSCDKTHL |
| 5 | VEGF-A signal | MNFLLSWVHW SLALLLYLHH AKWSQA |
| | peptide | |
| 6 | Fibulin-1 | MERAAPSRRV PLPLLLLGGL ALLAAGVDA |
| | signal peptide | |
| 7 | Vitronectin | MAPLRPLLIL ALLAWVALA |
| | signal peptide | |
| 8 | Complement | MRLLAKIICLMLWAICVA |
| | Factor H signal | |
| | peptide | |
| 9 | Opticin signal | MRLLAFLSLL ALVLQETGT |
| | peptide | |
| 10 | Bevacizumab | gctagcgcca ccatgggctg gtcctgcatc atcctgttcc tggtggccac |
| | CDNA | cgccaccggc gtgcactccg acatccagat gacccagtcc ccctcctccc |
| | (Light chain) | tgtccgcctc cgtgggcgac cgggtgacca tcacctgctc cgcctcccag |
| | | gacatctcca actacctgaa ctggtaccag cagaagcccg gcaaggcccc |
| | | caaggtgctg atctacttca cctcctccct gcactccggc gtgccctccc |
| | | ggttctccgg ctccggctcc ggcaccgact tcaccctgac catctcctcc |
| | | ctgcagcccg aggacttcgc cacctactac tgccagcagt actccaccgt |
| | | gccctggacc ttcggccagg gcaccaaggt ggagatcaag cggaccgtgg |
| | | ccgccccctc cgtgttcatc ttccccccct ccgacgagca gctgaagtcc |
| | | ggcaccgcct ccgtggtgtg cctgctgaac aacttctacc cccgggaggc |
| | | caaggtgcag tggaaggtgg acaacgccct gcagtccggc aactcccagg |
| | | agtccgtgac cgagcaggac tccaaggact ccacctactc cctgtcctcc |
| | | accctgaccc tgtccaaggc cgactacgag aagcacaagg tgtacgcctg |
| | | cgaggtgacc caccagggcc tgtcctcccc cgtgaccaag tccttcaacc |
| | | ggggcgagtg ctgagcggcc gcctcgag |
| 11 | Bevacizumab | gctagcgcca ccatgggctg gtcctgcatc atcctgttcc tggtggccac |
| | CDNA (Heavy | cgccaccggc gtgcactccg aggtgcagct ggtggagtcc ggcggcggcc |
| | chain) | tggtgcagcc cggcggctcc ctgcggctgt cctgcgccgc ctccggctac |
| | | accttcacca actacggcat gaactgggtg cggcaggccc ccggcaaggg |
| | | cctggagtgg gtgggctgga tcaacaccta caccggcgag cccacctacg |
| | | ccgccgactt caagcggcgg ttcaccttct ccctggacac ctccaagtcc |
| | | accgcctacc tgcagatgaa ctccctgcgg gccgaggaca ccgccgtgta |
| | | ctactgcgcc aagtaccccc actactacgg ctcctcccac tggtacttcg |
| | | acgtgtgggg ccagggcacc ctggtgaccg tgtcctccgc ctccaccaag |
| | | ggcccctccg tgttccccct ggccccctcc tccaagtcca cctccggcgg |
| | | caccgccgcc ctgggctgcc tggtgaagga ctacttcccc gagcccgtga |
| | | ccgtgtcctg gaactccggc gccctgacct ccggcgtgca caccttcccc |
| | | gccgtgctgc agtcctccgg cctgtactcc ctgtcctccg tggtgaccgt |
| | | gccctcctcc tccctgggca cccagaccta catctgcaac gtgaaccaca |
| | | agccctccaa caccaaggtg gacaagaagg tggagcccaa gtcctgcgac |
| | | aagacccaca cctgcccccc ctgccccgcc cccgagctgc tgggcggccc |
| | | ctccgtgttc ctgttccccc ccaagcccaa ggacaccctg atgatctccc |
| | | ggacccccga ggtgacctgc gtggtggtgg acgtgtccca cgaggacccc |
| | | gaggtgaagt tcaactggta cgtggacggc gtggaggtgc acaacgccaa |
| | | gaccaagccc cgggaggagc agtacaactc cacctaccgg gtggtgtccg |
| | | tgctgaccgt gctgcaccag gactggctga acggcaagga gtacaagtgc |
| | | aaggtgtcca acaaggccct gcccgccccc atcgagaaga ccatctccaa |
| | | ggccaagggc cagccccggg agccccaggt gtacaccctg cccccctccc |
| | | gggaggagat gaccaagaac caggtgtccc tgacctgcct ggtgaagggc |
| | | ttctacccct ccgacatcgc cgtggagtgg gagtccaacg gccagcccga |
| | | gaacaactac aagaccaccc cccccgtgct ggactccgac ggctccttct |
| | | tcctgtactc caagctgacc gtggacaagt cccggtggca gcagggcaac |
| | | gtgttctcct gctccgtgat gcacgaggcc ctgcacaacc actacaccca |
| | | gaagtccctg tccctgtccc ccggcaagtg agcggccgcc |
| 12 | Ranibizumab | gagctccatg gagtttttca aaaagacggc acttgccgca ctggttatgg |
| | cDNA (Light | gttttagtgg tgcagcattg gccgatatcc agctgaccca gagcccgagc |
| | chain | agcctgagcg caagcgttgg tgatcgtgtt accattacct gtagcgcaag |
| | comprising a | ccaggatatt agcaattatc tgaattggta tcagcagaaa ccgggtaaag |
| | signal | caccgaaagt tctgatttat tttaccagca gcctgcatag cggtgttccg |
| | sequence) | agccgtttta gcggtagcgg tagtggcacc gattttaccc tgaccattag |
| | | cagcctgcag ccggaagatt ttgcaaccta ttattgtcag cagtatagca |
| | | ccgttccgtg gacctttggt cagggcacca aagttgaaat taaacgtacc |
| | | gttgcagcac cgagcgtttt tatttttccg cctagtgatg aacagctgaa |
| | | aagcggcacc gcaagcgttg tttgtctgct gaataatttt tatccgcgtg |
| | | aagcaaaagt gcagtggaaa gttgataatg cactgcagag cggtaatagc |
| | | caagaaagcg ttaccgaaca ggatagcaaa gatagcacct atagcctgag |
| | | cagcaccctg accctgagca aagcagatta tgaaaaacac aaagtgtatg |
| | | cctgcgaagt tacccatcag ggtctgagca gtccggttac caaaagtttt |
| | | aatcgtggcg aatgctaata gaagcttggt acc |
| 13 | Ranibizumab | gagctcatat gaaatacctg ctgccgaccg ctgctgctgg tctgctgctc |
| | cDNA (Heavy | ctcgctgccc agccggcgat ggccgaagtt cagctggttg aaagcggtgg |
| | chain | tggtctggtt cagcctggtg gtagcctgcg tctgagctgt gcagcaagcg |
| | comprising a | gttatgattt tacccattat ggtatgaatt gggttcgtca ggcaccgggt |
| | signal | aaaggtctgg aatgggttgg ttggattaat acctataccg gtgaaccgac |
| | sequence) | ctatgcagca gattttaaac gtcgttttac ctttagcctg gataccagca |
| | | aaagcaccgc atatctgcag atgaatagcc tgcgtgcaga agataccgca |
| | | gtttattatt gtgccaaata tccgtattac tatggcacca gccactggta |
| | | tttcgatgtt tggggtcagg gcaccctggt taccgttagc agcgcaagca |
| | | ccaaaggtcc gagcgttttt ccgctggcac cgagcagcaa aagtaccagc |
| | | ggtggcacag cagcactggg ttgtctggtt aaagattatt ttccggaacc |
| | | ggttaccgtg agctggaata gcggtgcact gaccagcggt gttcatacct |
| | | ttccggcagt tctgcagagc agcggtctgt atagcctgag cagcgttgtt |
| | | accgttccga gcagcagcct gggcacccag acctatattt gtaatgttaa |
| | | tcataaaccg agcaatacca aagtggataa aaaagttgag ccgaaaagct |
| | | gcgataaaac ccatctgtaa tagggtacc |
| 14 | Bevacizumab | SASQDISNYLN |
| | and | |
| | Ranibizumab | |
| | Light Chain | |
| | CDR1 | |
| 15 | Bevacizumab | FTSSLHS |
| | and | |
| | Ranibizumab | |
| | Light Chain | |
| | CDR2 | |
| 16 | Bevacizumab | QQYSTVPWT |
| | and | |
| | Ranibizumab | |
| | Light Chain | |
| | CDR3 | |
| 17 | Bevacizumab | GYTFTNYGMN |
| | Heavy Chain | |
| | CDR1 | |
| 18 | Bevacizumab | WINTYTGEPTYAADEKR |
| | and | |
| | Ranibizumab | |
| | Heavy Chain | |
| | CDR2 | |
| 19 | Bevacizumab | YPHYYGSSHWYFDV |
| | Heavy Chain | |
| | CDR3 | |
| 20 | Ranibizumab | GYDFTHYGMN |
| | Heavy Chain | |
| | CDR1 | |
| 21 | Ranibizumab | YPYYYGTSHWYFDV |
| | Heavy Chain | |
| | CDR3 | |
| 22 | Albumin signal | MKWVTFISLLFLESSAYS |
| | peptide | |
| 23 | Chymotrypsino | MAFLWLLSCWALLGTTFG |
| | gen signal | |
| | peptide | |
| 24 | Interleukin-2 | MYRMQLLSCIALILALVINS |
| | signal peptide | |
| 25 | Trypsinogen-2 | MNLLLILTFVAAAVA |
| | signal peptide | |
| 26 | F2A site | LLNFDLLKLAGDVESNPGP |
| 27 | T2A site | (GSG)EGRGSLLTCGDVEENPGP |
| 28 | P2A site | (GSG)ATNFSLLKQAGDVEENPGP |
| 29 | E2A site | (GSG)QCTNYALLKLAGDVESNPGP |
| 30 | F2A site | (GSG)VKQTLNFDLLKLAGDVESNPGP |
| 31 | Furin linker | RKRR |
| 32 | Furin linker | RRRR |
| 33 | Furin linker | RRKR |
| 34 | Furin linker | RKKR |
| 35 | Furin linker | R-X-K/R-R |
| 36 | Furin linker | RXKR |
| 37 | Furin linker | RXRR |
| 38 | Ranibizumab | MDIQLTQSPSSLSASVGDRVTITCSASQDISNYLNWYQQKPGKAPKVLIYFTSSL |
| | Fab amino acid | HSGVPSRFSGSGSGTDFTLTISSLQPEDFATYYCQQYSTVPWTFGQGTKVEIKRT |
| | sequence (Light | VAAPSVFIFPPSDEQLKSGTASVVCLLNNFYPREAKVQWKVDNALQSGNSQESVT |
| | chain) | EQDSKDSTYSLSSTLTLSKADYEKHKVYACEVTHQGLSSPVTKSENRGEC |
| 39 | Ranibizumab | MEVQLVESGGGLVQPGGSLRLSCAASGYDFTHYGMNWVRQAPGKGLEWVGWINTY |
| | Fab amino acid | TGEPTYAADFKRRFTFSLDTSKSTAYLQMNSLRAEDTAVYYCAKYPYYYGTSHWY |
| | sequence | FDVWGQGTLVTVSSASTKGPSVFPLAPSSKSTSGGTAALGCLVKDYFPEPVTVSW |
| | (Heavy chain) | NSGALTSGVHTFPAVLQSSGLYSLSSVVTVPSSSLGTQTYICNVNHKPSNTKVDK |
| | | KVEPKSCDKTHLRKRR |
| 40 | Ranibizumab | MEVQLVESGGGLVQPGGSLRLSCAASGYDFTHYGMNWVRQAPGKGLEWVGWINTY |
| | Fab amino acid | TGEPTYAADFKRRFTFSLDTSKSTAYLQMNSLRAEDTAVYYCAKYPYYYGTSHWY |
| | sequence | FDVWGQGTLVTVSSASTKGPSVFPLAPSSKSTSGGTAALGCLVKDYFPEPVTVSW |
| | (Heavy chain) | NSGALTSGVHTFPAVLQSSGLYSLSSVVTVPSSSLGTQTYICNVNHKPSNTKVDK |
| | | KVEPKSCDKTHL |
| 41 | AAV1 | MAADGYLPDWLEDNLSEGIREWWDLKPGAPKPKANQQKQDDGRGLVLPGYKYLGP |
| | | FNGLDKGEPVNAADAAALEHDKAYDQQLKAGDNPYLRYNHADAEFQERLQEDTSF |
| | | GGNLGRAVFQAKKRVLEPLGLVEEGAKTAPGKKRPVEQSPQEPDSSSGIGKTGQQ |
| | | PAKKRLNFGQTGDSESVPDPQPLGEPPATPAAVGPTTMASGGGAPMADNNEGADG |
| | | VGNASGNWHCDSTWLGDRVITTSTRTWALPTYNNHLYKQISSASTGASNDNHYFG |
| | | YSTPWGYFDFNRFHCHFSPRDWQRLINNNWGFRPKRLNFKLFNIQVKEVTTNDGV |
| | | TTIANNLTSTVQVFSDSEYQLPYVLGSAHQGCLPPFPADVFMIPQYGYLTLNNGS |
| | | QAVGRSSFYCLEYFPSQMLRTGNNFTFSYTFEEVPFHSSYAHSQSLDRLMNPLID |
| | | QYLYYLNRTQNQSGSAQNKDLLESRGSPAGMSVQPKNWLPGPCYRQQRVSKTKTD |
| | | NNNSNFTWTGASKYNLNGRESIINPGTAMASHKDDEDKFFPMSGVMIFGKESAGA |
| | | SNTALDNVMITDEEEIKATNPVATERFGTVAVNFQSSSTDPATGDVHAMGALPGM |
| | | VWQDRDVYLQGPIWAKIPHTDGHFHPSPLMGGFGLKNPPPQILIKNTPVPANPPA |
| | | EFSATKFASFITQYSTGQVSVEIEWELQKENSKRWNPEVQYTSNYAKSANVDFTV |
| | | DNNGLYTEPRPIGTRYLTRPL |
| 42 | AAV2 | MAADGYLPDWLEDTLSEGIRQWWKLKPGPPPPKPAERHKDDSRGLVLPGYKYLGP |
| | | FNGLDKGEPVNEADAAALEHDKAYDRQLDSGDNPYLKYNHADAEFQERLKEDTSF |
| | | GGNLGRAVFQAKKRVLEPLGLVEEPVKTAPGKKRPVEHSPVEPDSSSGTGKAGQQ |
| | | PARKRLNFGQTGDADSVPDPQPLGQPPAAPSGLGTNTMATGSGAPMADNNEGADG |
| | | VGNSSGNWHCDSTWMGDRVITTSTRTWALPTYNNHLYKQISSQSGASNDNHYFGY |
| | | STPWGYFDFNRFHCHFSPRDWQRLINNNWGFRPKRLNFKLFNIQVKEVTQNDGTT |
| | | TIANNLTSTVQVFTDSEYQLPYVLGSAHQGCLPPFPADVFMVPQYGYLTLNNGSQ |
| | | AVGRSSFYCLEYFPSQMLRTGNNFTFSYTFEDVPFHSSYAHSQSLDRLMNPLIDQ |
| | | YLYYLSRTNTPSGTTTQSRLQFSQAGASDIRDQSRNWLPGPCYRQQRVSKTSADN |
| | | NNSEYSWTGATKYHLNGRDSLVNPGPAMASHKDDEEKFFPQSGVLIFGKQGSEKT |
| | | NVDIEKVMITDEEEIRTTNPVATEQYGSVSTNLQRGNRQAATADVNTQGVLPGMV |
| | | WQDRDVYLQGPIWAKIPHTDGHFHPSPLMGGFGLKHPPPQILIKNTPVPANPSTT |
| | | FSAAKFASFITQYSTGQVSVEIEWELQKENSKRWNPEIQYTSNYNKSVNVDFTVD |
| | | TNGVYSEPRPIGTRYLTRNL |
| 43 | AAV3-3 | MAADGYLPDWLEDNLSEGIREWWALKPGVPQPKANQQHQDNRRGLVLPGYKYLGP |
| | | GNGLDKGEPVNEADAAALEHDKAYDQQLKAGDNPYLKYNHADAEFQERLQEDTSF |
| | | GGNLGRAVFQAKKRILEPLGLVEEAAKTAPGKKGAVDQSPQEPDSSSGVGKSGKQ |
| | | PARKRLNFGQTGDSESVPDPQPLGEPPAAPTSLGSNTMASGGGAPMADNNEGADG |
| | | VGNSSGNWHCDSQWLGDRVITTSTRTWALPTYNNHLYKQISSQSGASNDNHYFGY |
| | | STPWGYFDFNRFHCHFSPRDWQRLINNNWGFRPKKLSFKLFNIQVRGVTQNDGTT |
| | | TIANNLTSTVQVFTDSEYQLPYVLGSAHQGCLPPFPADVEMVPQYGYLTLNNGSQ |
| | | AVGRSSFYCLEYFPSQMLRTGNNFQFSYTFEDVPFHSSYAHSQSLDRLMNPLIDQ |
| | | YLYYLNRTQGTTSGTTNQSRLLFSQAGPQSMSLQARNWLPGPCYRQQRLSKTAND |
| | | NNNSNFPWTAASKYHLNGRDSLVNPGPAMASHKDDEEKFFPMHGNLIFGKEGTTA |
| | | SNAELDNVMITDEEEIRTTNPVATEQYGTVANNLQSSNTAPTTGTVNHQGALPGM |
| | | VWQDRDVYLQGPIWAKIPHTDGHFHPSPLMGGFGLKHPPPQIMIKNTPVPANPPT |
| | | TFSPAKFASFITQYSTGQVSVEIEWELQKENSKRWNPEIQYTSNYNKSVNVDFTV |
| | | DTNGVYSEPRPIGTRYLTRNL |
| 44 | AAV4-4 | MTDGYLPDWLEDNLSEGVREWWALQPGAPKPKANQQHQDNARGLVLPGYKYLGPG |
| | | NGLDKGEPVNAADAAALEHDKAYDQQLKAGDNPYLKYNHADAEFQQRLQGDTSFG |
| | | GNLGRAVFQAKKRVLEPLGLVEQAGETAPGKKRPLIESPQQPDSSTGIGKKGKQP |
| | | AKKKLVFEDETGAGDGPPEGSTSGAMSDDSEMRAAAGGAAVEGGQGADGVGNASG |
| | | DWHCDSTWSEGHVTTTSTRTWVLPTYNNHLYKRLGESLQSNTYNGFSTPWGYFDF |
| | | NRFHCHFSPRDWQRLINNNWGMRPKAMRVKIFNIQVKEVTTSNGETTVANNLTST |
| | | VQIFADSSYELPYVMDAGQEGSLPPFPNDVFMVPQYGYCGLVTGNTSQQQTDRNA |
| | | FYCLEYFPSQMLRTGNNFEITYSFEKVPFHSMYAHSQSLDRLMNPLIDQYLWGLQ |
| | | STTTGTTLNAGTATTNFTKLRPTNFSNFKKNWLPGPSIKQQGFSKTANQNYKIPA |
| | | TGSDSLIKYETHSTLDGRWSALTPGPPMATAGPADSKFSNSQLIFAGPKQNGNTA |
| | | TVPGTLIFTSEEELAATNATDTDMWGNLPGGDQSNSNLPTVDRLTALGAVPGMVW |
| | | QNRDIYYQGPIWAKIPHTDGHFHPSPLIGGFGLKHPPPQIFIKNTPVPANPATTE |
| | | SSTPVNSFITQYSTGQVSVQIDWEIQKERSKRWNPEVQFTSNYGQQNSLLWAPDA |
| | | AGKYTEPRAIGTRYLTHHL |
| 45 | AAV5 | MSFVDHPPDWLEEVGEGLREFLGLEAGPPKPKPNQQHQDQARGLVLPGYNYLGPG |
| | | NGLDRGEPVNRADEVAREHDISYNEQLEAGDNPYLKYNHADAEFQEKLADDTSFG |
| | | GNLGKAVFQAKKRVLEPFGLVEEGAKTAPTGKRIDDHFPKRKKARTEEDSKPSTS |
| | | SDAEAGPSGSQQLQIPAQPASSLGADTMSAGGGGPLGDNNQGADGVGNASGDWHC |
| | | DSTWMGDRVVTKSTRTWVLPSYNNHQYREIKSGSVDGSNANAYFGYSTPWGYFDF |
| | | NRFHSHWSPRDWQRLINNYWGFRPRSLRVKIFNIQVKEVTVQDSTTTIANNLTST |
| | | VQVFTDDDYQLPYVVGNGTEGCLPAFPPQVFTLPQYGYATLNRDNTENPTERSSF |
| | | FCLEYFPSKMLRTGNNFEFTYNFEEVPFHSSFAPSQNLFKLANPLVDQYLYRFVS |
| | | TNNTGGVQFNKNLAGRYANTYKNWFPGPMGRTQGWNLGSGVNRASVSAFATTNRM |
| | | ELEGASYQVPPQPNGMTNNLQGSNTYALENTMIFNSQPANPGTTATYLEGNMLIT |
| | | SESETQPVNRVAYNVGGQMATNNQSSTTAPATGTYNLQEIVPGSVWMERDVYLQG |
| | | PIWAKIPETGAHFHPSPAMGGFGLKHPPPMMLIKNTPVPGNITSFSDVPVSSFIT |
| | | QYSTGQVTVEMEWELKKENSKRWNPEIQYTNNYNDPQFVDFAPDSTGEYRTTRPI |
| | | GTRYLTRPL |
| 46 | AAV6 | MAADGYLPDWLEDNLSEGIREWWDLKPGAPKPKANQQKQDDGRGLVLPGYKYLGP |
| | | FNGLDKGEPVNAADAAALEHDKAYDQQLKAGDNPYLRYNHADAEFQERLQEDTSF |
| | | GGNLGRAVFQAKKRVLEPFGLVEEGAKTAPGKKRPVEQSPQEPDSSSGIGKTGQQ |
| | | PAKKRLNFGQTGDSESVPDPQPLGEPPATPAAVGPTTMASGGGAPMADNNEGADG |
| | | VGNASGNWHCDSTWLGDRVITTSTRTWALPTYNNHLYKQISSASTGASNDNHYFG |
| | | YSTPWGYFDFNRFHCHFSPRDWQRLINNNWGFRPKRLNFKLFNIQVKEVTTNDGV |
| | | TTIANNLTSTVQVFSDSEYQLPYVLGSAHQGCLPPFPADVEMIPQYGYLTLNNGS |
| | | QAVGRSSFYCLEYFPSQMLRTGNNFTFSYTFEDVPFHSSYAHSQSLDRLMNPLID |
| | | QYLYYLNRTQNQSGSAQNKDLLFSRGSPAGMSVQPKNWLPGPCYRQQRVSKTKTD |
| | | NNNSNFTWTGASKYNLNGRESIINPGTAMASHKDDKDKFFPMSGVMIFGKESAGA |
| | | SNTALDNVMITDEEEIKATNPVATERFGTVAVNLQSSSTDPATGDVHVMGALPGM |
| | | VWQDRDVYLQGPIWAKIPHTDGHFHPSPLMGGFGLKHPPPQILIKNTPVPANPPA |
| | | EFSATKFASFITQYSTGQVSVEIEWELQKENSKRWNPEVQYTSNYAKSANVDFTV |
| | | DNNGLYTEPRPIGTRYLTRPL |
| 47 | AAV7 | MAADGYLPDWLEDNLSEGIREWWDLKPGAPKPKANQQKQDNGRGLVLPGYKYLGP |
| | | FNGLDKGEPVNAADAAALEHDKAYDQQLKAGDNPYLRYNHADAEFQERLQEDTSF |
| | | GGNLGRAVFQAKKRVLEPLGLVEEGAKTAPAKKRPVEPSPQRSPDSSTGIGKKGQ |
| | | QPARKRLNFGQTGDSESVPDPQPLGEPPAAPSSVGSGTVAAGGGAPMADNNEGAD |
| | | GVGNASGNWHCDSTWLGDRVITTSTRTWALPTYNNHLYKQISSETAGSTNDNTYF |
| | | GYSTPWGYFDFNRFHCHFSPRDWQRLINNNWGFRPKKLRFKLFNIQVKEVTTNDG |
| | | VTTIANNLTSTIQVFSDSEYQLPYVLGSAHQGCLPPFPADVEMIPQYGYLTLNNG |
| | | SQSVGRSSFYCLEYFPSQMLRTGNNFEFSYSFEDVPFHSSYAHSQSLDRLMNPLI |
| | | DQYLYYLARTQSNPGGTAGNRELQFYQGGPSTMAEQAKNWLPGPCFRQQRVSKTL |
| | | DQNNNSNFAWTGATKYHLNGRNSLVNPGVAMATHKDDEDRFFPSSGVLIFGKTGA |
| | | TNKTTLENVLMTNEEEIRPTNPVATEEYGIVSSNLQAANTAAQTQVVNNQGALPG |
| | | MVWQNRDVYLQGPIWAKIPHTDGNFHPSPLMGGFGLKHPPPQILIKNTPVPANPP |
| | | EVFTPAKFASFITQYSTGQVSVEIEWELQKENSKRWNPEIQYTSNFEKQTGVDFA |
| | | VDSQGVYSEPRPIGTRYLTRNL |
| 48 | AAV8 | MAADGYLPDWLEDNLSEGIREWWALKPGAPKPKANQQKQDDGRGLVLPGYKYLGP |
| | | FNGLDKGEPVNAADAAALEHDKAYDQQLQAGDNPYLRYNHADAEFQERLQEDTSF |
| | | GGNLGRAVFQAKKRVLEPLGLVEEGAKTAPGKKRPVEPSPQRSPDSSTGIGKKGQ |
| | | QPARKRLNFGQTGDSESVPDPQPLGEPPAAPSGVGPNTMAAGGGAPMADNNEGAD |
| | | GVGSSSGNWHCDSTWLGDRVITTSTRTWALPTYNNHLYKQISNGTSGGATNDNTY |
| | | FGYSTPWGYFDFNRFHCHFSPRDWQRLINNNWGFRPKRLSFKLFNIQVKEVTQNE |
| | | GTKTIANNLTSTIQVFTDSEYQLPYVLGSAHQGCLPPFPADVFMIPQYGYLTLNN |
| | | GSQAVGRSSFYCLEYFPSQMLRTGNNFQFTYTFEDVPFHSSYAHSQSLDRLMNPL |
| | | IDQYLYYLSRTQTTGGTANTQTLGFSQGGPNTMANQAKNWLPGPCYRQQRVSTTT |
| | | GQNNNSNFAWTAGTKYHLNGRNSLANPGIAMATHKDDEERFFPSNGILIFGKQNA |
| | | ARDNADYSDVMLTSEEEIKTTNPVATEEYGIVADNLQQQNTAPQIGTVNSQGALP |
| | | GMVWQNRDVYLQGPIWAKIPHTDGNFHPSPLMGGFGLKHPPPQILIKNTPVPADP |
| | | PTTFNQSKLNSFITQYSTGQVSVEIEWELQKENSKRWNPEIQYTSNYYKSTSVDF |
| | | AVNTEGVYSEPRPIGTRYLTRNL |
| 49 | hu31 | MAADGYLPDWLEDTLSEGIRQWWKLKPGPPPPKPAERHKDDSRGLVLPGYKYLGP |
| | | GNGLDKGEPVNAADAAALEHDKAYDQQLKAGDNPYLKYNHADAEFQERLKEDTSF |
| | | GGNLGRAVFQAKKRLLEPLGLVEEAAKTAPGKKRPVEQSPQEPDSSAGIGKSGSQ |
| | | PAKKKLNFGQTGDTESVPDPQPIGEPPAAPSGVGSLTMASGGGAPVADNNEGADG |
| | | VGSSSGNWHCDSQWLGDRVITTSTRTWALPTYNNHLYKQISNSTSGGSSNDNAYF |
| | | GYSTPWGYFDFNRFHCHFSPRDWQRLINNNWGFRPKRLNFKLFNIQVKEVTDNNG |
| | | VKTIANNLTSTVQVFTDSDYQLPYVLGSAHEGCLPPFPADVFMIPQYGYLTLNDG |
| | | GQAVGRSSFYCLEYFPSQMLRTGNNFQFSYEFENVPFHSSYAHSQSLDRLMNPLI |
| | | DQYLYYLSKTINGSGQNQQTLKFSVAGPSNMAVQGRNYIPGPSYRQQRVSTTVTQ |
| | | NNNSEFAWPGASSWALNGRNSLMNPGPAMASHKEGEDRFFPLSGSLIFGKQGTGR |
| | | DNVDADKVMITNEEEIKTTNPVATESYGQVATNHQSAQAQAQTGWVQNQGILPGM |
| | | VWQDRDVYLQGPIWAKIPHTDGNFHPSPLMGGFGMKHPPPQILIKNTPVPADPPT |
| | | AFNKDKLNSFITQYSTGQVSVEIEWELQKENSKRWNPEIQYTSNYYKSNNVEFAV |
| | | STEGVYSEPRPIGTRYLTRNL |
| 50 | hu32 | MAADGYLPDWLEDTLSEGIRQWWKLKPGPPPPKPAERHKDDSRGLVLPGYKYLGP |
| | | GNGLDKGEPVNAADAAALEHDKAYDQQLKAGDNPYLKYNHADAEFQERLKEDTSF |
| | | GGNLGRAVFQAKKRLLEPLGLVEEAAKTAPGKKRPVEQSPQEPDSSAGIGKSGSQ |
| | | PAKKKLNFGQTGDTESVPDPQPIGEPPAAPSGVGSLTMASGGGAPVADNNEGADG |
| | | VGSSSGNWHCDSQWLGDRVITTSTRTWALPTYNNHLYKQISNSTSGGSSNDNAYF |
| | | GYSTPWGYFDENRFHCHFSPRDWQRLINNNWGFRPKRLNFKLFNIQVKEVTDNNG |
| | | VKTIANNLTSTVQVFTDSDYQLPYVLGSAHEGCLPPFPADVFMIPQYGYLTLNDG |
| | | SQAVGRSSFYCLEYFPSQMLRTGNNFQFSYEFENVPFHSSYAHSQSLDRLMNPLI |
| | | DQYLYYLSKTINGSGQNQQTLKFSVAGPSNMAVQGRNYIPGPSYRQQRVSTTVTQ |
| | | NNNSEFAWPGASSWALNGRNSLMNPGPAMASHKEGEDRFFPLSGSLIFGKQGTGR |
| | | DNVDADKVMITNEEEIKTTNPVATESYGQVATNHQSAQAQAQTGWVQNQGILPGM |
| | | VWQDRDVYLQGPIWAKIPHTDGNFHPSPLMGGFGMKHPPPQILIKNTPVPADPPT |
| | | AFNKDKLNSFITQYSTGQVSVEIEWELQKENSKRWNPEIQYTSNYYKSNNVEFAV |
| | | NTEGVYSEPRPIGTRYLTRNL |
| 51 | AAV9 | MAADGYLPDWLEDNLSEGIREWWALKPGAPQPKANQQHQDNARGLVLPGYKYLGP |
| | | GNGLDKGEPVNAADAAALEHDKAYDQQLKAGDNPYLKYNHADAEFQERLKEDTSF |
| | | GGNLGRAVFQAKKRLLEPLGLVEEAAKTAPGKKRPVEQSPQEPDSSAGIGKSGAQ |
| | | PAKKRLNFGQTGDTESVPDPQPIGEPPAAPSGVGSLTMASGGGAPVADNNEGADG |
| | | VGSSSGNWHCDSQWLGDRVITTSTRTWALPTYNNHLYKQISNSTSGGSSNDNAYF |
| | | GYSTPWGYFDFNRFHCHFSPRDWQRLINNNWGFRPKRLNFKLFNIQVKEVTDNNG |
| | | VKTIANNLTSTVQVFTDSDYQLPYVLGSAHEGCLPPFPADVFMIPQYGYLTLNDG |
| | | SQAVGRSSFYCLEYFPSQMLRTGNNFQFSYEFENVPFHSSYAHSQSLDRLMNPLI |
| | | DQYLYYLSKTINGSGQNQQTLKESVAGPSNMAVQGRNYIPGPSYRQQRVSTTVTQ |
| | | NNNSEFAWPGASSWALNGRNSLMNPGPAMASHKEGEDRFFPLSGSLIFGKQGTGR |
| | | DNVDADKVMITNEEEIKTTNPVATESYGQVATNHQSAQAQAQTGWVQNQGILPGM |
| | | VWQDRDVYLQGPIWAKIPHTDGNFHPSPLMGGFGMKHPPPQILIKNTPVPADPPT |
| | | AFNKDKLNSFITQYSTGQVSVEIEWELQKENSKRWNPEIQYTSNYYKSNNVEFAV |
| | | NTEGVYSEPRPIGTRYLTRNL |
| 52 | Vascular | MNFLLSWVHWSLALLLYLHHAKWSQAAPMAEGGGQNHHEVVKFMDVYQRSYCHPI |
| | endothelial | ETLVDIFQEYPDEIEYIFKPSCVPLMRCGGCCNDEGLECVPTEESNITMQIMRIK |
| | growth factor | PHQGQHIGEMSFLQHNKCECRPKKDRARQENPCGPCSERRKHLFVQDPQTCKCSC |
| | (vegf) | KNTDSRCKARQLELNERTCRCDKPRR |
| | Caa44447.1 | |
| 53 | Palmitoyl- | MASPGCLWLLAVALLPWTCASRALQHLDPPAPLPLVIWHGMGDSCCNPLSMGAIK |
| | protein | KMVEKKIPGIYVLSLEIGKTLMEDVENSFFLNVNSQVTTVCQALAKDPKLQQGYN |
| | thioesterase 1 | AMGFSQGGQFLRAVAQRCPSPPMINLISVGGQHQGVFGLPRCPGESSHICDFIRK |
| | (ppt1) | TLNAGAYSKVVQERLVQAEYWHDPIKEDVYRNHSIFLADINQERGINESYKKNLM |
| | Aah08426.1 | ALKKFVMVKFLNDSIVDPVDSEWFGFYRSGQAKETIPLQETSLYTQDRLGLKEMD |
| | | NAGQLVFLATEGDHLQLSEEWFYAHIIPFLG |
| 54 | Tripeptidyl- | MGLQACLLGLFALILSGKCSYSPEPDQRRTLPPGWVSLGRADPEEELSLTFALRQ |
| | peptidase 1 | QNVERLSELVQAVSDPSSPQYGKYLTLENVADLVRPSPLTLHTVQKWLLAAGAQK |
| | (tpp1) | CHSVITQDFLTCWLSIRQAELLLPGAEFHHYVGGPTETHVVRSPHPYQLPQALAP |
| | Np_000382.3 | HVDFVGGLHRFPPTSSLRQRPEPQVTGTVGLHLGVTPSVIRKRYNLTSQDVGSGT |
| | | SNNSQACAQFLEQYFHDSDLAQFMRLFGGNFAHQASVARVVGQQGRGRAGIEASL |
| | | DVQYLMSAGANISTWVYSSPGRHEGQEPFLQWLMLLSNESALPHVHTVSYGDDED |
| | | SLSSAYIQRVNTELMKAAARGLTLLFASGDSGAGCWSVSGRHQFRPTFPASSPYV |
| | | TTVGGTSFQEPFLITNEIVDYISGGGFSNVFPRPSYQEEAVTKFLSSSPHLPPSS |
| | | YFNASGRAYPDVAALSDGYWVVSNRVPIPWVSGTSASTPVFGGILSLINEHRILS |
| | | GRPPLGFLNPRLYQQHGAGLFDVTRGCHESCLDEEVEGQGFCSGPGWDPVTGWGT |
| | | PNFPALLKTLLNP |
| 55 | Mutant | MYRMQLLLLIALSLALVINS |
| | interleukin-2 | |
| | signal peptide | |

4.5 Diseases

The pharmaceutical composition or the reference pharmaceutical composition provided herein (e.g., Section 4.1) can be administered to a subject diagnosed with nAMD (wet AMD), dry AMD, retinal vein occlusion (RVO), diabetic macular edema (DME), diabetic retinopathy (DR), or Batten disease.

In some embodiments, disclosed herein are methods of treating a subject diagnosed with nAMD (wet AMD), dry AMD, retinal vein occlusion (RVO), diabetic macular edema (DME), diabetic retinopathy (DR), or Batten by administering to the subject a therapeutically effective amount of the pharmaceutical composition by suprachoroidal injection (for example, via a suprachoroidal drug delivery device such as a microinjector with a microneedle).

In some embodiments, a pharmaceutical composition containing about 2.5×10¹¹ GC/eye, about 5×10¹¹ GC/eye, or about 1.5×10¹² GC/eye of Construct II of a pharmaceutical composition comprising 0.2 mg/mL potassium chloride, 0.2 mg/mL potassium phosphate monobasic, 5.84 mg/mL sodium chloride, 1.15 mg/mL sodium phosphate dibasic anyhydrous, 40.0 mg/mL, 4% w/v sucrose, and optionally a surfactant is administered to a patient via suprachoroidal administration. In some embodiments, the patient has diabetic retinopathy.

In some embodiments, a pharmaceutical composition containing about 2.5×10¹¹ GC/eye, about 5×10¹¹ GC/eye, or about 1.5×10¹² GC/eye of Construct II of a pharmaceutical composition comprising 10% w/v sucrose is administered to a patient via suprachoroidal administration. In some embodiments, the patient has diabetic retinopathy. In some embodiments, the pharmaceutical composition has a tonicity/osmolality equal to or greater than 240 mOsm/kg.

In some aspects, disclosed herein are pharmaceutical compositions suitable for, or methods of, treating a subject diagnosed with mucopolysaccharidosis type IVA (MPS IVA), mucopolysaccharidosis type I (MPS I), mucopolysaccharidosis type II (MPS II), familial hypercholesterolemia (FH), homozygous familial hypercholesterolemia (HoFH), coronary artery disease, cerebrovascular disease, Duchenne muscular dystrophy, Limb Girdle muscular dystrophy, Becker muscular dystrophy and sporadic inclusion body myositis, or kallikrein-related disease comprising administering to the subject a therapeutically effective amount of the pharmaceutical composition. In some embodiments, the pharmaceutical composition is administered in the SCS.

In some embodiments, the pharmaceutical composition or the reference pharmaceutical composition provided herein (e.g., Section 4.1) can be administered to a subject diagnosed with (1) Batten-CLN2 and the therapeutic product is Tripeptidyl-Peptidase 1 (TPP1); (2) Usher's-Type 1 and the therapeutic product is Myosin VIIA (MYO7A); (3) Usher's-Type 1 and the therapeutic product is Cadherin Related 23 (CDH23); (4) Usher's-Type 2 and the therapeutic product is Protocadherin Related 15 (PCDH15); (5) Usher's-Type 2 and the therapeutic product is Usherin (USH2A); (6) Usher's-Type 3 and the therapeutic product is Clarin 1 (CLRN1); (7) Stargardt's and the therapeutic product is ATP Binding Cassette Subfamily A Member 4 (ABCA4); (8) Stargardt's and the therapeutic product is ELOVL Fatty Acid Elongase 4 (ELOVL4); (9) red-green color blindness and the therapeutic product is L opsin (OPN1LW); (10) red-green color blindness and the therapeutic product is M opsin (OPN1MW); (11) blue cone monochromacy and the therapeutic product is M opsin (OPN1MW); (12) Leber congenital amaurosis-1 (LCA 1) and the therapeutic product is Guanylate Cyclase 2D, Retinal (GUCY2D); (13) Leber congenital amaurosis-2 (LCA 2) and the therapeutic product is Retinoid Isomerohydrolase RPE65 (RPE65); (14) Leber congenital amaurosis-4 (LCA 4) and the therapeutic product is Aryl Hydrocarbon Receptor Interacting Protein Like 1 (AIPL1); (15) Leber congenital amaurosis-7 (LCA 7) and the therapeutic product is Cone-Rod Homeobox (CRX); (16) Leber congenital amaurosis-8 (LCA 8) and the therapeutic product is Crumbs Cell Polarity Complex Component 1 (CRB1); (17) Leber congenital amaurosis-9 (LCA 9) and the therapeutic product is Nicotinamide Nucleotide Adenylyltransferase 1 (NMNAT1); (18) Leber congenital amaurosis-10 (LCA 10) and the therapeutic product is Centrosomal Protein 290 (CEP290); (19) Leber congenital amaurosis-11 (LCA 11) and the therapeutic product is Inosine Monophosphate Dehydrogenase 1 (IMPDH1); (20) Leber congenital amaurosis-15 (LCA 15) and the therapeutic product is Tubby Like Protein 1 (TULP1); (21) LHON and the therapeutic product is Mitochondrially Encoded NADH Dehydrogenase 4 (MT-ND4); (22) LHON and the therapeutic product is Mitochondrially Encoded NADH Dehydrogenase 6 (MT-ND6); (23) choroideremia and the therapeutic product is Rab Escort Protein 1 (CHM); (24) X-linked retinoschisis (XLRS) and the therapeutic product is Retinoschisin (RS1); (25) Bardet-Biedl syndrome 1 and the therapeutic product is Bardet-Biedl Syndrome 1 (BB S1); (26) Bardet-Biedl syndrome 6 and the therapeutic product is McKusick-Kaufman Syndrome (MKKS); (27) Bardet-Biedl syndrome 10 and the therapeutic product is Bardet-Biedl Syndrome 10 (BB S10); (28) cone dystrophy and the therapeutic product is Guanylate Cyclase Activator 1A (GUCA1A); (29) optic atrophy and the therapeutic product is OPAL Mitochondrial Dynamin Like GTPase (OPAL); (30) retinitis pigmentosa 1 and the therapeutic product is RP1 Axonemal Microtubule Associated (RP1); (31) retinitis pigmentosa 2 and the therapeutic product is RP2 Activator of ARL3 GTPase (RP2); (32) retinitis pigmentosa 7 and the therapeutic product is Peripherin 2 (PRPH2); (33) retinitis pigmentosa 11 and the therapeutic product is Pre-mRNA Processing Factor 31(PRPF31); (34) retinitis pigmentosa 13 and the therapeutic product is Pre-mRNA Processing Factor 8 (PRPF8); (35) retinitis pigmentosa 37 and the therapeutic product is Nuclear Receptor Subfamily 2 Group E Member 3 (NR2E3); (36) retinitis pigmentosa 38 and the therapeutic product is MER Proto-Oncogene, Tyrosine Kinase (MERTK); (37) retinitis pigmentosa 40 and the therapeutic product is Phosphodiesterase 6B (PDE6B); (38) retinitis pigmentosa 41 and the therapeutic product is Prominin 1 (PROM1); (39) retinitis pigmentosa 56 and the therapeutic product is Interphotoreceptor Matrix Proteoglycan 2 (IMPG2); (40) petinitis pigmentosa 62 and the therapeutic product is Male Germ Cell Associated Kinase (MAK); (41) retinitis pigmentosa 80 and the therapeutic product is Intraflagellar Transport 140 (IFT140); or (42) Best disease and the therapeutic product is Bestrophin 1 (BEST1).

4.6 Assays

The skilled artesian may use the assays as described herein and/or techniques known in the art to study the composition and methods described herein, for example to test the formulations provided herein. As detailed in Section 5, the following assays are also provided herein.

4.6.1 Ultrasound B-Scan

A high-frequency ultrasound (U/S) probe (LBM Plus; Accutome, Malvern, PA, USA) can be used to determine SCS thickness by generating 2D cross-sectional images of the SCS in animal eyes ex vivo after injecting different volumes ranging in viscosity (e.g., from 25 μL to 500 μL ranging from low viscosity to high viscosity). An U/S probe cover (Clearscan, Eye-Surgical-Instruments, Plymouth, MN) can be attached to the UBM Plus to facilitate U/S image acquisition. The U/S probe can be used to acquire sagittal views around the eye (e.g., eight sagittal views). Postprocessing of the U/S B-scans can be performed to find the thickness from the outer sclera to the inner retina at, for example, 1, 5, and 9 mm posterior to the scleral spur. The mean, median, and standard deviation for each eye can be calculated.

4.6.2 Measuring SCS Thickness Based on Liquid Volume

3D cryo-reconstruction imaging can be used to measure SCS thickness. Animal eyes that are injected with, for example, 25 μL to 500 μL containing red-fluorescent particles are frozen a few minutes (e.g., 3-5 minutes) post injection and prepared for cryosectioning. Using a digital camera, one red-fluorescent image of the cryoblock of tissue can be obtained every 300 μm by slicing the sample with the cryostat. Image stacks consisting of red-fluorescence images are analyzed to determine SCS thickness.

4.6.3 Measuring SCS Thickness Based on Formulation

U/S B-scan can be used to determine SCS thickness after injection of pharmaceutical compositions ranging in viscosity into the SCS of animals. High-frequency ultrasound B-scan can be used to determine the rate of SCS collapse. Eight sagittal views over the pars plana can be acquired: (a) supranasal, over the injection site; (b) superior; (c) nasal; (d) supratemporal; (e) temporal; (f) infratemporal; (g) inferior; and (h) infranasal.

Off-line post processing can be performed on the U/S views to measure the SCS thickness. The U/S probe can have a minimum axial resolution of 15 μm. For each U/S view, a line segment 5 mm posterior to the scleral spur and perpendicular to the sclera can be created. A line can start at the outer surface of the sclera and end at the inner surface of the retina. The sclera and chorioretina can be included in the measurement to ensure the line is perpendicular. SCS thickness is then calculated by subtracting the tissue thickness from the measured line length. Curve fitting is done to determine the rate of SCS collapse.

U/S B-scan can be used to determine SCS thickness at multiple locations over time and the rate of SCS collapse can be calculated. The approximate clearance rate of injected fluorescent material from the SCS can be found by taking fluorescence fundus images in the animal eyes in vivo over time until fluorescence is no longer detected.

4.6.4 SCS Clearance Kinetics by Fundus Imaging

To study the effect of AAV aggregation on movement in the SCS, different pharmaceutical compositions ranging in AAV aggregation levels and containing a fluorescein can be injected into the SCS. The approximate clearance rate or clearance time of injected fluorescent material from the SCS can be found by taking fluorescence fundus images over time in animal eyes in vivo. In some cases, the rate of clearance can be determined by determining the total clearance time and the clearance time constant (t_{clearance}) calculated using a curve fit derived from the normalized concentration of total fluorescent signal over time. Topical eye drops of tropicamide and phenylephrine (Akorn, Lake Forest, IL) can be administered prior to each imaging session to dilate the eye. A RetCam II (Clarity Medical Systems, Pleasanton, CA) with the 130° lens attachment and the built-in fluorescein angiography module can be used to acquire the images. Multiple images can be taken with the blue light output from the RetCam II set at, for example, 0.0009, 1.6, and 2.4 W/m². In an attempt to capture the entire interior surface of the ocular globe, nine images can be captured: central, supranasal, superior, supratemporal, temporal, infratemporal, inferior, infranasal, and nasal. This allows imaging into the far periphery. Imaging can be done immediately after injection, at 1 h, every 3 h for 12 h, and every two days post-injection. The total clearance time, which can be defined as the first time point following injection in which fluorescence is not detectable by visual observation, is determined for all eyes injected. Fluorescein isothiocyanate-conjugated AAV (FITC-AAV), or FITC Conjugated-AAV capsid Protein-specific monoclonal antibody may be utilized in analogous experiments to track movement and clearance of AAV particles in the SCS. Methods for fluorescent labeling of AAV are known in the art (Shi, et al. Sci. Adv. 2020; 6: eaaz3621; and Tsui, T. Y., et al. Hepatology 42, 335-342 (2005). Antibodies (FITC Conjugated) recognizing many AAV serotypes are commercially available.

4.6.5 Flat Mount to Characterize 2D Circumferential Spread

Pharmaceutical compositions of the present disclosure containing fluorescein, or fluorescently labeled AAV, are injected into the SCS. After SCS injection and freezing, eyes can be prepared to assess the 2D spread of particles and fluorescein. The frozen eye are sliced open from the limbus to the posterior pole to generate equidistant scleral flaps. The resulting scleral flaps are splayed open and the frozen vitreous humor, lens, and aqueous humor are removed.

A digital SLR camera (Canon 60D, Canon, Melville, N.Y.) with a 100 mm lens (Canon) can be used to acquire brightfield and fluorescence images. Camera parameters are held constant. To acquire the area of fluorescein spread, a green optical band-pass filter (520±10 nm; Edmunds Optics, Barrington, N.J.) can be placed on the lens, and the sample can be illuminated by a lamp with the violet setting of a multicolor LED bulb (S Series RGB MR16/E26. HitLights, Baton Rouge, La.). To visualize the location of the red-fluorescent particles, a red filter (610±10 nm; Edmunds Optics) can be placed on the lens, and the sample can be illuminated with the same lamp switched to green light. The area of green and red fluorescence that are above threshold can be calculated for each eye using ImageJ (National Institutes of Health, Bethesda, Md.). Thresholding can be set manually based on visual inspection of background signal.

4.6.6 Intraocular Pressure Measurements

A pressure measurement system can be used to measure pressure in SCS after SCS injection. Animals can be terminally anesthetized by subcutaneous injection of a ketamine/xylazine cocktail. After SCS injection (N=4), pressure in the SCS can be measured every few minutes. Pressures are monitored until they reach their original baseline values from before injection (i.e., ˜15 mmHg). After the measurements, the animals are euthanized with a lethal dose of pentobarbital injected intravenously. A second set of SCS injections can be made in the animal postmortem. In postmortem measurements, pressure is only measured in the tissue space (i.e., SCS) where the injection was made.

4.6.7 Temperature Stress Assay

A temperature stress development stability study can be conducted at 1.0×10¹² GC/mL over 4 days at 37° C. to evaluate the relative stability of formulations provided herein. Assays can be used to assess stability include but are not limited to in vitro relative potency (IVRP), vector genome concentration (VGC by ddPCR), free DNA by dye fluorescence, dynamic light scattering, appearance, and pH. Long-term development stability studies can be carried out for 12 months to demonstrate maintenance of in-vitro relative potency and other quality at −80° C. (≤−60° C.) and −20° C. (−25° C. to −15° C.) in the formulations provided herein.

4.6.8 In Vitro Relative Potency (IVRP) Assay

To relate the ddPCR GC titer to gene expression, an in vitro bioassay may be performed by transducing HEK293 cells and assaying the cell culture supernatant for anti-VEGF Fab protein levels. HEK293 cells are plated onto three poly-D-lysine-coated 96-well tissue culture plates overnight. The cells are then pre-infected with wild-type human Ad5 virus followed by transduction with three independently prepared serial dilutions of AAV vector reference standard and test article, with each preparation plated onto separate plates at different positions. On the third day following transduction, the cell culture media is collected from the plates and measured for VEGF-binding Fab protein levels via ELISA. For the ELISA, 96-well ELISA plates coated with VEGF are blocked and then incubated with the collected cell culture media to capture anti-VEGF Fab produced by HEK293 cells. Fab-specific anti-human IgG antibody is used to detect the VEGF-captured Fab protein. After washing, horseradish peroxidase (HRP) substrate solution is added, allowed to develop, stopped with stop buffer, and the plates are read in a plate reader. The absorbance or OD of the HRP product is plotted versus log dilution, and the relative potency of each test article is calculated relative to the reference standard on the same plate fitted with a four-parameter logistic regression model after passing the parallelism similarity test, using the formula: EC50 reference÷EC50 test article. The potency of the test article is reported as a percentage of the reference standard potency, calculated from the weighted average of the three plates.

To relate the ddPCR GC titer to functional gene expression, an in vitro bioassay may be performed by transducing HEK293 cells and assaying for transgene (e.g. enzyme) activity. HEK293 cells are plated onto three 96-well tissue culture plates overnight. The cells are then pre-infected with wild-type human adenovirus serotype 5 virus followed by transduction with three independently prepared serial dilutions of enzyme reference standard and test article, with each preparation plated onto separate plates at different positions. On the second day following transduction, the cells are lysed, treated with low pH to activate the enzyme, and assayed for enzyme activity using a peptide substrate that yields increased fluorescence signal upon cleavage by transgene (enzyme). The fluorescence or RFU is plotted versus log dilution, and the relative potency of each test article is calculated relative to the reference standard on the same plate fitted with a four-parameter logistic regression model after passing the parallelism similarity test, using the formula: EC50 reference÷EC50 test article. The potency of the test article is reported as a percentage of the reference standard potency, calculated from the weighted average of the three plates.

4.6.9 Vector Genome Concentration Assay

Vector genome concentration (GC) can also be evaluated using ddPCR. At various timepoints post injection, several mice are sacrificed, and ocular tissues are subjected to total DNA extraction and ddPCR assay for vector copy numbers. Copies of vector genome (transgene) per gram of tissue identified in various tissue sections at sequential timepoints will reveal spread of AAV in the eye.

Total DNA from collected ocular tissue sections are extracted with the DNeasy Blood & Tissue Kit and the DNA concentration re measured using a Nanodrop spectrophotometer. To determine the vector copy numbers in the tissue sections, digital PCR was performed with Naica Crystal Digital PCR system (Stilla technologies). Two color multiplexing system were applied here to simultaneously measure the transgene AAV and an endogenous control gene. In brief, the transgene probe can be labelled with FAM (6-carboxyfluorescein) dye while the endogenous control probe can be labelled with VIC fluorescent dye. The copy number of delivered vector in a specific tissue section per diploid cell is calculated as: (vector copy number)/(endogenous control)×2. Vector copy in specific cell types, such as RPE cells may reveal sustained delivery to the retina.

4.6.10 Free DNA Analysis Using Dye Fluorescence Assay

Free DNA can be determined by fluorescence of SYBR® Gold nucleic acid gel stain (‘SYBR Gold dye’) that is bound to DNA. The fluorescence can be measured using a microplate reader and quantitated with a DNA standard. The results in ng/μL can be reported.

Two approaches can be used to estimate the total DNA in order to convert the measured free DNA in ng/μL to a percentage of free DNA. In the first approach the GC/mL (OD) determined by UV-visible spectroscopy was used to estimate the total DNA in the sample, where M is the molecular weight of the DNA and 1E6 is a unit conversion factor:

TotalDNA(ng/μL)estimated=1E6×GC/mL(OD)×M(g/mol)/6.02E23

In the second approach, the sample can be heated to 85° C. for 20 min with 0.05% poloxamer 188 and the actual DNA measured in the heated sample by the SYBR Gold dye assay can be used as the total. This therefore has the assumption that all the DNA was recovered and quantitated. For trending, either the raw ng/μL can be used or the percentage determined by a consistent method can be used.

4.6.11 Size Exclusion Chromatography (SEC)

SEC can be performed using a Sepax SRT SEC-1000 Peek column (PN 215950P-4630, SN: 8A11982, LN: BT090, 5 μm 1000A, 4.6×300 mm) on Waters Acquity Arc Equipment ID 0447 (C3PO), with a 25 mm pathlength flowcell. The mobile phase can be, for example, 20 mM sodium phosphate, 300 mM NaCl, 0.005% poloxamer 188, pH 6.5, with a flow rate of 0.35 mL/minute for 20 minutes, with the column at ambient temperature. Data collection can be performed with 2 point/second sampling rate and 1.2 nm resolution with 25 point mean smoothing at 214, 260, and 280 nm. The ideal target load can be 1.5E11 GC. The samples can be injected with 50 μL, about ⅓ of the ideal target or injected with 5 μL.

4.6.12 Dynamic Light Scattering (DLS) Assay

Dynamic light scattering (DLS) can be performed on a Wyatt DynaProIII using Corning 3540 384 well plates with a 30 μL sample volume. Ten acquisitions each for 10 s can be collected per replicate and there can be three replicate measurements per sample. The solvent can be set according to the solvent used in the samples, for example ‘PBS’ for an AAV vector in dPBS. Results not meeting data quality criteria (baseline, SOS, noise, fit) can be ‘marked’ and excluded from the analysis.

4.6.13 Viscosity Measurement

Viscosity can be measured using methods known in the art, for example methods provide in the United States Pharmacopeia (USP) published in 2019 and previous versions thereof (incorporated by reference herein in their entirety). Viscosity at low shear was measured using a capillary viscometer, using methods described in USP <911>.

Viscosity versus shear rate was determined using a cone and plate rotational rheometer. Rheometry measurements are described in the United States Pharmacopeia (USP) USP <1911> and rotational viscometry is described in USP<912>. Rotational rheometry viscosity measurements were collected with an AR-G2 rheometer equipped with a Peltier temperature control plate with a 60 mm 1° angle aluminum cone accessory (TA Instruments, New Castle, DE). A viscosity versus shear rate sweep was performed over the range starting at <0.3 s⁻¹ ramped up to 5000 s⁻¹ with 5 points per decade collected. The viscosity versus shear rate was collected at 20° C. Viscosity at 10,000 and 20,000 s⁻¹ were extrapolated from the data. In some cases, the viscosity of the pharmaceutical composition or the reference pharmaceutical composition can be measured at zero, 0.1 s⁻¹, 1 s⁻¹, 1000 s⁻¹, 5000 s⁻¹, 10,000 s⁻¹, 20,000 s⁻¹, or more than 20,000 s⁻¹.

4.6.14 Virus Infectivity Assay

TCID₅₀ infectious titer assay as described in Francois, et al. Molecular Therapy Methods & Clinical Development (2018) Vol. 10, pp. 223-236 (incorporated by reference herein in its entirety) can be used. Relative infectivity assay as described in Provisional Application 62/745,859 filed Oct. 15, 2018) can be used

4.6.15 Differential Scanning Fluorimetry

The thermal stability of proteins and virus capsids made up of proteins can be determined by differential scanning fluorimetry (DSF). DSF measures the intrinsic tryptophan and tyrosine emission of proteins as a function of temperature. The local environment of Trp and Tyr residues changes as the protein unfolds resulting in a large increase in fluorescence. The temperature where 50% of proteins are unfolded is defined as the ‘melting’ temperature (Tₘ). Fluorescence spectroscopy is described in the USP <853> and USP <1853>.

DSF data was collected using a Promethius NTPlex Nano DSF Instrument (NanoTemper technologies, Munich, Germany). Samples were loaded into the capillary cell at 20° C. and the temperature ramped at a rate of 1° C./min to 95° C. The signal output ratio of emission at 350 nm (unfolded) and 330 nm (unfolded) was used to determine the Tₘ

4.6.16 Injection Pressure Measurements

Injection pressures were measured using either a Flow Screen and Fluid Sensor (Viscotec America, Kennesaw, GA) or a PressureMAT-DPG with single use pressure sensor S-N-000 (PendoTECH, Princeton, NJ).

Injections into air were either performed manually or using a Legato-100 syringe pump (Kd Scientific, Holliston, MA) to apply a consistent flow rate. For injections into enucleated porcine eyes, the eyes were mounted on a Mandell eye mount (Mastel) with applied suction to adjust the introcular pressure of the eye.

4.6.17 Reference Compositions

The viscosity of a composition provided herein may be evaluated by comparing the composition to a reference pharmaceutical composition. In some embodiments, the reference pharmaceutical composition is a pharmaceutical composition comprising the same recombinant AAV in the same concentration as the composition being evaluated in phosphate-buffered saline. In some embodiments, the reference pharmaceutical composition is a pharmaceutical composition comprising the same recombinant AAV in the same concentration as the composition being evaluated in Dulbecco's phosphate buffered saline with 0.001% poloxamer 188, pH 7.4. In some embodiments, the reference pharmaceutical composition is a pharmaceutical composition comprising the same recombinant AAV in the same concentration as the composition being evaluated in Dulbecco's phosphate buffered saline with 4% sucrose and poloxamer 188, pH 7.4.

### EXAMPLES

The examples in this section (i.e., section 5) are offered by way of illustration, and not by way of limitation.

### Example 1: Optimization of Viscosity Formulation for Suprachoroidal Delivery

In this experiment, solutions containing AAV8-antiVEGF-ab (e.g., AAV8-antiVEGFfab) were evaluated for administration to the suprachoroidal space. The suprachoroidal space (SCS) is a region between the sclera and the choroid that expands upon injection of a drug solution (Habot-Wilner, 2019). The SCS space recovers to its pre-injection size as the injected solution is cleared by physiologic processes. The drug solution diffuses within the SCS and is absorbed into adjacent tissues. Capillaries in the choroid are permeable to low molecular weight osmolytes. Different solutions having different viscosity levels were injected in the suprachoroidal space to evaluate efficacy based on the solutions' residence time in the SCS.

In this experiment, longer residence time was achieved by formulating AAV in a final formulation that has high viscosity (shear-thinning with a lower viscosity when injected). There was an increase in localization and an increase in the clearance time when the AAV was injected in the SCS using a high viscosity formulation (with shear-thinning) as compared to lower viscosity formulations. This increase in localization and clearance time is indicative of enhanced efficacy. The shear-thinning behavior resulting in lower viscosity while being injected allows for a lower injection pressure compatible with a needle size (e.g., 29 or 30 gauge needle), standard polymeric syringes pressure rating limitations, and a low enough pressure that is appropriate for injection by physicians in human eyes.

5.1.1 Overview of Initial Design Parameters:

Delivery of a viscous formulation to the suprachoroidal space has design parameters and limitations that other routes of administration do not. Injection to the eye is a sensitive injection procedure as the eye is a critical organ. The needle gauge cannot be too high when injecting a drug in the eye in order to avoid pain, tissue damage, or inflammation. For example, in some cases, a 30 gauge can be selected and in other cases, a 29 gauge is selected. In contrast, 18 or 21 gauge needles may be used for routine injection into peripheral veins, and subcutaneous administration may use 29 to 27 gauge needles. Tissue damage or temporary inflammation in these areas is less critical than in the eye. Injection of viscous formulations by other routes of administration is therefore less restricted by needle gauge.

The pressure of injection is inversely proportional to the 4th power of needle inner diameter (ID) and is proportional to the formulation viscosity as shown in the Hagen-Poiseuille equation. The Hagen-Poiseuille equation is represented by ΔP=(8 μLQ)/(πR⁴). The pressure depends upon the viscosity (μ), the needle length (L), the volumetric flow rate (Q), and the inner radius of the needle (R). Therefore, needle gauge is critically limiting for suprachoroidal injection. Without other considerations, the logical approach would be to minimize formulation viscosity for delivery by injection to the suprachoroidal space. However, in opposition to this aspect, is the desire of the present disclosure to have a very high viscosity formulation in order to localize the delivered dose to the suprachoroidal space for a longer period of time. The fact that these factors are in direct opposition lead to the specific need for a shear-thinning formulation for suprachoroidal delivery while minimizing potential inflammation caused by injection.

In this experiment, different formulations that have acceptable viscosity and that are suitable for injection using commonly or reasonably available syringe components (i.e. an injection pressure limit based on syringe pressure-rating limitations, or needle gauge preference) were tested for suprachoroidal injection. There are conflicting design parameters to balance between low viscosity for injection and high viscosity for localization with the needle gauge limitation applied. In some cases, the desired needle size can be 30 or 29 gauge size. The gauge size impacts the pressure during injection. In some cases, the injection pressure corresponds with the industry standard components and specifications (e.g., ISO) for the components. Table 2 summarizes the design parameters.

TABLE 2
Design Parameters

| Parameter | Target | Rationale |
|---|---|---|
| Excipients safety | Excipients are safe and have | Safety |
| | been used previously for | |
| | parenterals and ideally for | |
| | delivery to the eye | |
| Stability of Vector | Vector is stable and retains | Stability is required to ensure efficacy |
| | potency | |
| Injection Time | Preferred (in some cases): 10 s | Clinical administration targets |
| | Range: 5 s to 30 s | |
| Administration | Preferred (in some cases): | Clinical administration requirement and |
| components | CLSD 30 Ga (160 μm ID) | ability to use standard components |
| | needle | |
| | Range: 29 or 30 Ga ETW | |
| | needle (ID = 220 or ID = | |
| | 240 μm) or a CLSD 30 Ga ETW | |
| | (220 μm ID) needle and | |
| | standard plastic syringe | |
| Injection Pressure | Preferred (in some cases) ≤43 | Preferred value is based on ISO 7886-1: |
| | PSI | 2017 for syringe pressure ratings, and |
| | Range ≤65 PSI | low/easy injection pressure and force. |
| | Limit ≤100 PSI | The range of 65 PSI is based on the |
| | | human factor/feeling of force needed |
| | | during laboratory injection feasibility |
| | | experiments |
| Viscosity at | Preferred (in some cases): 34 | Preferred (in some cases): injection |
| injection shear rate | mPas at room temperature | pressure target of up to 43 PSI when |
| | accounting for shear-thinning | using CLSD 30 Ga (160 μm ID) needle |
| | at injection shear rate | and 10 s injection. |
| | Range: 103 mPas, 121 mPas | Range: injection pressure target using a |
| | and up to 362 mPas (cP) at | CLSD 30 Ga ETW (220 μm ID) needle |
| | room temp accounting for | for injection with a pressure target of up |
| | shear-thinning at injection | to 43 PSI and 10 s injection. |
| | shear rate | Alternatively, an injection pressure target |
| | | of up to 65 PSI when using CLSD 30 Ga |
| | | (160 μm ID) needle or a CLSD 30 Ga |
| | | ETW (220 μm ID) needle, or an injection |
| | | pressure target of 43 PSI for a 30 s |
| | | injection is used. |
| Viscosity at low | Preferred (in some cases): ≥10 | higher viscosity at low shear will result in |
| shear (1 s⁻¹) | times the viscosity at high | greater localization of the dose |
| | shear or maximize | |
| | Range: ≥5 times the viscosity | |
| | at high shear | |

### 5.2 Example 2: Distribution of a Solution in the Suprachoroidal Space (SCS) is Dependent on the Viscosity of the Solution

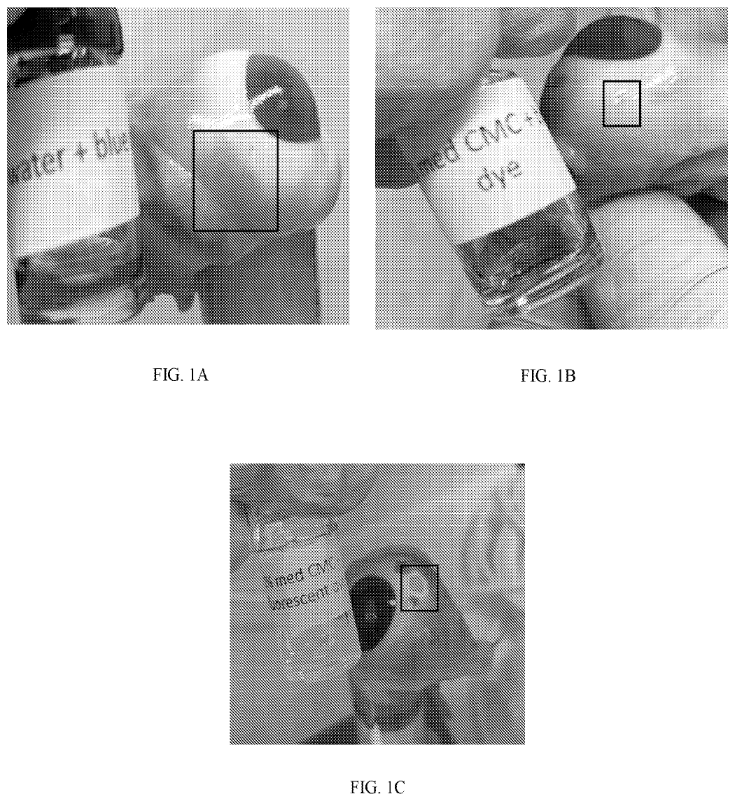

In this experiment, solutions having different viscosities were injected in eyes ex vivo to analyze the impact of fluid viscosity on localization and spreading of the solutions in the SCS. Three different solutions were injected in different eyes ex vivo: 1) a solution containing water and blue dye; 2) a solution containing 1% carboxymethyl cellulose (CMC) medium viscosity grade and blue dye; and 3) a solution containing 1% CMC medium and fluorescent dye. The CMC solutions were prepared in a solution also containing 0.2 mg/mL potassium chloride, 0.2 mg/mL potassium phosphate monobasic, 5.84 mg/mL sodium chloride, 1.15 mg/mL sodium phosphate dibasic anyhydrous, 40.0 mg/mL (4% w/v) sucrose, and 0.001% (0.01 mg/mL) poloxamer 188, pH 7.4. The eyes were visually analyzed at different time points after injection. This experiment showed that fluid viscosity had a significant impact on the spreading of the solution in the SCS. For example, the solution containing water and blue dye resulted in a larger circumferential spread as compared to the solutions containing 1% CMC (FIGS. 1A-1C). The blue dye and fluorescent dye were significantly more localized in the eyes (in the SCS) when the eyes were injected with a solution containing 1% CMC as compared to a solution containing water (FIGS. 1A-1C). This experiment showed that an agent of interest (e.g., AAV, drug, or composition) can be localized in the SCS with minimal spreading when a solution having a desired fluid viscosity is injected in the SCS.

### 5.3 Example 3: Effect of Viscosity, Injection Rate, and Needle Gauge on Injection Pressure

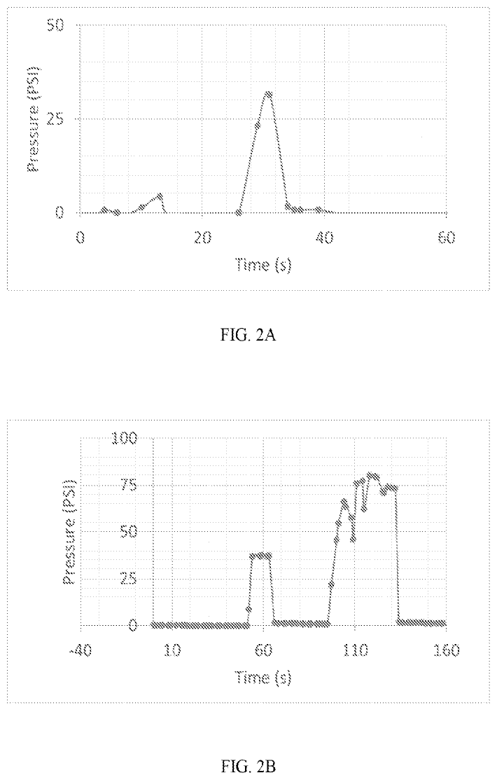

In this experiment, different solutions having different viscosities were injected in the SCS of several eyes. The hypromellose solutions were prepared in a ‘base’ solution also containing 0.2 mg/mL potassium chloride, 0.2 mg/mL potassium phosphate monobasic, 5.84 mg/mL sodium chloride, 1.15 mg/mL sodium phosphate dibasic anyhydrous, 40.0 mg/mL (4% w/v) sucrose, and 0.001% (0.01 mg/mL) poloxamer 188, pH 7.4. The extra excipients were dissolved in this solution. In short, a solution containing water and a solution containing 2% hydroxypropyl methylcellulose (hypromellose) were injected separately and in different eyes. Injection in the SCS can be performed by using, for example, the SCS Microinjector™ (Clearside Biomedical, Alpharetta, GA). This experiment showed that the amount of pressure required to inject a solution in the SCS was impacted by the viscosity of the solution. For example, the amount of pressure required to inject a water solution (viscosity of about 1 cP) in the SCS was found to be about 25 PSI while the amount of pressure required to inject a solution containing 2% hypromellose (viscosity of about 4000 cP) ranged from about 25 PSI to about 40 PSI (FIGS. 2A-2B). This pressure is much lower than would be predicted based on behavior of a Newtonian (constant viscosity versus shear rate) fluid. This experiment also showed that shear thinning is preferred for very viscous solutions. For shear-thinning fluids, the viscosity is very high at low shear (static or very low flow rate for example) and decreases as the shear rate increases or when the flow rate increases. A lower viscosity at the shear rate during injection reduces the pressure required to inject the dose through the needle to ranges compatible with the desired needle and syringes. After injection, the viscosity of the fluid is much higher (since the fluid is no longer in movement), thus reducing the spread of the solutions and keeping the injected dose localized for a longer period of time.

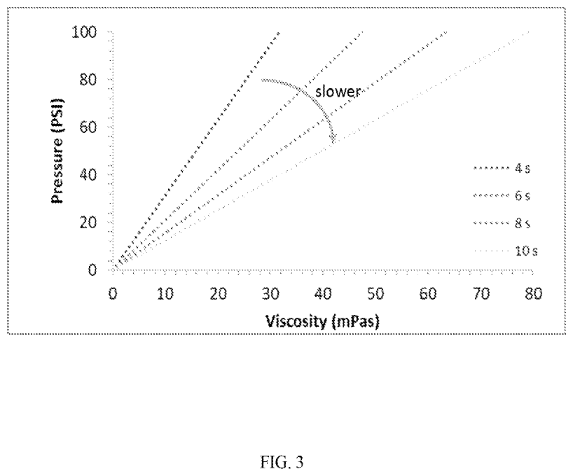

This design-space study also analyzed the impact of injection rates on the calculated amount of pressure required to inject different solutions in the SCS. For this design-space feasibility calculation, injection rates of 4 seconds, 6 seconds, 8 seconds, or 10 seconds were calculated for solutions of different viscosities in the SCS via a 30 gauge (GA) needle with a 160 μm inner diameter (ID). The Hagen-Poiseuille equation for pressure drop during flow through a needle is given by ΔP=(8 μLQ)/(πR⁴). The pressure depends upon the viscosity (μ), the needle length (L), the volumetric flow rate (Q), and the inner radius of the needle (R). The slowest injection rate (i.e., 10 seconds) resulted in the least amount of calculated pressure (FIG. 3). For example, the data in FIG. 3 shows that a solution with a viscosity of 10 mPas injected at a rate of 10 seconds results in a pressure of about 12 PSI but about 28 PSI when injected at a rate of 4 seconds. This calculation showed that the pressure is even more impacted by the rate of injection as the viscosity of the solution increased (FIG. 3). For example, a solution with a viscosity of 30 mPas is calculated to result in a pressure of about 36 PSI during a 10 seconds rate of injection and about 96 PSI during a 4 seconds rate of injection (FIG. 3). In some cases, the injection time ranges between 5 s to 30 s. In some cases, an injection time between 10 s to 15 s is used to inject solutions via suprachoroidal administration. The viscosity of a shear-thinning fluid depends on the shear rate (γ̊). Therefore, the predicted injection pressure depends upon the injection speed in terms of the flow rate directly and also on the shear-thinning viscosity behavior of the fluid. The average shear rate of the fluid flowing through the needle can be calculated using <γ̊>=(8Q)/(3πR³) (Jaspe, J.; Hagen, S. J. Biophys J 2006, 91, 3415-3424). For the example shown in FIG. 3, the shear rate ranges from about 16,500 s⁻¹ for a 10 s injection to 41,000 s⁻¹ for a 4 s injection. For a 20 s injection the shear rate is about 8000 s⁻¹ and is about 5000 s⁻¹ for a 30 s injection.

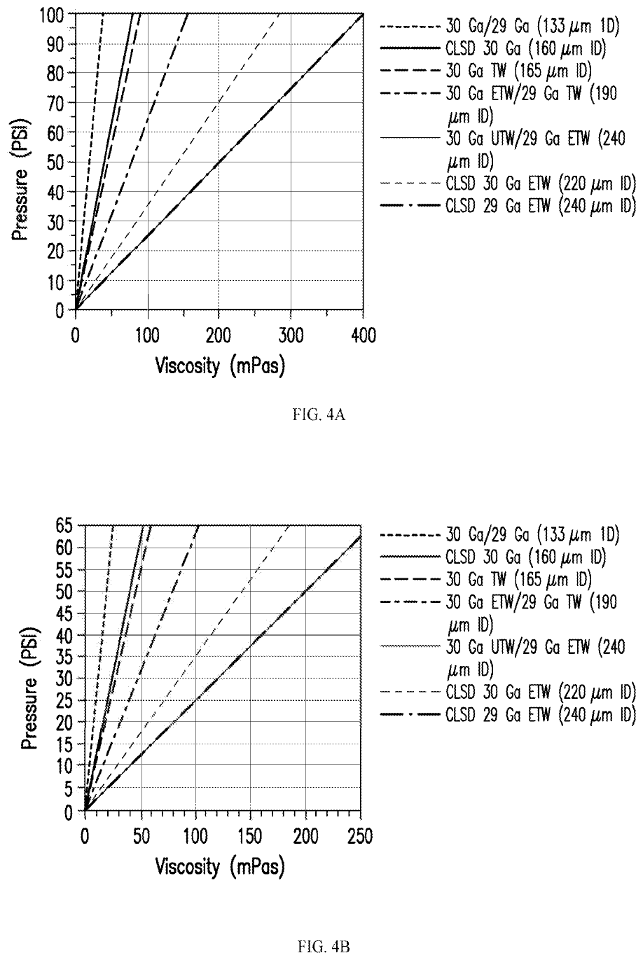

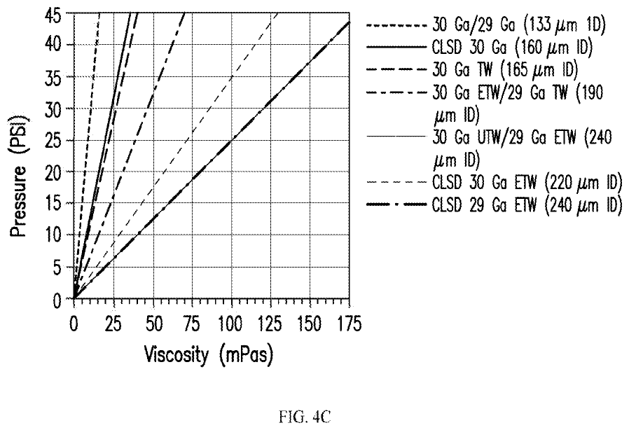

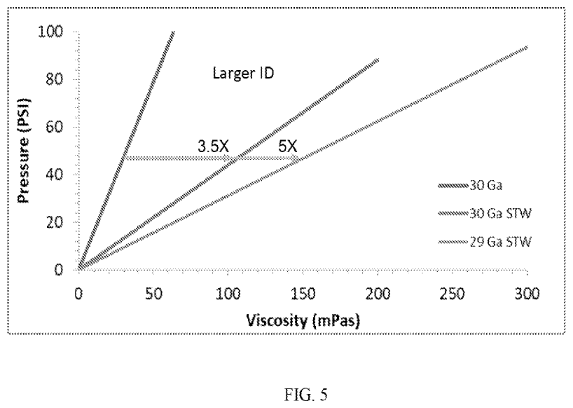

Using the Hagen-Poiseuille equation and the equation for average shear rate, the preferred formulation viscosity characteristics and the range for a certain pressure limitation during injection can be calculated. The change in pressure can be evaluated based on the needle gauge (FIGS. 4A-4C and FIG. 5). Three different sizes of needle gauges were used (e.g., a 30 gauge needle, a 30 gauge STW needle, and a 29 gauge STW needle) to inject solutions containing varying viscosity levels. This experiment showed that the needle gauge affected the pressure required to inject a solution in the SCS. The Hagen-Poiseuille equation was used to calculate the pressure drop in pounds per square inch (PSI) as a function of viscosity for 30 gauge and 29 gauge needles (ISO 9626:2016: regular wall, RW; thin wall, TW; extra thin wall, ETW; and ultra thin wall, UTW, and additional ClearSide (CLSD) needles in design or used in development studies). A conversion factor of PSI=Pa/6894.76 was used to convert to PSI. The total needle length including the mounting length is 14 mm, the injection volume is 0.1 mL, the injection time is modelled at 10 s (Q=0.1 mL/10=0.01 mL/s), and the needle inner diameters considered are: 30 Ga/29 Ga (133 μm ID), 30 Ga TW (165 μm ID), 30 Ga ETW/29 Ga TW (190 μm ID), 30 Ga UTW/29 Ga ETW (240 μm ID), ClearSide (CLSD) brand 30 Ga needle (160 μm ID), CLSD 30 Ga ETW (220 μm ID), CLSD 29 Ga ETW (240 μm ID).

FIGS. 4A-4C show the pressure versus viscosity calculations and Table 3 shows the results tabulated for the preferred (in some cases), target, and limit values. In some cases, the viscosity range can be widened by using larger needles or higher pressure syringes.

Based on the calculations, the preferred (in some cases) formulation viscosity is 34 mPas with a 160 μm ID needle at room temperature while accounting for shear-thinning at injection shear rate. Acceptable viscosity can be up to 103 mPas and 362 mPas for the 160 μm and 220 μm ID needle cases, respectively, for injections that are slower (30 s) or with higher pressure (65 PSI). An acceptable range was calculated to be from 103 mPas, 121 mPas and up to 362 mPas (cP) at room temperature while accounting for shear-thinning at injection shear rate. This is based on a preferred injection pressure target of up to 43 PSI when using CLSD 30 Ga (160 μm ID) needle and 10 s injection. The acceptable range includes injection pressure target using a CLSD 30 Ga ETW (220 μm ID) needle for injection with a pressure target of up to 43 PSI and 10 s injection. Alternatively, an injection pressure target is up to 65 PSI when using CLSD 30 Ga (160 μm ID) needle or a CLSD 30 Ga ETW (220 μm ID) needle, or an injection pressure target is 43 PSI for a 30 s injection. In some cases, the preferred needle is the CLSD 30 Ga (160 μm ID needle) or the CLSD 30 Ga ETW (220 μm ID needle). Other needles are also possible selections, and some may result in even wider viscosity at the injection shear rate values (e.g., up to about 175 mPas for 43 PSI and up to about 250 mPas for 65 PSI over a 10 s injection.

TABLE 3
Viscosity Design Space Based on Injection Pressures

| | Viscosity | Viscosity | Viscosity |
|---|---|---|---|
| | (mPas) | (mPas) | (mPas) |
| | upper range | upper range | upper range |
| | for ≤43 PSI | for 65 PSI | for 100 PSI |
| Needle | (desired) | (target) | (limit) |
| 30 Ga/29 Ga (133 μm | 16 | 24 | 38 |
| ID) | | | |
| 30 Ga TW (165 μm | 38 | 58 | 90 |
| ID) | | | |
| 30Ga ETW/29 Ga | 67 | 102 | 157 |
| TW (190 μm ID) | | | |
| 30 Ga UTW/29 Ga | 171 | 259 | 400 |
| ETW (240 μm ID) | | | |
| CLSD 30 Ga needle | 34 | 51 | 79 |
| (160 μm ID) | | | |
| CLSD 30 Ga ETW | 121 | 183 | 282 |
| (220 μm ID) | | | |
| CLSD 29 Ga ETW | 171 | 259 | 400 |
| (240 μm ID) | | | |

Preferred viscosity values for a 10 s injection and 43 PSI pressure with a 160 μm needle: the preferred design is for a 10 s injection using a needle with an ID of 160 μm. For a 10 s injection using a 160 μm ID needle the preferred viscosity to result in a pressure of 43 PSI at the injection shear rate (calculated to be about 16,000 s⁻¹) is about 34 mPas.

Acceptable viscosity values for a 10 s injection and 43 PSI pressure with a 220 μm needle: for a 10 s injection using a 220 μm ID needle, the acceptable viscosity to result in a pressure of 43 PSI at the injection shear rate (calculated to be about 6,300 s⁻¹) is about 121 mPas.

Acceptable range of viscosity values for a 30 s injection and 43 PSI pressure: a longer injection time of up to 30 s is within an acceptable range. For a 30 s injection using a 160 μm ID needle, the acceptable viscosity to result in a pressure of 43 PSI at the injection shear rate (calculated to be about 5000 s⁻¹) is about 103 mPas. For a 30 s injection using a 220 μm ID needle, the acceptable viscosity to result in a pressure of 43 PSI at the injection shear rate (calculated to be about 2100 s⁻¹) is about 362 mPas.

Acceptable range of viscosity values for a 10 s injection and 65 PSI pressure: a higher pressure of up to 65 PSI is within an acceptable range. For a 10 s injection using a 160 μm ID needle, the acceptable viscosity to result in a pressure of 65 PSI at the injection shear rate (calculated to be about 16,000 s⁻¹) is about 51 mPas. For a 10 s injection using a 220 μm ID needle, the acceptable viscosity to result in a pressure of 65 PSI at the injection shear rate (calculated to be about 6,300 s⁻¹) is about 183 mPas.

Further, the Hagen-Poiseuille equation was used to calculate the pressure drop in pounds per square inch (PSI) as a function of viscosity for 30 gauge and 29 gauge needles. The wider diameter needle (e.g., 29 gauge STW) resulted in lower pressure required for SCS injection. The needle gauge effect on pressure was even more significant as the viscosity of the fluids increased (FIG. 5). For example, a fluid with viscosity of about 30 mPas resulted in a pressure of about 48 PSI when a 30 gauge needle was used, whereas a 48 PSI pressure was observed for a fluid with viscosity of 100 mPas or for a fluid with viscosity of 150 mPas when a gauge STW and a 29 gauge needles were used, respectively (this corresponds to 3.5 times and times the viscosity, respectively) (FIG. 5). Thus, the larger inner diameter needle gauge significantly decreased the resulted pressure.

### 5.4 Example 4: Several Solutions Having Various Excipients were Screened for their Suitability to Increase the Viscosity of the Formulation

For this experiment, different solutions ranging in viscosity were analyzed. The solutions were prepared in a ‘base’ solution also containing 0.2 mg/mL potassium chloride, 0.2 mg/mL potassium phosphate monobasic, 5.84 mg/mL sodium chloride, 1.15 mg/mL sodium phosphate dibasic anyhydrous, 40.0 mg/mL (4% w/v) sucrose, and 0.001% (0.01 mg/mL) poloxamer 188, pH 7.4. The extra viscosity-modifying excipients were dissolved in this solution. Table 4 shows the viscosity measured at low shear using a capillary viscometer, injection pressure into air, and injection pressure into enucleated porcine eyes. During this experiment, the low shear viscosity of PEG3350, Dextran 40k, PEG12000 were considered too low to potentially help increase localization in the suprachoroidal space, so were considered not suitable. In addition, PEG is known to precipitate AAV at higher concentrations so, for example, PEG12000 was not suitable. Polyvinylpyrrolidone appeared to not be sufficiently shear-thinning based on the viscosity and pressure behavior. A search of use of polyvinylpyrrolidone indicated it is primarily used as an excipient for oral delivery and may have safety concerns as a parenteral excipient, so was also eliminated from consideration. Polyvinyl alcohol was very difficult to dissolve and showed signs of foaming and bubbles, so was eliminated based on manufacturing practicality. Variations of polysaccharide excipients were further investigated and analyzed (CMC, Hypromellose, hetastarch) as well as poloxamer 407.

TABLE 4
Measured viscosity and injection pressure into air or enucleated
porcine eyes for screening of excipients to increase viscosity

| | | | | Injection |
|---|---|---|---|---|
| | | | | Pressure |
| | | | Injection | into enu- |
| | Concen- | Viscos- | Pressure | cleated |
| | tration | ity | into air | porcine eye |
| Excipient | (%) | (mPas) | (PSI) | (PSI) |
| water | 0.0 | 1.00116 | 4.7 | 14 |
| Modified DPBS with | 0.0 | 1.33 | 5.0 | NT |
| sucrose buffer control | | | | |
| PEG3350, USP | 10.0 | 3.42 | 12.0 | NT |
| | 6.0 | 2.46 | NT | NT |
| | 3.0 | 1.74 | NT | NT |
| | 1.5 | 1.52 | NT | NT |
| | 0.8 | 1.46 | NT | NT |
| Dextran 40k | 5.0 | 2.59 | 12.3 | NT |
| | 2.5 | 1.91 | 6.7 | NT |
| PEG12000 | 10.0 | 10.94 | 24.0 | NT |
| | 5.0 | 4.70 | 12.7 | NT |
| Carboxymethyl cellulose | 2.0 | 61.77 | 31.0 | NT |
| sodium salt, 10-50 cP | 1.0 | 12.29 | 18.3 | NT |
| (2% H₂O | | | | |
| 25° C.) USP, low | | | | |
| viscosity | | | | |
| Polyvinylpyrrolidone, | 12.0 | 518.03 | 88.0 | NT |
| M.W. ~360,000 K-90 | 6.0 | 99.51 | 54.3 | 38 |
| USP, aka povidone K-91 | 3.0 | 22.36 | 26.7 | NT |
| Polyvinyl alcohol 26-88, | 4.0 | NT | 32.5 | NT |
| EMPROVE ® exp Ph. | | | | |
| Eur., USP, JPE, (4% = | | | | |
| 25 cP) aka Mowiol ® | | | | |
| Hydroxyethyl cellulose, | 4.0 | NT | 48.4 | NT |
| 100 cP NF, aka | 2.0 | NT | 23.7 | NT |
| ‘Hetastarch’ | | | | |
| Carboxymethyl cellulose | 1.0 | NT | 26.9 | 32 |
| sodium salt, 2% = | 0.5 | NT | 18.1 | NT |
| 400-800 cP, USP, | | | | |
| medium viscosity | | | | |
| Hydroxypropyl | 2.0 | NT | 27 | 73 |
| methylcellulose | 1.0 | NT | 24.8 | NT |
| (Hypromellose), 4000 | 0.5 | NT | 15 | 16 |
| mPa · s USP | 0.2 | NT | 11.5 | NT |
| Substitution Type 2910 | 0.1 | NT | 10 | NT |
| poloxamer 407 | 15.0 | NT | 50.2 | 45 |

NT = not tested

### 5.5 Example 5: Several Solutions Having Various Viscosities were Analyzed for Diffusion, Free DNA, and AAV Stability

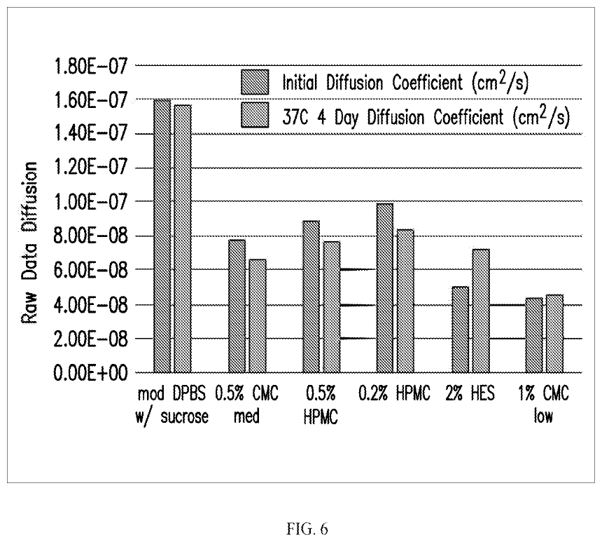

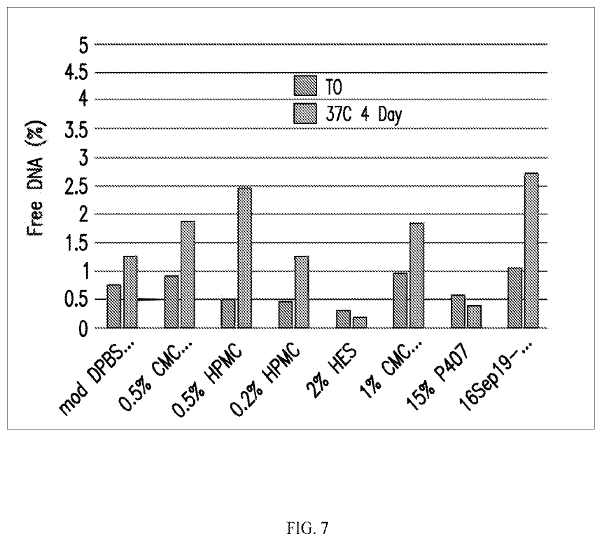

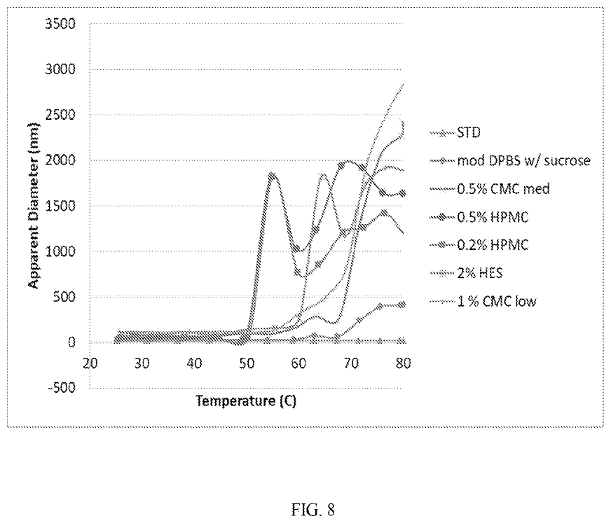

Diffusion data, percent of free DNA, and diameters were analyzed based on data obtained from six solutions having different viscosities. The control solution was DPBS with sucrose. The other five solutions were 0.5% carboxymethyl cellulose (CMC) medium (2%=400-800 cP), 0.5% hydroxypropyl methylcellulose (HPMC), 0.2% HPMC, 2% hydroxyethyl cellulose (HES), and 1% CMC low. In short, an AAV (e.g., a replication deficient adeno-associated viral vector 8 (AAV8) carrying a coding sequence for a soluble anti-VEGF Fab protein) was present in one of the six solutions. FIG. 6 shows the diffusion data obtained for each of the six solutions taken on the initial day (TO) and after four days at 37° C. This experiment showed that the diffusion data was comparable between the initial measurement (TO) and the measurement after four days (FIG. 6). This experiment also confirmed that a diffusion coefficient depends on the viscosity of the solvent. FIG. 7 shows the percentage of free DNA obtained during TO and obtained after four days at 37° C. FIG. 8 shows DLS thermal ramping (DLS-Melt) apparent diameter showing a small peak at about 63° C. followed by a larger melt onset at about 70° C., which is indicative of AAV stability. The HPMC formed gel at 55° C. (FIG. 8). Taken together, the data suggested that CMC and HES solutions were compatible with AAV.

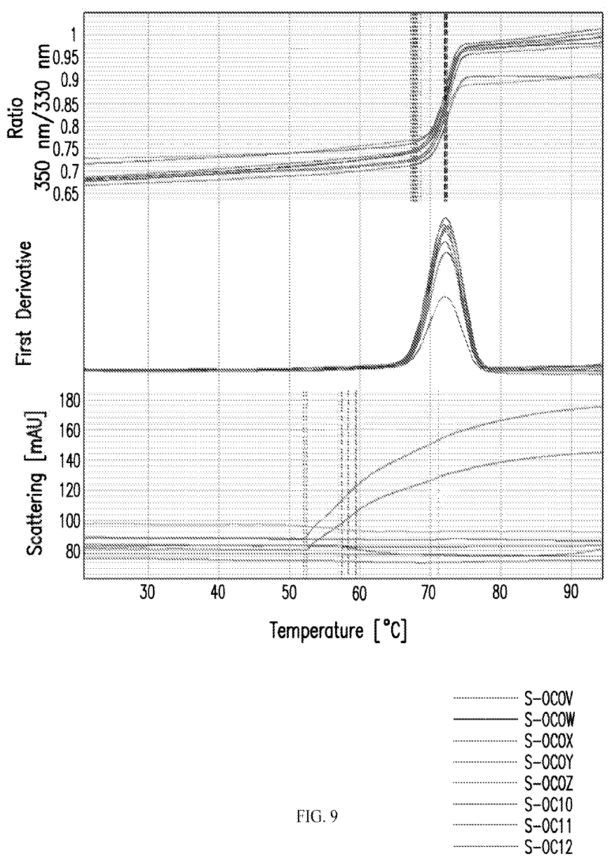

FIG. 9 shows differential scanning fluorimetry thermal ramp data for solutions having different viscosity values. From top to bottom (S-0C0V to S-0C12), the solutions included DPBS with sucrose (control), 0.5% carboxymethyl cellulose medium viscosity grade (CMC) (2%=400-800 cP (or mPas)), 0.5% hydroxypropyl methylcellulose (HPMC), 0.2% HPMC, 2% hydroxyethyl cellulose (HES), and 1% CMC low viscosity grade (2% H₂O 25° C.=10-50 cP), 15% poloxamer 407, and 0.5% carboxymethyl cellulose high viscosity grade (1% H₂O, 25° C.=1500-3000 cP). Top panel: raw melting curve signal. Middle panel: derivative of data to identify the peak. Bottom panel: light scattering data to indicate either aggregation or gel formation. An increase in light scattering due to a hazy gel formation was observed at about 55° C. for the two hypromellose formulations. The melting temperature onset and midpoints shown by vertical lines in the top panel and the peak in the middle panel were similar for all the formulations, demonstrating that the capsids have similar thermal stability in the different formulations. The results are summarized in Table 5.

TABLE 5
Summary of stability of AAV in different viscous formulations as
measured by differential scanning fluorometry melting temperature

Fluorescence Ratio 350 nm/
330 nm Signal Analysis

| | | IP #1 (Melting | |
|---|---|---|---|
| Sample | Sample | tempe- | Tₒₙₛₑₜ |
| description | ID | rature-Tₘ) ° C. | (° C.) |
| control | S-0C0V | 72.40 | 67.84 |
| 0.5% CMCmed | S-0C0W | 72.06 | 67.49 |
| 0.5% HPMC | S-0C0X | 72.34 | 67.89 |
| 0.2% HPMC | S-0C0Y | 72.17 | 67.68 |
| 2% HES | S-0C0Z | 72.05 | 68.58 |
| 1% CMClow | S-0C10 | 72.01 | 67.55 |
| 15% P407 | S-0C11 | 72.30 | 68.17 |
| 0.5% CMChigh | S-0C12 | 71.90 | 67.25 |

Experiments directed to tolerability/safety, stability of AAV, and other delivery factors is performed using various solutions with different viscosities, such as those presented in this experiment. No setbacks arise for the solution containing sucrose and for the majority of the solutions disclosed herein. However, the PEG12000 solution can cause precipitation (causing instability of AAVs), the polyvinylpyrrolidone solution can result in high injection pressure, most likely because it is not shear thinning or has limited shear thinning behavior, and the polyvinyl alcohol solution can be difficult to dissolve.

### 5.6 Example 6: Injection Pressure and Rheology Measurements of Lead Candidate Formulations

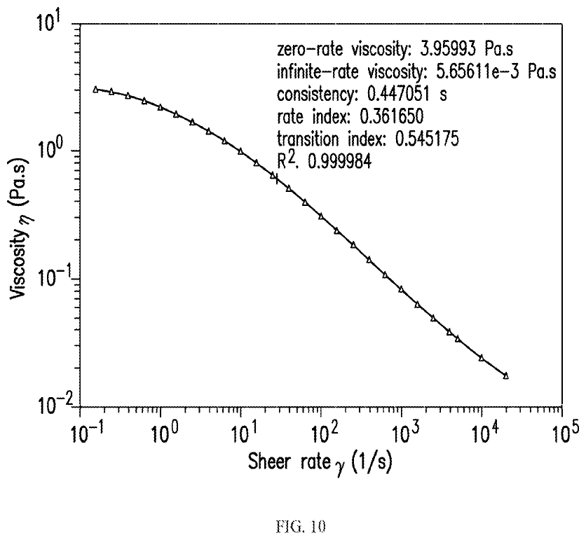

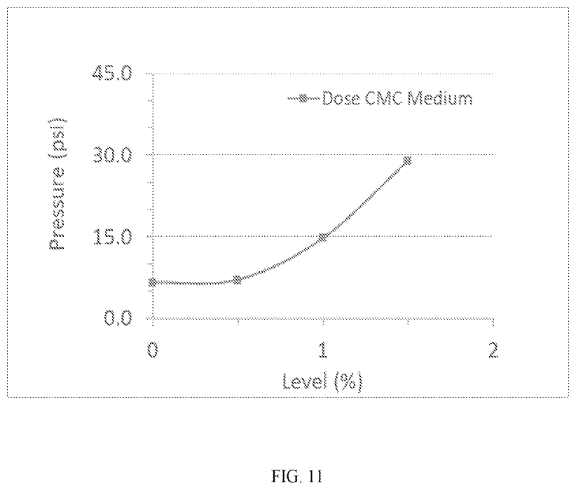

The viscosity versus shear rate for a 1% carboxymethylcellulose high viscosity grade formulation is shown in FIG. 10. The viscosity is greater than 2,000 mPas (2 Pas) at about 1 s⁻¹ shear rate and decreases to a measured value of 34 mPas at 5,000 s⁻¹ and is extrapolated to be as low as about 24 mPas at 10,000 s⁻¹ shear rate. This is a 65-fold decrease in viscosity comparing the viscosity at a shear rate of 1 s⁻¹ to 5000 s⁻¹. Either calculating using the Hagen-Poiseuille equation, or reading the calculated data plot in FIG. 3, this shear-thinning behavior allows a 1% high viscosity grade formulation to be injected with a 30 Ga needle (160 μm ID) with a pressure of between than 40 and 30 PSI if injected over 8 to 10 s. This is well within an acceptable range for an injection pressure while allowing for the formulation to have a very high viscosity once it is injected. For a 30 Ga ETW needle with an ID of 220 μm, the average shear rate for a 10 s injection is about 10,000 s⁻¹, for a 20 s injection is about 5000 As previously described, the preferred (in some cases) maximum viscosity is 34 mPas (using a 160 μm ID needle) or up to 121 mPas (using a 220 μm ID needle) at the injection shear rate.

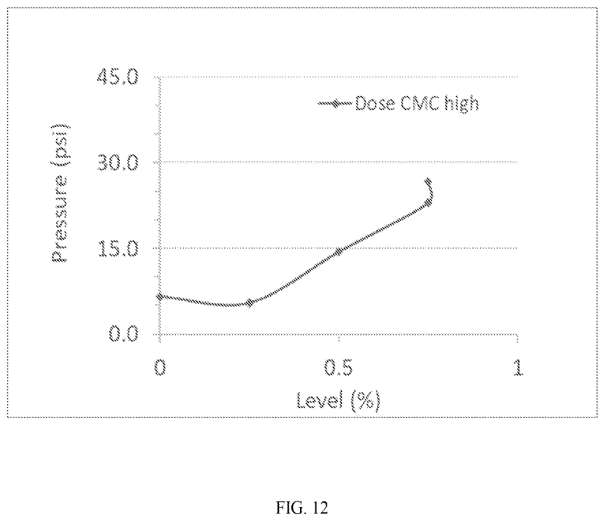

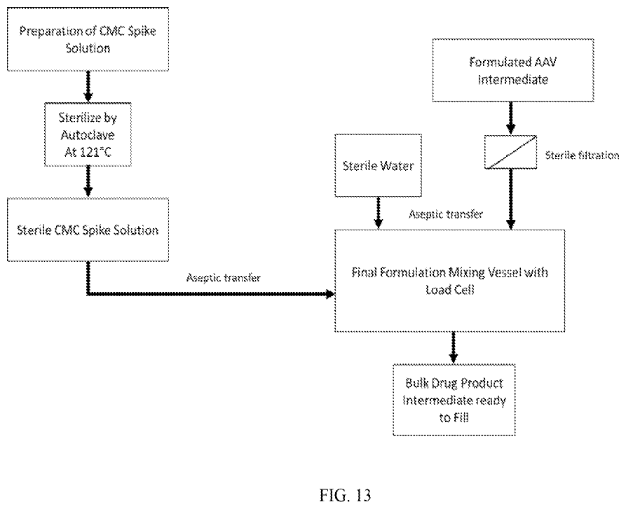

The injection pressure into enucleated porcine eyes for medium viscosity grade carboxymethylecellulose formulations is shown in FIG. 11. FIG. 12 shows the injection pressure into enucleated porcine eyes for high viscosity grade carboxymethylecellulose formulations. The injection pressure did not exceed 43 PSI for either formulation. Based on extrapolation of the data in FIG. 11, using the calculations of pressure drop in FIGS. 4A-4C, and the viscosity data in FIG. 10, a high viscosity grade carboxymethylecellulose formulation, at a level of 1% appears to be suitable for the desired formulation properties. FIG. 13 shows how 1% high viscosity grade carboxymethylecellulose formulation may be manufactured in a sterile manner. For example, 1.11% solution may be prepared, sterilized by autoclave (because filtration of viscous solutions is a challenge), then spiked at a ratio of 9:1 with the formulated AAV intermediate to achieve a final concentration of carboxymethylecellulose of 1%. Water may optionally be added to account for laboratory studies indicating that some slight water loss may occur during the sterilization procedure (about 2-4% loss).

### 5.7 Example 7: Gene Therapy Administered in the Suprachoroidal Space for Subjects with Neovascular Age-Related Macular Degeneration (nAMD)

5.7.1 Brief Summary of Study:

This example relates to a gene therapy treatment for patients with neovascular (wet) age-related macular degeneration (nAMD). In this example, Construct II or a replication deficient adeno-associated viral vector 8 (AAV8) carrying a coding sequence for a soluble anti-VEGF Fab protein, is administered to patients with nAMD using different solutions having different viscosity values (ranging from low viscosity to very high viscosity). The goal of the gene therapy treatment is to slow or arrest the progression of retinal degeneration and to slow or prevent loss of vision with minimal intervention/invasive procedures. Current anti-VEGF therapies have significantly changed the landscape for treatment of wet AMD, becoming the standard of care due to their ability to prevent progression of vision loss in the majority of patients. These therapies, however, require life-long intraocular injections, typically repeated every four to 12 weeks in frequency, to maintain efficacy. Due to the burden of treatment, patients often experience a decline in vision with reduced frequency of treatment over time. Gene therapy administered in the suprachoroidal space is being developed as a potential one-time treatment for wet AMD or for any other ocular disease.

Detailed description of study: this dose-escalation study is designed to evaluate the efficacy, safety, and tolerability of Construct II or AAV8-anti-VEGF-ab gene therapy in subjects with nAMD. Efficacy is the primary focus of the study. Subjects are evaluated for safety and tolerability of Construct II or AAV8-anti-VEGF-ab throughout the study. Approximately 40 subjects who meet the inclusion/exclusion criteria are randomly divided into one of two dose cohorts. Some subjects receive ranibizumab (LUCENTIS®) as a control treatment, some receive Construct II or AAV8-anti-VEGF-ab delivered via one suprachoroidal space (SCS) injection, and some receive Construct II or AAV8-anti-VEGF-ab delivered via two suprachoroidal space (SCS) injections. This example can also be used to deliver gene therapy present in different solutions having varying viscosity levels. For example, some solutions can have high viscosity while others can have low viscosity. The efficacy, safety and tolerability of Construct II or AAV8-anti-VEGF-ab (or any other gene therapy) can also be analyzed in relation to the viscosity of the delivery solutions.

The primary outcome measure of this study is to evaluate the mean change in best corrected visual acuity (BCVA) for Construct II or AAV8-anti-VEGF-ab compared with ranibizumab monthly—over a time frame of 40 weeks. The scale used is the early treatment diabetic retinopathy study (ETDRS) letter score from 0-100 (higher score being better vision).

The Secondary outcome measures of this study includes: 1) evaluating the safety and tolerability of Construct II or AAV8-anti-VEGF-ab by detecting the incidences of ocular and non-ocular adverse events (AEs) and of serious adverse events (SAEs) over a time frame of 52 weeks; 2) evaluating the effect of Construct II or AAV8-anti-VEGF-ab on choroidal neovascularization (CNV) lesion growth and leakage over a time frame of 52 weeks by analyzing the mean change from baseline in CNV lesion size and leakage area based on fluorescein angiography (FA) at week 40 and week 52; 3) evaluating the effect of Construct II or AAV8-anti-VEGF-ab on BCVA over a time frame of 52 weeks by analyzing the mean change from baseline in BCVA to week 52; 4) evaluating the effect of Construct II or AAV8-anti-VEGF-ab on central retinal thickness (CRT) over a time frame of 52 weeks by analyzing the mean change from baseline in CRT as measured by spectral domain optical coherence tomography (SD-OCT) to week 40 and week 52; and 5) assessing the need for supplemental anti-vascular endothelial growth factor (VEGF) therapy in participants who receive Construct II or AAV8-anti-VEGF-ab treatment over a time frame of 52 weeks (e.g., by checking the annualized supplemental anti-VEGF injection rate through week 40 and week 52).

5.7.2 Eligibility Criteria:

The following eligibility criteria apply to this study:
Minimum Age: 50 years
Maximum Age: 89 years
Sex: All
Gender Based: No
Accepts Healthy Volunteers: No

5.7.3 Inclusion Criteria:

Patients ≥50 years and ≤89 years with a diagnosis of subfoveal CNV secondary to age-related macular degeneration (AMD) in the study eye.

Participants must have demonstrated a meaningful response to anti-VEGF therapy.

5.7.4 Exclusion Criteria:

CNV or macular edema in the study eye secondary to any causes other than AMD.

Subfoveal fibrosis or atrophy in study eye.

Subjects who have had a prior vitrectomy.

Any condition in the investigator's opinion that could limit visual acuity (VA) improvement in the study eye.

Active or history of retinal detachment in the study eye.

Uncontrolled glaucoma in the study eye.

Received any gene therapy.

Any condition preventing visualization of the fundus or VA improvement in the study eye, e.g., cataract, vitreous opacity, fibrosis, atrophy, or retinal epithelial tear in the center of the fovea.

History of intraocular surgery in the study eye.

Receipt of any investigational product within 30 days of Visit 2.

Myocardial infarction, cerebrovascular accident, or transient ischemic attacks within 6 months of study entry.

### 5.8 Example 8: Comparison of Suprachoroidal and Subretinal Injection of AAV (e.g., AAV8-antiVEGFfab) in Animal Models

5.8.1 Brief Summary of the Study

The following studies are conducted to compare the expression achieved by suprachoroidal versus subretinal injections of various pharmaceutical compositions (e.g., liquid formulation) containing an AAV (e.g., a replication deficient adeno-associated viral vector 8 (AAV8) carrying a coding sequence for a soluble anti-VEGF Fab protein). This experiment is also used to determine whether suprachoroidal injection of an AAV (e.g., AAV8-antiVEGFfab) in a medium to high viscous solution can reduce VEGF-induced leakage and neovascularization in the eye and produce increased anti-VEGF as compared to the delivery of an AAV in a low viscous solution by a subretinal injection or by a suprachoroidal injection. Several pharmaceutical compositions (e.g., liquid formulation) containing different viscosity levels are tested.

Results show that the viscosity of the solutions affects the antiVEGFfab detected in the eyes injected with suprachoroidal or subretinal AAV-antiVEGFfab, affects the anti-VEGF protein distribution in the retina vs. choroid, and affects the neutralizing VEGF-induced leakage and neovascularization. The concentration of antiVEGFfab protein is measured by ELISA to demonstrate that an AAV-antiVEGFfab delivered in the presence of a more viscous solution (compared to PBS or compared to a solution commonly used for AAV subretinal injections) results in a higher level of antiVEGFfab detected in the eyes as compared to an AAV-antiVEGFfab delivered in the presence of a control solution (e.g., PBS, a solution commonly used for AAV subretinal injections, or a less viscous solution), when the solutions are injected in the SCS (suprachoroidal injection). This experiment also shows that a higher level of antiVEGFfab is detected in the eyes when a more viscous solution containing an AAV-antiVEGFfab is injected via a suprachoroidal injection as compared to a subretinal injection of an AAV-antiVEGFfab in a control solution (e.g., a solution commonly used for AAV subretinal injection, or a less viscous solution). This experiment also shows that a higher level of antiVEGFfab is detected in the eyes when a viscous pharmaceutical composition containing an AAV-antiVEGFfab is injected via a suprachoroidal injection as compared to when the same viscous pharmaceutical composition is administered by subretinal injection. The same concentration of viral genome is used for the SCS and the subretinal administrations.

Vascular leakage is assessed by measurement of albumin in vitreous samples by ELISA to demonstrate that a suprachoroidal injection of a viscous solution containing an AAV-antiVEGFfab is more effective at neutralizing VEGF-induced leakage and neovascularization as compared to a subretinal injection of the same viscous solution containing the AAV-antiVEGFfab.

5.8.2 Methods

Animals (e.g., Norway Brown rats) receive suprachoroidal or subretinal injection of, for example, 3 μl containing 2.85×10¹⁰ genome copies (GC) per eye (concentration of 4×10¹⁰ GC/ml) of AAV8-CB7-antiVEGFfab in one eye, and a suprachoroidal or subretinal injection of 3 μl containing 7.2×10⁸ GC of AAV8-CB7-GFP in the other eye. After a few weeks (e.g., 2 weeks), VEGF (e.g., 200 ng) is injected into the eyes. In a subset of animals, different amounts of VEGF (e.g., 100 ng) is injected.

5.8.3 Results

Fundus photographs (e.g., at 2 weeks) taken 24 hours after the VEGF injection show normal retinas and retinal vessel caliber in the AAV8-antiVEGFfab-injected eyes, whereas the AAV8-GFP-injected eyes show dilated vessels, evidence of edema, blurred optic disc margins and opalescent retina.

Vascular leakage is assessed by measuring albumin in vitreous samples by ELISA. Higher levels of antiVEGFfab is detected in eyes injected with a viscous solution containing AAV8-antiVEGFfab in the SCS as compared when the same pharmaceutical composition is injected via subretinal administration using the same concentration of viral genome. Also, higher levels of antiVEGFfab is detected in eyes injected with a more viscous solution containing AAV8-antiVEGFfab in the SCS as compared to a control solution (e.g., a solution normally used for AAV subretinal injections, or a less viscous solution) containing AAV8-antiVEGFfab injected in the SCS or injected via subretinal delivery. The AAV antiVEGFfab remains in the site of injection (spread less) and is more localized when a more viscous solution is used to inject the AAV in the SCS as compared to when a control solution (e.g., a solution normally used for AAV subretinal injections, or a less viscous solution) is used to inject the AAV in the SCS or via subretinal delivery.

### 5.9 Example 9: Effect of Liquid Formulation on Suprachoroidal Space (SCS) Thickness

The effect of liquid formulation on SCS thickness and the SCS collapse rate over time is measured in living animals (e.g., rabbit, mouse, or monkey). Different solutions having different viscosities are used. Examples of solutions that can be used in this experiment are disclosed in the present disclosure, such as in Example 1. Solutions containing, for example, different percentages of CMC or HES can be used in this experiment. The initial SCS thickness at the injection site is calculated for the various pharmaceutical compositions (e.g., liquid formulation), by for example, using an ultrasound imaging (see Section 4.6). The SCS thickness (e.g., SCS thickness measured before injection and after injection) depends on the viscosity of the solutions. The SCS thickness can be measured at different time points, such as, at different time points before injection and after injection. For example, a 5% CMC solution shows a higher SCS thickness as compared to a 1% CMC solution or PBS. The SCS thickness is also measured over time at different positions in the eye. The viscosity of the solutions impact the thickness of the SCS over time. For example, a 1% CMC solution increases the SCS thickness near the site of injection even when measured over time, while the SCS thickness at the injection site decreases over time when PBS solution is used. The decrease in SCS thickness at the injection site over time when using PBS, is accompanied by a concomitant increase in SCS thickness at adjacent sites in the SCS. The viscosity of the liquid impacts the duration of the SCS thickness and the localization of the SCS thickness. The viscosity of the solution also impacts the amount of time it takes for the solution to be cleared from the SCS. For example, solutions having 1% CMC remain in the SCS (or in the eye) for a longer period of time as compared to a low viscosity solution, such as PBS.

### 5.10 Example 10: Ultrasound Imaging to Determine Suprachoroidal Space (SCS) Thickness

A high-frequency ultrasound (U/S) probe (e.g., UBM Plus, Accutome, Malvern, PA) is used to generate 2D cross-sectional images of the SCS in eyes (e.g., animal eyes) ex vivo (see Section 4.6). The cross-sectional images are generated after the eyes are injected with a solution. The solution can range in viscosity and volume. For example the volume can range from 1 μL to 500 μL. In some cases, the volume can be less than 1 μL or more than 500 μL. The solution can be an aqueous solution (e.g., water), PBS, Hank's Balanced Salt Solution (HB SS), 1%-5% CMC, or any other solution of the present disclosure. The solution can further include a dye (e.g., a fluorescent dye, red-fluorescent, blue-fluorescent, blue dye, or any other dye). The solution can also include any composition, drug, agent, or virus (e.g., AAV), that can be used with the present disclosure. An U/S probe cover (e.g., Clearscan, Eye-Surgical-Instruments, Plymouth, MN) is attached to the UBM Plus to facilitate U/S image acquisition. A few minutes after injection, the U/S probe is used to acquire sagittal views around the eye (e.g., at positions 12, 1.5, 3, 4.5, 6, 7.5, 9, and 10.5 o'clock). Post-processing of the U/S B scans is performed to find the thickness from the outer sclera to the inner retina (e.g., at 1, 5, and 9 mm) posterior to the scleral spur. The mean, median, and standard deviation for each eye is calculated. Calculation of SCS thickness in ultrasound B scans can be performed by, for example, finding a line segment perpendicular to the sclera and choroid, from the outer sclera to the inner retina. The conjunctiva is excluded from the measurement. The tissue thickness is found and subtracted out, resulting in the SCS thickness.

### 5.11 Example 11: Treatment of Batten-CLN1 or CLN2-Associated Vision Loss by Suprachoroidal Injection

A subject presenting with Batten-CLN1-associated vision loss is administered AAV8 or AAV9 that encodes Palmitoyl-Protein Thioesterase 1 at a dose sufficient to produce a therapeutically effective concentration of the transgene product in the eye (e.g., vitreous humor) for three months. A subject presenting with Batten-CLN2-associated vision loss is administered AAV8 or AAV9 that encodes Tripeptidyl-Peptidase 1 at a dose sufficient to produce a therapeutically effective concentration of the transgene product in the eye (e.g., vitreous humor) for three months. The administration is done by administration to the suprachoroidal space. Several pharmaceutical compositions (e.g., liquid formulation) having different viscosity are used. The viscosity of the pharmaceutical compositions (e.g., liquid formulation) impact Batten-CLN2 or CLN1-associated vision loss and efficacy of treatment. Following treatment, the subject is evaluated for improvement in Batten-CLN2-associated vision loss. Following treatment, the subject is evaluated for improvement in Batten-CLN1-associated vision loss. Subjects that have the AAV administered in the SCS when a viscous pharmaceutical composition is used show better improvement in Batten-CLN1 or CLN2-associated vision loss as compared to subjects that have the same pharmaceutical composition administered by subretinal injection. Subjects that have the AAV administered in the SCS when a relatively viscous pharmaceutical composition is used show better improvement in Batten-CLN1 or CLN2-associated vision loss as compared to subjects that have a reference pharmaceutical composition administered by subretinal injection, by intravitreous administration, or to the SCS.

Effects of the methods provided herein on visual deficits are measured by one or more visual acuity screenings, including OptoKinetic Nystagmus (OKN). OKN visual acuity screening uses the principles of the OKN involuntary reflex to objectively assess whether a patient's eyes can follow a moving target. The percentage change in OKN screening results before and after the said treatment is calculated.

### 5.12 Example 12: Use of an Infrared Thermal Camera to Monitor Injection in Human Patients

A subject presenting with wet AMD is administered AAV8 that encodes ranibizumab Fab (e.g., by subretinal administration, suprachoroidal administration, or intravitreal administration) at a dose sufficient to produce a concentration of the transgene product at a Cmin of at least 0.330 μg/mL in the eye (e.g., vitreous humor) for three months. The AAV8 encoding ranibizumab Fab can be administered using several pharmaceutical compositions (e.g., liquid formulation) that have different viscosity, by suprachoroidal administration. Subjects that have the AAV8 encoding ranibizumab Fab administered in a medium to high viscosity solution (compared to PBS or compared to a solution commonly used for AAV subretinal injections) show a higher concentration of the transgene (e.g., as measured at 1 week, 2 weeks, 3 weeks, 4 weeks, 8 weeks, or 12 weeks after administration) as compared to the concentration of the transgene in subjects that have the AAV8 encoding ranibizumab Fab administered in a low viscosity solution (e.g., PBS, or a solution commonly used for AAV subretinal administration, or a less viscous solution) by suprachoroidal administration. The concentration of the transgene can be measured at any time after administration of AAV8 encoding ranibizumab Fab. For example, subjects that have the AAV8 administered in the SCS using a more viscous solution show a higher concentration of the transgene in the eye as compared to subjects that have the AAV8 administered in the SCS, or via subretinal administration, or via an intravitreous administration using a less viscous solution as measured at 1 week, 4 weeks, 2 months, or 3 months after administration of the AAV. Similarly, subjects that have the AAV8 administered in the SCS using a viscous solution show a higher concentration of the transgene as compared to subjects that have the same pharmaceutical composition administered via subretinal administration or via intravitreous administration. All solutions that are used in this experiment have the same amount of genome copies.

An FLIR T530 infrared thermal camera is used to evaluate the injection during the procedure and is available to evaluate after the injection to confirm either that the administration is successfully completed or misdose of the administration. Alternatively, an FLIR T420, FLIR T440, Fluke Ti400, or FLIRE60 infrared thermal camera is used. Following treatment, the subject is evaluated clinically for signs of clinical effect and improvement in signs and symptoms of wet AMD.

### 5.13 Example 13: Components in Formulation A and Formulation B

This example shows the components in Formulation A (Dulbecco's phosphate buffered saline with 0.001% poloxamer 188, pH 7.4), stored at ≤−60° C., and Formulation B (‘modified Dulbecco's phosphate buffered saline with 4% sucrose and 0.001% poloxamer 188, pH 7.4’), stored at −20° C. The comparison and impact analysis for the two Formulations is provided in Table 6. Formulation B has improved storage feasibility, without impact on the AAV product observed to date after 2 years of storage. Other pharmaceutical compositions (e.g., liquid formulation) having different viscosity values are tested. Pharmaceutical compositions of the present disclosure (e.g., with medium or high viscosity) can include, for example, one or more components from Formulation B. Pharmaceutical compositions of the present disclosure (e.g., with medium or high viscosity) have improved storage feasibility, without impact on the AAV product (e.g., after 2 years of storage).

TABLE 6
Formulations A and B

| Process Site/Stage | Formulation A | Formulation B |
|---|---|---|
| Formulation | DPBS with 0.001% Poloxamer | ‘modified DPBS with 4% Sucrose and 0.001% |
| Buffer | 188, pH 7.4. | Poloxamer 188, pH 7.4.’ |
| | Composition: | The ‘modified DPBS with 4% Sucrose and |
| | 0.2 mg/mL potassium chloride, | 0.001% Poloxamer 188, pH 7.4.’ formulation |
| | 0.2 mg/mL potassium phosphate | has 4% w/v of sucrose and a lower sodium |
| | monobasic, 8.1 mg/mL sodium | chloride level (reduced from 137 mM to |
| | chloride, 1.15 mg/mL sodium | 100 mM) to compensate tonicity. The other |
| | phosphate dibasic anyhydrous, | formulation excipients and levels are |
| | 0.001% (0.01 mg/mL) poloxamer | identical. |
| | 188, pH 7.4 | Composition: 0.2 mg/mL potassium chloride, |
| | | 0.2 mg/mL potassium phosphate monobasic, |
| | | 5.84 mg/mL sodium chloride, 1.15 mg/mL |
| | | sodium phosphate dibasic anyhydrous, 40.0 |
| | | mg/mL (4% w/v) sucrose, 0.001% (0.01 mg/mL) |
| | | poloxamer 188, pH 7.4 |
| FDP Long-term | ≤−60° C. | ≤−20° C. |
| Frozen Storage | | |
| Temperature | | |

Formulation B (Modified DPBS with Sucrose) includes 0.2 mg/mL potassium chloride, 0.2 mg/mL potassium phosphate monobasic, 5.84 mg/mL sodium chloride, 1.15 mg/mL sodium phosphate dibasic anyhydrous, 40.0 mg/mL (4% w/v) sucrose, 0.001% (0.01 mg/mL) poloxamer 188, pH 7.4. In molar units, Formulation B includes 2.70 mM potassium chloride, 1.47 mM potassium phosphate monobasic, 100 mM sodium chloride, 8.1 mM sodium phosphate dibasic anyhydrous, 117 mM sucrose, 0.001% (0.01 mg/mL) poloxamer 188, pH 7.4. The density of Formulation B may be 1.0188 g/mL; The osmolality of Formulation B may be approximately 345 (331-354).

TABLE 7
Formulation B with Construct II as Active Pharmaceutical Ingredient (API)

| | | | Concen- | Concen- | | Mass | Vendor | | Molecular |
|---|---|---|---|---|---|---|---|---|---|
| | | Quality | tration | tration | | Fraction | and Part | Chemical | Weight |
| Ingredient | Function | Standard | (mg/mL) | (mM or %) | | (g/kg)^{b} | Number | Formula | (g/mol) |
| Construct II | API | Internal | Varies based | — | | — | — | — | — |
| | | | on dose level | | | | | | |
| Sodium | Buffering | USP, | 5.84 | 100 | mM | 5.736 | Avantor, | NaCl | 58.440 |
| Chloride | Agent | Ph. Eur, | | | | | 3627 | | |
| | | BP, JPE | | | | | | | |
| Potassium | | USP, BP, | 0.201 | 2.70 | mM | 0.198 | Avantor, | KCl | 74.5513 |
| Chloride | | Ph. Eur, | | | | | 3045 | | |
| | | JPE | | | | | | | |
| Sodium | | USP, Ph. | 1.15 | 8.10 | mM | 1.129 | Avantor, | Na₂HPO₄ | 141.960 |
| Phosphate | | Eur, JPE | | | | | 3804 | | |
| Dibasic | | | | | | | | | |
| Anhydrous | | | | | | | | | |
| Potassium | | NF, BP, | 0.200 | 1.47 | mM | 0.196 | Avantor, | KH₂PO₄ | 136.086 |
| Phosphate | | Ph. Eur | | | | | 3248 | | |
| Monobasic | | | | | | | | | |
| Sucrose | Cryoprotectant | USP, NF, | 40.0 | 117 | mM | 39.26 | Pfanstiehl, | C12H22O11 | 342.3 |

| | | | | | | | | |
|---|---|---|---|---|---|---|---|---|
| | | Ph. Eur, | | | | S-124-2-MC | | |
| | | BP, JPE | | | | | | |
| Poloxamer | Surfactant^{a} | NF, Ph. | 0.010 | 0.001% | 0.1 | BASF, | HO(C₃H₆O)ₐ(C₂H₄O)_{b}(C₃H₆O)ₐH | 7680 to |
| 188 | | Eur, JPE | | | mL/kg of | 50424596 | | 9510 |
| | | | | | 10% | | | |
| | | | | | stock | | | |
| Water | Aqueous | WFI | Approximately | Approximately | QS to 1 | Varies | H₂O | 18.0153 |
| | Vehicle | | 971 mg/mL | 54M | kg | | | |
| | | | | | (need | | | |
| | | | | | approx. | | | |
| | | | | | 953 g/kg) | | | |

^{a}Spike 0.1 mL/L = 0.1 mL/kg of 10% stock P188. NF grade Pluronic ® F-68 (poloxamer 188) from Spectrum and Kolliphor ® P188 BIO from BASF may be used.
^{b}Volume of 1 kg of solution is approximately 982 mL (1 kg/1.0188 kg/L = 982 mL)

### 5.14 Example 14: Comparison of Formulation A and Formulation B in Long Term Stability

This example shows the comparison of Formulation A and Formulation B in long term stability. Formulation A and B had similar long-term frozen stability at −80° C., and Formulation B was also stable at −20° C. The ‘modified dPBS with 4% sucrose’ formulation B maintained potency for 12 months at −20° C. and −80° C. Other pharmaceutical compositions (e.g., liquid formulation) having different viscosity values are tested. Pharmaceutical compositions of the present disclosure (e.g., with medium or high viscosity) are stable at −20° C. and at −80° C. Pharmaceutical compositions with medium and high viscosity maintains potency for 12 months at −20° C. and −80° C. Pharmaceutical compositions of the present disclosure (e.g., with medium or high viscosity) can include, for example, one or more components from Formulation B.

### 5.15 Example 15: Comparison of Formulation a and Formulation C In-Vitro Potency

This example shows the comparison of Formulation A and Formulation C in long term stability. Formulation C is a variant of the ‘modified dPBS with sucrose’ with 60 mM NaCl and 6% sucrose. Formulation C includes 0.2 mg/mL potassium chloride, 0.2 mg/mL potassium phosphate monobasic, 3.50 mg/mL sodium chloride, 1.15 mg/mL sodium phosphate dibasic anyhydrous, 60.0 mg/mL (6% w/v) sucrose, 0.001% (0.01 mg/mL) poloxamer 188, pH 7.4.

Formulation C was stable for 2 years at −20° C. The reference formulation A (dPBS) was not stable at −20° C. Formulations B and C may have comparable and superior long-term stability at −20° C. Other pharmaceutical compositions (e.g., liquid formulation) having different viscosity values are tested. Pharmaceutical compositions of the present disclosure (e.g., with medium or high viscosity) can include, for example, one or more components from Formulation B or Formulation C. Pharmaceutical compositions of the present disclosure (e.g., with medium or high viscosity) are stable for 2 years at −20° C.

### 5.16 Example 16: Pharmacodynamic, Biodistribution, and Tolerability Study in Cynomolgus Monkeys Using Different Suprachoroidal Formulations

The objective of this study is to evaluate the biodistribution, pharmacodynamics (transgene concentration), and tolerability of different formulations comprising AAV8-anti-VEGF-ab when administered as a single dose via suprachoroidal injection to Cynomolgus monkeys. After dosing, animals are observed postdose for at least 4 weeks. One group is also administered a high volume of the formulations. Some of the formulations include varying viscosity levels, ranging from low viscosity to high viscosity. For example, Formulation 1 has low viscosity, Formulation 2 has intermediate viscosity, and Formulation 3 has high viscosity. The group assignment and dose levels are shown in Table 8. The test article is AAV8-anti-VEGF-ab. The control article is a placebo. The formulations and the controls can be stored in a freezer between −60° C. and −80° C. and thawed at room temperature on the day of use, or stored at room temperature if used on the day of formulation, or stored in a refrigerator between 2° C. and 8° C. The indication is chronic retinal conditions including wet AMD and diabetic retinopathy.

TABLE 8
Group Assignment and Dose Levels

| | | | Dose | Dose | Number |
|---|---|---|---|---|---|
| | Dose | Dose | level^{b} | Concen- | of |
| | Regimen | Regimen | (GC/ | tration | Animals |
| Group | Left Eye | Right Eye | eye) | (GC/mL) | (Females) |
| Control 1^{a} | Control | Control | 0 | 0 | 41 |
| | Article 1 | Article 1 | | | |
| Control 2 | Control | Control | 0 | 0 | 1 |
| | Article 2 | Article 2 | | | |
| Control 3 | Control | Control | 0 | 0 | 1 |
| | Article 3 | Article 3 | | | |
| Formu- | Test | Test | 3 × 10¹¹ | 3 × 10¹² | 4 |
| lation 1 | Article 1 | Article 1 | | | |
| Formu- | Test | Test | 3 × 10¹¹ | 3 × 10¹² | 4 |
| lation 2 | Article 2 | Article 2 | | | |
| Formu- | Test | Test | 3 × 10¹¹ | 3 × 10¹² | 4 |
| lation 3 | Article 3 | Article 3 | | | |
| High | Test | Test | 3 × 10¹¹ | 1.5 × 10¹² | 4 |
| volume | Articles 1, | Article 1, | | | |
| formu- | 2, or 3 | 2, or 3 | | | |
| lation | | | | | |

GC = Genome copies
^{a}Group 1 will be administered control article only.
^{b}Dose levels are based on a dose volume of 100 μL/eye for Formulations 1-3, and volume of 200 μL/eye for the high volume formulation group. Each eye is administered two injections.
c all animals are sacrificed on day 29 of the dosing phase.

Antibody Prescreening at Animal Supplier: blood (at least 1 mL) from about 90 female monkeys is collected from each animal via a femoral vein and placed into tubes containing no anticoagulant. Another vein may be used for collection, as needed. Animals are selected as study candidates based on the pre-screening results. Blood is allowed to clot at room temperature and centrifuged within 1 hour to obtain serum. Serum is divided into 2 aliquots and placed into cryovials and maintained on dry ice prior to storage at approximately −70° C. Samples are shipped overnight on dry ice for analysis. Samples are then analyzed for anti-AAV8 neutralizing antibodies (NAbs) by any acceptable method. Animals are selected for shipment based on anti-AAV8 Nab results.

Dose Administration: animals are fasted overnight and anesthetized with ketamine and dexmedetomidine prior to suprachoroidal injection. In brief, a single suprachoroidal injection of 100 μL (or 2 injections of 50 μL each) is administered to each eye (between 3 and 4 mm from the limbus) over 5 to 10 seconds. For the high volume formulation, 200 μL per eye is administered. The formulations are administered with Clearside SCS Microinjectors. The microneedle size varies depending on the viscosity of the formulation. In some cases a 30-gauge microneedle is used. Injections in the right eye is administered in the superior temporal quadrant (i.e., between the 10 o'clock and 11 o'clock positions. Injections in the left eye is administered in the superior temporal quadrant (i.e., between the 1 o'clock and 2 o'clock positions). Following the injection, the needle is kept in the eye for approximately 5 seconds before being withdrawn. Upon withdrawal of the micro needle, a cotton-tipped applicator (dose wipe) is placed over the injection site for approximately 10 seconds. A topical antibiotic (e.g. Tobrex® or appropriate substitute) is instilled in each eye following dosing. Each dosing time is recorded as the time at the completion of each injection. The right eye is dosed first, followed by the left eye.

Ophthalmic Procedures: ophthalmic examinations (e.g., on days 4, 8, 15, and 29 post administration) are conducted. Animals are examined with a slit lamp biomicroscope and indirect ophthalmoscope. The adnexa and anterior portion of both eyes are examined using a slit lamp biomicroscope. The ocular fundus of both eyes are examined (where visible) using an indirect ophthalmoscope. Prior to examination with the indirect ophthalmoscope, pupils are dilated with a mydriatic agent (e.g., 1% tropicamide). Intraocular pressure is measured on the day of administration (within 10 minutes prior to dosing) and, for example, on days 4, 8, 15, and 29. Rebound tonometry (TonoVet) can be used to evaluate ocular pressure. Ocular photography is performed around week 4. Photographs are taken with a digital fundus camera. Color photographs are taken of each eye to include stereoscopic photographs of the posterior pole and nonstereoscopic photographs of two midperipheral fields (temporal and nasal). Photographs of the periphery is also performed. Further, autofluoresence imaging with indocyanine green is conducted to document spread of dose (e.g., on days one and two).

Anti-AAV8 Neutralizing Antibody Analysis: blood samples from each animal taken from a femoral vein at different time points (e.g., prior to administration, on day of administration, and on days after administration) are held at room temperature and allowed to clot for at least 30 minutes prior to centrifugation. Samples are centrifuged within 1 hour of collection, and serum is harvested. Following harvesting, samples are placed on dry ice until stored between −60° C. and −80° C. Serum analysis for AAV8 antibodies is then performed using a qualified neutralizing antibody assay.

Anti-AAV8-anti-VEGF-ab Transgene Product Antibody Analysis: blood samples are taken as discussed above and serum samples are analyzed for antibodies to the AAV8-anti-VEGF-ab using any assay of the present disclosure or any acceptable assay. For AAV8-anti-VEGF-ab transgene analysis, blood samples are taken as described above at least two weeks prior to administration, on day 15, and on the day of animal sacrifice (Day 29). 50 μL from the anterior chamber is collected before dose administration. Samples from the aqueous humor and the vitreous humor can be collected at the terminal necropsy. Serum samples can be collected pre-dose, on Day 15, and prior to necropsy. Samples are then analyzed by any assay of the present disclosure or any applicable assay or method (e.g., for transgene concentration).

Aqueous Humor Collection: approximately 50 μL is removed from each eye at least 2 weeks prior to administration, on day 15, and on the day the animals are sacrificed. Aqueous humor samples from each eye is placed into separate tubes with Watson barcoded labels, snap frozen in liquid nitrogen, and placed on dry ice until stored between −60° C. and −80° C.

Post-Aqueous Tap Medication Regimen: the objective of this treatment regimen is to provide palliative treatment related to aqueous humor collection procedures. The treatment objective following collection days is to provide appropriate palliation of adverse events (e.g., discomfort). Animals are tested for ocular pain and side effects.

TABLE 9
Medication Regimen

| | | Dose | |
|---|---|---|---|
| Days | Drug (Dose Level) | Route | Interval |
| Day of sampling | Flunixin meglumine | IM | Prior to sedation for |
| | (2 mg/kg) | | collection |
| Day of sampling | Buprenorphine (0.05 | IM | Upon recovery from |
| | mg/kg) | | anesthesia; 5 to 7 |
| | | | hours later, and at |
| | | | least 16 hours later |
| | | | (from the first |
| | | | injection) |
| Day of sampling | 1% Atropine sulfate | Topical | After collection |
| | solution^{a} | | procedures |
| Day of sampling | Neo-Poly-Dex | Topical | After collection |
| | ointment^{b} | | procedures |
| 1 day after | 1% Atropine sulfate | Topical | Once |
| sampling | solution^{a} | | |
| 1 day after | Neo-Poly-Dex | Topical | BID |
| sampling | ointment^{b} | | |
| 2 days after | 1% Atropine sulfate | Topical | Once |
| sampling | solution^{a} | | |
| 2 days after | Neo-Poly-Dex | Topical | BID |
| sampling | ointment^{b} | | |

BID = Twice daily (at least 6 hours apart);
IM = Intramuscular injection
^{a}Applied as 1 to 2 drops of solution to each eye from which samples were collected.
^{b}Applied as an approximate 0.25 inch strip to each eye from which samples were collected.

Termination of Study: animals are anesthetized with sodium pentobarbital and exsanguinated on Day 29.

Necropsy Collections of Aqueous Humor and Vitreous Humor: up to 50 μL per eye and up to 100 μL per eye is removed from the aqueous humor and the vitreous humor, respectively. Following exsanguination, eyes are enucleated and aqueous humor and vitreous humor samples are collected from each eye. Vitreous humor samples are divided into 2 approximately equal aliquots and aqueous humor samples are stored as one aliquot. After each collection, the right eyes of animals are injected with modified Davidson's fixative until turgid. Eyes are stored in modified Davidson's fixative for 48 to 96 hours, and then transferred to 10% neutral-buffered formalin. Samples are flash frozen and stored between −60° C. and −80° C. Aqueous and vitreous samples are analyzed for transgene concentration.

Ocular Tissue Collection for Biodistribution: following exsanguination, the left eye from all animals and right eye from two animals (depending on survival) from the various formulation groups are enucleated and tissues are collected. Tissues are collected into separate tubes with Watson barcoded labels. Collected tissue includes choroid with retinal pigmented epithelium, cornea, iris-ciliay body, optic chiasm, optic nerve, retina, sclera, and posterior eye cup. Eyes are divided into four approximately equal quadrants (superior-temporal to include the area of the dose site, superior-nasal, inferior-temporal, and inferior nasal to include the area of the dose site). From each quadrant, one sample is taken using an 8 mm biopsy punch. Samples are stored between −60° C. and −80° C. Samples are analyzed for vector DNA or RNA using a qPCR or qRT-PCR method.

Non-Ocular Tissue Collection for Biodistribution: two samples of approximately 5 mm×5 mm×5 mm is collected from the right brain hemisphere (e.g., cerebellum (lateral), cerebellum (dorsal), frontal cortex (Brodmann area 4), frontal cortex (Brodmann area 6), occipital cortex (cortical surface), occipital cortex (parenchyma)), ovary, heart, kidney, lacrimal gland (left), liver (left lateral lobe), lung (left caudal lobe), lymph node (parotid), lymph node (mandibular), pituitary gland, salivary gland (mandibular), spleen, thymus, dorsal root ganglia (cervical, left), dorsal root ganglia (lumbar, left), and dorsal root ganglia (thoracic, left). Samples are stored between −60° C. and −80° C.

Histology: right eye and right optic nerve from animals are sectioned at a nominal 5 μm and stained with hematoxylin and eosin. Eye tissues are sectioned to facilitate examination of the fovea, injection site region, macula, optic disc, and optic nerve. A single, vertical section is taken through the approximate center of the inferior calotte. This results in one slide/block/eye (three slides per eye total). Further, digital scans (virtual slides) can be prepared from selected microscopic slides.

Data Evaluation and Statistical Analysis: statistical data analyses are calculated using means and standard deviations. Means and standard deviations are calculated for absolute body weight, body weight change, and intraocular pressure measurements.

### 5.17 Example 17: Evaluation of a 1% Carboxymethylcellulose High Viscosity Grade Formulation Injectability and Thermal Stability

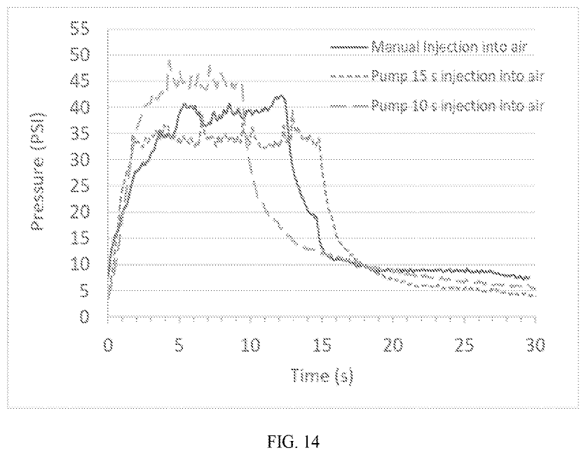

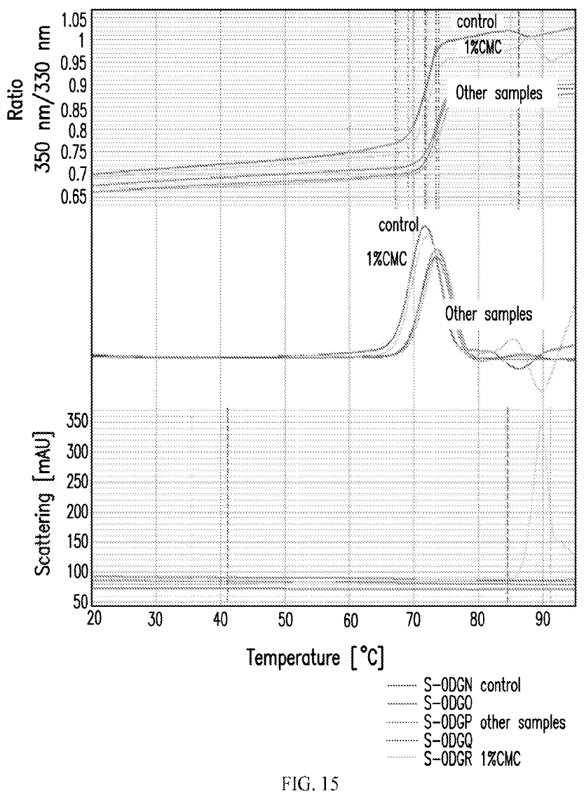

A formulation containing 1% carboxymethylcelluose high viscosity grade prepared in a ‘base’ solution also containing 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL (4% w/v) sucrose, and 0.001% (0.01 mg/mL) poloxamer 188, pH 7.4 was evaluated for stability and injectability. FIG. 14. shows the injection pressure versus time for a manual injection over about 13 s and two injections using a controlled rate syringe pump over 10 s and 15 s. Injections were performed using a Clearside syringe device (CLS-HN001) and a 30 gauge (160 μm ID, CLS-MN1100) needle. The results showed that the pressures obtained are all approximately within the preferred (in some cases)<43 PSI range. The max pressure for the manual injection was 42 PSI, the syringe pump injection over 15 s max pressure was 39 PSI and the syringe pump injection over 10 s max pressure was a brief spike to 49 PSI (FIG. 14). The manual injection was perceived as easy to inject and smooth.

Differential scanning fluorimetry (DSF) measures the intrinsic tryptophan and tyrosine emission of proteins as a function of temperature. The local environment of Trp and Tyr residues changes as the protein unfolds resulting in a large increase in fluorescence. FIG. shows differential scanning fluorimetry thermal ramp data for a control compared to 1% carboxymethylcellulose formulation. Top panel: raw melting curve signal. Middle panel: derivative of data to identify the peak. Bottom panel: light scattering data to indicate aggregation. The 1% carboxymethylcellose formulation (Tₘ=67.52° C.) had a similar profile to the control (Tₘ=67.10° C.) upon thermal ramping which demonstrates that the AAV is stable in this formulation.

### 5.18 Example 18: Pharmacodynamic, Biodistribution, and Tolerability Study in Cynomolgus Monkeys Using Different Suprachoroidal Formulations

The objective of this study was to evaluate the biodistribution (DNA and mRNA), pharmacodynamics (transgene concentration), and tolerability of a 1% CMC formulation comprising AAV8-anti-VEGF-ab when administered as a single dose via suprachoroidal injection to Cynomolgus monkeys. After dosing, animals were observed postdose for at least 4 weeks.

Others have shown that administration of two injections rather than one into the suprachoroidal space of rats or rabbits was associated with greater spread of the injected formulation around the eye compared to a single injection. Increased spread around the eye was associated with increased delivery of therapeutic to the eye in those studies.

To understand the impact of one versus two injections on transgene expression and biodistribution in these studies, one group was administered single injections of the full dose volume into the superior temporal quadrant. The remaining groups were administered two injections to achieve the same dose volume, one injection into the temporal superior quadrant and one into the inferior nasal quadrant. The group assignment and dose levels were shown in Table 10. The test article was AAV8-anti-VEGF-ab. The control article was a placebo.

TABLE 10
Group Assignment and Dose Levels

| | | | Dose | Dose | | |
|---|---|---|---|---|---|---|
| | Dose Regimen | | Level^{b} | Concentration | No. of | Injection |
| Group^{a} | Left Eye | Right Eye | (gc/eye) | (gc/mL) | animals | Volume |
| 1 | Control | Control | 0 | 0 | 1 | 100 μL/ |
| | Article 2 | Article 2 | | | | eye/dose |
| | | | | | | (two 50 μL |
| | | | | | | injections) |
| 2 | Test | Test | 3 × 10¹¹ | 3 × 10¹² | 4 | 100 μL/ |
| | Article 2 | Article 2 | | | | eye/dose |
| | | | | | | (two 50 μL |
| | | | | | | injections) |
| 3 | Test | Test | 3 × 10¹¹ | 3 × 10¹² | 4 | 100 μL/ |
| | Article 2 | Article 2 | | | | eye/dose |
| | | | | | | (one 100 μL |
| | | | | | | injection) |

gc = genome copies
^{a}Group 1 was administered control article only.
^{b}Dose levels for Groups 1 and 2 were based on a dose volume of 100 μL/eye/dose administered as two 50 μL injections. Dose levels for Group 3 were based on a dose volume of 100 μL/eye administered as one injection of 100 μL/eye/dose.
c. All animals were sacrificed on Day 29 of the dosing phase.

Dose Administration: the preparation of test articles and control articles is shown in Table 11. The test articles and control articles were stored in a freezer between −60° C. and −80° C. and thawed at room temperature on the day of use. The formulations were thawed and stored at room temperature. Animals were not fasted overnight prior to dosing. Animals were anesthetized with ketamine and dexmedetomidine prior to suprachoroidal injection. In administration, two suprachoroidal injections of 50 μL (Groups 1 and 2) or one injection of 100 μL (total of 100 μL/eye; Group 3) was administered to each eye (between 3 and 4 mm from the limbus) over 10 to 15 seconds. The syringe and microneedle size are shown in Table 11. The first injection in the right eye was administered in the superior temporal quadrant (i.e., between the 10 o'clock and 11 o'clock positions), and the second injection in the right eye (as applicable) was administered in the inferior nasal quadrant (i.e., between the 4 o'clock and 5 o'clock positions). The first injection in the left eye was administered in the superior temporal quadrant (i.e., between the 1 o'clock and 2 o'clock positions), and the second injection in the left eye (as applicable) was administered in the inferior nasal quadrant (i.e., between the 7 o'clock and 8 o'clock positions). Following the injection, the needle was kept in the eye for approximately 30 seconds before being withdrawn. Upon withdrawal of the micro needle, a cotton-tipped applicator (dose wipe) was placed over the injection site for approximately 10 seconds.

TABLE 11
Preparation of Test and Vehicle Control Articles

| | Formulation | Composition | Syringe Preparation |
|---|---|---|---|
| Test | 1% | In ready-to-use vials containing | Clearside microinjector syringe |
| Article | Carboxymethyl | 1.1 mL at 3 × 10¹² genome | (CLS-HN001) with an attached |
| 2 | cellulose | copies (GC)/mL in 1% CMC | vial adapter (Medimop Medical |
| | (CMC) | formulation | Projects Ltd, Part No. |
| | formulation | | 8070129), and affixed to a 30- |
| | | | gauge × 700 μm microneedle |
| | | | (Clearside Biomedical, Inc., |
| | | | Part No. CLSD0707 CLS- |
| | | | A700) |
| Control | Placebo 1% | 1.0% carboxymethylcellulose | Clearside microinjector syringe |
| Article | CMC | sodium in modified DPBS with | (CLS-HN001) with an attached |
| 2 | formulation | sucrose (5.84 mg/mL sodium | vial adapter (Medimop Medical |
| | (vehicle) | chloride, 0.201 mg/mL | Projects Ltd, Part No. |
| | | potassium chloride, 1.15 | 8070129), and affixed to a 30- |
| | | mg/mL sodium phosphate | gauge × 700 μm microneedle |
| | | dibasic anhydrous, 0.200 | (Clearside Biomedical, Inc., |
| | | mg/mL potassium phosphate | Part No. CLSD0707 CLS- |
| | | monobasic, 40.0 mg/mL (4% | A700) |
| | | w/v) sucrose, 0.001% (0.01 | |
| | | mg/mL) poloxamer 188, pH | |
| | | 7.4) | |

Ophthalmic Procedures: ophthalmic examinations on days 4, 8, 15, and 29 post administration were conducted. Animals were examined with a slit lamp biomicroscope and indirect ophthalmoscope. The adnexa and anterior portion of both eyes were examined using a slit lamp biomicroscope. The ocular fundus of both eyes were examined (where visible) using an indirect ophthalmoscope. Prior to examination with the indirect ophthalmoscope, pupils were dilated with a mydriatic agent (e.g., 1% tropicamide). Intraocular pressure (TOP) was measured on the day of administration (within 10 minutes prior to dosing) and on days 4, 8, 15, and 29. The IOP measurements were done using an applanation tonometer. Ocular photography was performed during Week 4. Photographs were taken with a digital fundus camera and wide angle lens. Color photographs were taken of each eye to include stereoscopic photographs of the posterior pole and nonstereoscopic photographs of two peripheral fields (superior temporal and inferior nasal).

Anti-AAV8-anti-VEGF-ab Transgene Product Antibody Analysis: blood samples were taken as discussed above once predose, and on the day of scheduled sacrifice (Day 29). Serum samples were analyzed for antibodies to the AAV8-anti-VEGF-ab using a validated antibody assay. For AAV8-anti-VEGF-ab transgene analysis, blood samples were taken as described above at least two weeks prior to administration, on Day 15, and on the day of scheduled sacrifice (Day 29). Samples were then analyzed by a validated antibody assay.

Aqueous Humor Collection: approximately 50 μL was removed from each eye at least 2 weeks prior to administration, on Day 15, and on the day the scheduled sacrificed (Day 29). Aqueous humor samples from each eye were placed into separate tubes with Watson barcoded labels, snap frozen in liquid nitrogen, and placed on dry ice until stored between −60° C. and −80° C. Samples were analyzed for anti-VEGF concentration by a validated method.

Termination of Study: animals were anesthetized with sodium pentobarbital and exsanguinated on Day 29.

Necropsy Collections of Aqueous Humor and Vitreous Humor: Following exsanguination, eyes were enucleated and aqueous humor and vitreous humor samples were collected. Following collection, samples were flash-frozen and stored between −60° C. and −80° C. Aqueous and vitreous samples were analyzed for transgene concentration by a validated method.

Ocular Tissue Collection for Biodistribution: following exsanguination, the right eye from each animal and the left eye from the last two animals in Groups 2 and 3 were enucleated and tissues were collected. Tissues were collected into separate tubes. Collected tissue included choroid with retinal pigmented epithelium, retina, and sclera. Tissues were collected using ultra-clean procedures as described above, and rinsed with saline and blotted dry. Samples were flash-frozen and stored between −60° C. and −80° C. Samples were analyzed for vector DNA or RNA using a qPCR or qRT-PCR method.

Comparator study: in a Cynomolgous monkey study conducted analogously to the protocols described in this Example, a control formulation (control article 2.5) was injected to the SCS of each eye (temporal superior and nasal inferior injection with microinjector). The control formulation does not contain carboxymethylcellulose sodium.

TABLE 13
Preparation of Control Formulation

| | | | Syringe |
|---|---|---|---|
| | Formulation | Composition | Preparation |
| Control | Control SCS | Modified DPBS with sucrose | Clearside |
| Article | formulation | (5.84 mg/mL sodium chloride, | microinjector |
| 2.5 | | 0.201 mg/mL potassium | syringe as |
| | | chloride, 1.15 mg/mL sodium | described in |
| | | phosphate dibasic anhydrous, | Table 11 |
| | | phosphate 0.200 mg/mL potassium | |
| | | monobasic, 40.0 mg/mL (4% w/v) | |
| | | sucrose, 0.001% (0.01 mg/mL) | |
| | | poloxamer 188, pH 7.4) | |

The control formulation also contained AAV8-anti-VEGF-ab and was dosed at 3×10¹¹ gc/eye in 100 μL/eye/dose (two 50 μL injections).

Data Evaluation and Statistical Analysis: statistical data analyses were calculated using means and standard deviations. Transgene product (“TP”, protein) in aqueous humor was assessed at 15 and 29 days, otherwise TP, DNA and RNA was assessed in vitreous humor, serum and liver at 29 days.

### Results

TABLE 14
Aqueous Humor Transgene Product (ng/ml)

| | | | Test | | Test | | Control | |
|---|---|---|---|---|---|---|---|---|
| | Control | | Article 2- | | Article 2- | | Article 2.5- | |
| | Article 2- | | one | | two | | control | |
| | placebo | | injection | | injections | | formulation | |
| | (TP ng/mL) | | (TP ng/mL) | | (TP ng/mL) | | (TP ng/mL) | |
| | 15 | 29 | 15 | 29 | 15 | 29 | 15 | 29 |
| | days | days | days | days | days | days | days | days |
| Avg. | 0^{a} | 0^{a} | 43.7 | 90.9 | 11.67 | 19.26 | 2.79 | 3.69 |

^{a}when values were below limit of quantification (<0.100 ng/mL), a value of “0” was assigned for calculation of descriptive statistics.

One injection of Test article 2 (1% CMC) into the SCS at the temporal superior as well as two injections of Test article 2 (1% CMC) resulted in greater transgene product (TP) concentration in aqueous humor compared to the Control Formulation. One injection of Test article 2 increased TP concentration in the aqueous humor compared to two injections having the same composition however administered at full dose in one injection at the temporal superior location.

TABLE 15
Vitreous Humor Transgene Product (ng/mL)

| | Control | Test Article | Test Article | Control Article |
|---|---|---|---|---|
| | Article | 2- one | 2- two | 2.5- control |
| | 2- placebo | injection | injections | formulation |
| | (TP ng/mL) | (TP ng/mL) | (TP ng/mL) | (TP ng/mL) |
| Avg. | 0^{a} | 200 | 49 | 17.99 |

^{a}when values were below limit of quantification (<0.100 ng/mL), a value of “0” was assigned for calculation of descriptive statistics.

One injection of Test article 2 (1% CMC) into the SCS at the temporal superior location produces the highest concentrations of transgene product in the VH, compared to two injections of Test article 2 (1% CMC) and the Control Formulation. Vitreous humor transgene product concentration was higher overall than TP found in aqueous humor 29 days following injection.

TABLE 16
Serum Transgene Product (ng/mL)

| | Control | Test Article | Test Article | Control Article |
|---|---|---|---|---|
| | Article | 2- one | 2- two | 2.5- control |
| | 2- placebo | injection | injections | formulation |
| | (TP ng/mL) | (TP ng/mL) | (TP ng/mL) | (TP ng/mL) |
| Avg. | 0^{a} | 1.73 | 1.02 | 0.44 |

^{a}when values were below limit of quantification (<0.100 ng/mL), a value of “0” was assigned for calculation of descriptive statistics.

Each injection of Test article 2 (1% CMC) or Control formulation containing AAV8-anti-VEGF-ab into the SCS produced minimal titers of transgene product (anti-VEGF-ab) in the serum.

TABLE 17
DNA or RNA (copies/μg) Biodistribution in Tissues

| | | | Test | | Test | | Control | |
|---|---|---|---|---|---|---|---|---|
| | Control | | Article 2- | | Article 2- | | Article 2.5- | |
| | Article 2- | | one | | two | | control | |
| | placebo | | injection | | injections | | formulation | |
| | (copies/μg) | | (copies/μg) | | (copies/μg) | | (copies/μg) | |
| | DNA | RNA | DNA | RNA | DNA | RNA | DNA | RNA |
| Retina | nt | nt | 1.17E+05 | 3.97E+06 | 1.34E+04 | 5.85E+05 | 5.61E+03 | nt |
| (Avg.) | | | | | | | | |
| RPE/ | nt | nt | 2.78E+07 | 2.46E+06 | 4.01E+07 | 4.85E+05 | 3.97E+06 | nt |
| Choroid | | | | | | | | |
| (Avg.) | | | | | | | | |
| Sclera | nt | nt | 2.65E+08 | 7.85E+05 | 1.82E+08 | 8.82E+05 | 7.92E+07 | nt |
| (Avg.) | | | | | | | | |

nt = not tested

The CMC formulation injected once into the eye appeared to transduce retina more efficiently than administering two injections at the same dose. Overall, Test article 2 (1% CMC) compared to Control formulation increased vector delivery to both the choroid and sclera.

### 5.19 Example 19: Stability and Compatibility of Different Suprachoroidal Formulations

The objective of this study was to evaluate the stability and compatibility with devices for AAV8-anti-VEGF-ab formulated with 1.0% Carboxymethylcellulose Sodium in Modified DPBS with Sucrose (5.84 mg/mL Sodium Chloride, 0.201 mg/mL Potassium Chloride, 1.15 mg/mL Sodium Phosphate Dibasic Anhydrous, 0.200 mg/mL Potassium Phosphate Monobasic, 40.0 mg/mL (4% w/v) Sucrose, 0.001% Poloxamer 188, pH 7.4).

The stability results indicate the formulated AAV8-anti-VEGF-ab test and placebo articles remained stable at the storage temperature of ≤−60° C. for the duration of the study. The end of study testing was initiated 3.3 months after filling.

The in-use stability results indicate that formulated AAV8-anti-VEGF-ab is stable for up to 6 hours at room temperature. In addition, it was demonstrated that formulated AAV8-anti-VEGF-ab is compatible with the suprachoroidal delivery system intended for use in this study. Supporting data are provided in the table below.

TABLE 18
In-Use Stability and Device Compatibility

| | | | | In-Use |
|---|---|---|---|---|
| | | | In-Use | Stability |
| | | Control | Stability | in Device |
| | Analytical | (Result at | in Device | (RT, T = |
| Test | Method | Release) | (T0) | 6 hr) |
| Vector | Transgene | 3.07 × 10¹² | 3.11 × 10¹² | 3.93 × 10¹² |
| Genome | ddPCR | GC/mL^{a} | GC/mL^{a} | GC/mL^{a} |
| Concen- | | | | |
| tration | | | | |
| (Content) | | | | |
| In Vitro | HEK293 | 90% | 120% | 115% |
| Potency | Transduction/ | | | |
| (Potency) | Anti-VEGF | | | |
| | ELISA | | | |

^{a}Sample was diluted 10X to avoid matrix interference. The result was corrected for testing dilution.
RT = room temperature

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed is:

1. A pharmaceutical composition suitable for administration to the suprachoroidal space (SCS) of an eye of a human subject, wherein the pharmaceutical composition comprises a recombinant adeno-associated virus (AAV) vector comprising an expression cassette encoding a transgene, and wherein the pharmaceutical composition has viscosity of between about 25 cP to about 3×10⁶ cP as measured at a shear rate of at most about 1 s⁻¹.

2. A pharmaceutical composition suitable for administration to the suprachoroidal space (SCS) of an eye of a human subject, wherein the pharmaceutical composition comprises a recombinant adeno-associated virus (AAV) vector comprising an expression cassette encoding a transgene, wherein the transgene is an anti-human vascular endothelial growth factor (anti-VEGF) antibody, and wherein the pharmaceutical composition has viscosity of between about 25 cP to about 3×10⁶ cP as measured at a shear rate of at most about 1 s⁻¹.

3. The pharmaceutical composition of claim 1 or 2, wherein the clearance time after suprachoroidal administration is equal to or greater than the clearance time of a reference pharmaceutical composition after suprachoroidal administration, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹.

4. The pharmaceutical composition of claim 1 or 2, wherein a circumferential spread after suprachoroidal administration is smaller as compared to a circumferential spread of a reference pharmaceutical composition after suprachoroidal administration, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹.

5. The pharmaceutical composition of claim 1 or 2, wherein a thickness at a site of injection after suprachoroidal administration is equal to or higher as compared to a thickness at a site of injection after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹.

6. The pharmaceutical composition of claim 1 or 2, wherein an expression level of the transgene is detected in the eye for a longer period of time after suprachoroidal administration as compared to a period of time that an expression level of the transgene is detected in the eye after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹.

7. The pharmaceutical composition of claim 1 or 2, wherein the concentration of the transgene in the eye after suprachoroidal administration is equal to or higher as compared to the concentration of the transgene in the eye after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹.

8. The pharmaceutical composition of claim 1 or 2, wherein the rate of transduction at a site of injection after suprachoroidal administration is equal to or higher as compared to the rate of transduction at a site of injection after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹.

9. The pharmaceutical composition of claim 2, wherein a level of VEGF-induced vasodilation and/or vascular leakage after suprachoroidal administration is equal to or decreased as compared to a level of VEGF-induced vasodilation and/or vascular leakage after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the reference pharmaceutical composition has a viscosity of at most about 24 cP as measured at a shear rate of at most about 1 s⁻¹.

10. The pharmaceutical composition of any one of claims 1-9, wherein the recombinant AAV is Construct II.

11. The pharmaceutical composition of any one of claims 1, 3-8 and 10, wherein the transgene is an anti-human vascular endothelial growth factor (anti-VEGF) antibody.

12. The pharmaceutical composition of any one of claims 1-11, wherein the recombinant AAV comprises components from one or more adeno-associated virus serotypes selected from the group consisting of AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

13. The pharmaceutical composition of any one of claims 1-12, wherein the recombinant AAV is AAV8.

14. The pharmaceutical composition of any one of claims 1-9 and 11-12, wherein the recombinant AAV is AAV9.

15. The pharmaceutical composition of any one of claims 1-14, wherein the pharmaceutical composition has a viscosity of between about 25 cP to about 100,000 cP, between about 25 cP to about 50,000 cP, between about 25 cP to about 1×10⁴ cP, between about cP to about 5,000 cP, between about 25 cP to about 1×10³ cP, between about 100 cP to about 100,000 cP, between about 100 cP to about 1×10⁴ cP, between about 100 cP to about 5,000 cP, between about 100 cP to about 1×10³ cP, as measured at a shear rate of at most about 1 s⁻¹.

16. The pharmaceutical composition of any one of claims 1-15, wherein the pharmaceutical composition has viscosity of at least about 100 cP, at least about 400 cP, at least about 500 cP, at least about 900 cP, at least about 1000 cP, at least about 4000 cP, or at least about 1×10⁶ cP, as measured at a shear rate of at most about 1 s⁻¹.

17. The pharmaceutical composition of any one of claims 1-16, wherein the pharmaceutical composition has viscosity of about or greater than about 500 cP as measured at a shear rate of at most about 1 s⁻¹.

18. The pharmaceutical composition of any one of claims 1-17, wherein the pharmaceutical composition comprises sucrose.

19. The pharmaceutical composition of any one of claims 1-17, wherein the pharmaceutical composition does not comprise sucrose.

20. The pharmaceutical composition of any one of claims 1-19, wherein the pharmaceutical composition comprises at least one of sucrose, 4% sucrose, 6% sucrose, 10% sucrose, 2% carboxymethyl cellulose sodium salt, 1% carboxymethyl cellulose sodium salt, carboxymethyl cellulose (CMC), 0.5% CMC, 1% CMC, 2% CMC, 4% CMC, polyvinyl alcohol, hydroxyethyl cellulose, carboxymethyl cellulose sodium salt, and hydroxypropyl methylcellulose.

21. The pharmaceutical composition of any one of claims 1-18 and 20, wherein the pharmaceutical composition comprises 4% sucrose, 6% sucrose, or 10% sucrose.

22. The pharmaceutical composition of any one of claims 4 and 10-21, wherein the circumferential spread after suprachoroidal administration of the pharmaceutical composition is smaller by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

23. The pharmaceutical composition of any one of claims 3 and 10-22, wherein the clearance time after suprachoroidal administration of the pharmaceutical composition is greater by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or at least 500%.

24. The pharmaceutical composition of any one of claims 1-23, wherein the clearance time after suprachoroidal administration of the pharmaceutical composition is of about 30 minutes to about 20 hours, about 2 hours to about 20 hours, about 30 minutes to about 24 hours, about 1 hour to about 2 hours, about 30 minutes to about 90 days, about 30 minutes to about 60 days, about 30 minutes to about 30 days, about 30 minutes to about 21 days, about 30 minutes to about 14 days, about 30 minutes to about 7 days, about 30 minutes to about 3 days, about 30 minutes to about 2 days, about 30 minutes to about 1 day, about 4 hours to about 90 days, about 4 hours to about 60 days, about 4 hours to about 30 days, about 4 hours to about 21 days, about 4 hours to about 14 days, about 4 hours to about 7 days, about 4 hours to about 3 days, about 4 hours to about 2 days, about 4 hours to about 1 day, about 4 hours to about 8 hours, about 4 hours to about 16 hours, about 4 hours to about 20 hours, about 1 day to about 90 days, about 1 day to about 60 days, about 1 day to about 30 days, about 1 day to about 21 days, about 1 day to about 14 days, about 1 day to about 7 days, about 1 day to about 3 days, about 2 days to about 90 days, about 3 days to about 90 days, about 3 days to about 60 days, about 3 days to about 30 days, about 3 days to about 21 days, about 3 days to about 14 days, or about 3 days to about 7 days.

25. The pharmaceutical composition of any one of claims 1-24, wherein the clearance time after suprachoroidal administration of the pharmaceutical composition is not prior to about minutes, 1 hour, 2 hours, 3 hours, 4 hours, 5 hours, 6 hours, 7 hours, 8 hours, 9 hours, hours, 12 hours, 14 hours, 16 hours, 18 hours, 20 hours, 22 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, days, 100 days, 120 days, 140 days, 160 days, 180 days, 200 days, 220 days, 240 days, 260 days, 280 days, 300 days, 320 days, 340 days, 360 days, 380 days, or 400 days.

26. The pharmaceutical composition of any one of claims 3-25, wherein the clearance time of the reference pharmaceutical composition after suprachoroidal administration is of at most about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days, 3 days, 4 days, 5 days, 6 days, 7 days, 8 days, 9 days, 10 days, 11 days, 12 days, 13 days, or 14 days.

27. The pharmaceutical composition of any one of claims 1-26, wherein the clearance time is from the SCS or from the eye.

28. The pharmaceutical composition of any one of claims 5 and 10-27, wherein the thickness at the site of injection after suprachoroidal administration of the pharmaceutical composition is higher by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

29. The pharmaceutical composition of any one of claims 5 and 10-28, wherein the thickness at the site of injection after suprachoroidal administration of the pharmaceutical composition is about 500 μm to about 3.0 mm, 750 μm to about 2.8 mm, about 750 μm to about 2.5 mm, about 750 μm to about 2 mm, or about 1 mm to about 2 mm.

30. The pharmaceutical composition of any one of claims 5 and 10-29, wherein the thickness at the site of injection after suprachoroidal administration of the pharmaceutical composition is of at least about 50 μm, 100 μm, 200 μm, 300 μm, 400 μm, 500 μm, 600 μm, 700 μm, 800 μm, 900 μm, 1000 μm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, or 10 mm.

31. The pharmaceutical composition of any one of claims 5 and 10-30, wherein the thickness at the site of injection after suprachoroidal administration of the reference pharmaceutical composition is of at most about 1 nm, 5 nm, 10 nm, 25 nm, 50 nm, 100 nm, 200 nm, 300 nm, 400 nm, 500 nm, 600 nm, 700 nm, 800 nm, 900 nm, 1 μm, 5 μm, 10 μm, 15 μm, 20 μm, 25 μm, 30 μm, 35 μm, 40 μm, 50 μm, 100 μm, 200 μm, 300 μm, 400 μm, 500 μm, 600 μm, 700 μm, 800 μm, 900 μm, or 1000 μm.

32. The pharmaceutical composition of any one of claims 5 and 10-31, wherein the thickness at the site of injection after suprachoroidal administration of the pharmaceutical composition persists for at least two hours, at least three hours, at least four hours, at least five hours, at least six hours, at least seven hours, at least eight hours, at least ten hours, at least twelve hours, at least eighteen hours, at least twenty-four hours, at least two days, at least three days, at least five days, at least ten days, at least twenty-one days, at least one month, at least six weeks, at least two months, at least three months, at least 4 months, at least 5 months, at least 6 months, at least 9 months, at least one year, at least three years, or at least five years.

33. The pharmaceutical composition of any one of claims 7 and 10-32, wherein the concentration of the transgene in the eye after suprachoroidal administration of the pharmaceutical composition is higher by at least 2 times, at least 3 times, at least 4 times, at least times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

34. The pharmaceutical composition of any one of claims 6 and 10-33, wherein the longer period of time after suprachoroidal administration of the pharmaceutical composition is longer by at least 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

35. The pharmaceutical composition of any one of claims 1-34, wherein the transgene is detected in the eye after suprachoroidal administration of the pharmaceutical composition for at least about 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

36. The pharmaceutical composition of any one of claims 3-35, wherein the transgene is detected in the eye after suprachoroidal administration of the reference pharmaceutical composition for at most about 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, days, 21 days, 23 days, 25 days, 27 days, 30 days, 35 days, 40 days, 50 days, 55 days, 60 days, 65 days, 70 days, 75 days, 80 days, 85 days, 90 days, 95 days, or 100 days, 120 days, 140 days, 160 days, 180 days, 200 days, 220 days, 240 days, 260 days, 280 days, 300 days, 320 days, 340 days, 360 days, 380 days, or 400 days after.

37. The pharmaceutical composition of claim 11, wherein a level of VEGF-induced vasodilation and/or vascular leakage after suprachoroidal administration of the pharmaceutical composition is equal to or decreased as compared to a level of VEGF-induced vasodilation and/or vascular leakage after suprachoroidal administration of the reference pharmaceutical composition.

38. The pharmaceutical composition of any one of claims 9-37, wherein the level of VEGF-induced vasodilation and/or vascular leakage after suprachoroidal administration of the pharmaceutical composition is decreased by at least about 2 times, at least 3 times, at least 4 times, at least 5 times, at least 6 times, at least 7 times, at least 8 times, at least 9 times, at least times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

39. The pharmaceutical composition of any one of claims 8 and 10-38, wherein the rate of transduction at the site of injection after suprachoroidal administration of the pharmaceutical composition is higher by at least about 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

40. The pharmaceutical composition of any one of claims 1-39, wherein the recombinant AAV stability in the pharmaceutical composition is at least about 50% the recombinant AAV stability in the reference pharmaceutical composition.

41. The pharmaceutical composition of claim 40, wherein the recombinant AAV stability is determined by infectivity of the recombinant AAV.

42. The pharmaceutical composition of claim 40, wherein the recombinant AAV stability is determined by a level of aggregation of the recombinant AAV.

43. The pharmaceutical composition of claim 40, wherein the recombinant AAV stability is determined by a level of free DNA released by the recombinant AAV.

44. The pharmaceutical composition of claim 43, wherein the pharmaceutical composition comprises about 50% more, about 25% more, about 15% more, about 10% more, about 5% more, about 4% more, about 3% more, about 2% more, about 1% more, about 0% more, about 1% less, about 2% less, about 5% less, about 7% less, about 10% less, about 2 times more, about 3 times more, about 2 times less, about 3 times less, free DNA as compared to a level of free DNA in the reference pharmaceutical composition.

45. The pharmaceutical composition of claim 41, wherein the recombinant AAV in the pharmaceutical composition has an infectivity that is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times higher as compared to the infectivity of the recombinant AAV in the reference pharmaceutical composition.

46. The pharmaceutical composition of claim 42, wherein the pharmaceutical composition comprises at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times less recombinant AAV aggregation as compared to a level of the recombinant AAV aggregation in the reference pharmaceutical composition.

47. The pharmaceutical composition of any one of claims 1-46, wherein the transgene is a transgene suitable to treat, or otherwise ameliorate, prevent or slow the progression of a disease of interest.

48. The pharmaceutical composition of any one of claims 1-47, wherein the human subject is diagnosed with nAMD (wet AMD), dry AMD, retinal vein occlusion (RVO), diabetic macular edema (DME), diabetic retinopathy (DR), Batten disease, glaucoma, or non-infectious uveitis.

49. The pharmaceutical composition of any one of claims 1-47, wherein the human subject is diagnosed with mucopolysaccharidosis type IVA (MPS IVA), mucopolysaccharidosis type I (MPS I), mucopolysaccharidosis type II (MPS II), familial hypercholesterolemia (FH), homozygous familial hypercholesterolemia (HoFH), coronary artery disease, cerebrovascular disease, Duchenne muscular dystrophy, Limb Girdle muscular dystrophy, Becker muscular dystrophy and sporadic inclusion body myositis, or kallikrein-related disease.

50. The pharmaceutical composition of any one of claims 1, 3-8 and 10-49, wherein the AAV encodes Palmitoyl-Protein Thioesterase 1 (PPT1), Tripeptidyl-Peptidase 1 (TPP1), anti-VEGF antibody or antigen-binding fragment thereof, anti-kallikrein antibody or antigen-binding fragment, anti-TNF antibody or antigen-binding fragment, anti-C3 antibody or antigen-binding fragment, or anti-05 antibody or antigen-binding fragment.

51. The pharmaceutical composition of any one of claims 3-50, wherein the amount of the recombinant AAV genome copies is based on a vector genome concentration.

52. The pharmaceutical composition of any one of claims 3-50, wherein the amount of the recombinant AAV genome copies is based on genome copies per administration.

53. The pharmaceutical composition of any one of claims 3-50, wherein the amount of the recombinant AAV genome copies is based on total genome copies administered to the human subject.

54. The pharmaceutical composition of claim 52, wherein the genome copies per administration is the genome copies of the recombinant AAV per suprachoroidal administration.

55. The pharmaceutical composition of claim 53, wherein the total genome copies administered is the total genome copies of the recombinant AAV administered suprachoroidally.

56. The pharmaceutical composition of claim 51, wherein the vector genome concentration (VGC) is of about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 2×10¹¹ GC/mL, about 2.4×10¹¹ GC/mL, about 2.5×10¹¹ GC/mL, about 3×10¹¹ GC/mL, about 6.2×10¹¹ GC/mL, about 1×10¹² GC/mL, about 2.5×10¹² GC/mL, about 3×10¹² GC/mL, about 5×10¹² GC/mL, about 6×10¹² GC/mL, about 1.5×10¹³ GC/mL, about 2×10¹³ GC/mL, or about 3×10¹³ GC/mL.

57. The pharmaceutical composition of any one of claims 53 and 55, wherein the total number of genome copies administered is about 6.0×10¹⁰ genome copies, about 1.6×10¹¹ genome copies, about 2.5×10¹¹ genome copies, about 3×10¹¹ genome copies, about 5.0×10¹¹ genome copies, about 6×10¹¹ genome copies, about 3×10¹² genome copies, about 1.0×10¹² genome copies, about 1.5×10¹² genome copies, about 2.5×10¹² genome copies, or about 3.0×10¹³ genome copies.

58. The pharmaceutical composition of any one of claims 52 and 54, wherein the total number of genome copies per administration is about 6.0×10¹⁰ genome copies, about 1.6×10¹¹ genome copies, about 2.5×10¹¹ genome copies, about 3×10¹¹ genome copies, about 5.0×10¹¹ genome copies, about 3×10¹² genome copies, about 1.0×10¹² genome copies, about 1.5×10¹² genome copies, about 2.5×10¹² genome copies, or about 3.0×10¹³ genome copies.

59. The pharmaceutical composition of any one of claims 1-58, wherein the pharmaceutical composition is administered once, twice, three times, four times, five times, six times, seven times, eight times, nine times, ten times, fifteen times, twenty times, twenty five times, or thirty times.

60. The pharmaceutical composition of any one of claims 3-59, wherein the reference pharmaceutical composition is administered once, twice, three times, four times, five times, six times, seven times, eight times, nine times, ten times, fifteen times, twenty times, twenty five times, or thirty times.

61. The pharmaceutical composition of any one of claims 1-60, wherein the pharmaceutical composition is administered once in one day, twice in one day, three times in one day, four times in one day, five times in one day, six times in one day, or seven times in one day.

62. The pharmaceutical composition of any one of claims 3-60, wherein the reference pharmaceutical composition is administered once in one day, twice in one day, three times in one day, four times in one day, five times in one day, six times in one day, or seven times in one day.

63. The pharmaceutical composition of any one of claims 1-60, wherein the reference pharmaceutical composition comprises DPBS and sucrose.

64. The pharmaceutical composition of any one of claims 3-60, wherein the reference pharmaceutical composition has a viscosity of about 1 cP as measured at a shear rate of at most about 1 s⁻¹.

65. The pharmaceutical composition of any one of claims 1-64, wherein the pharmaceutical composition comprises 0.2 to 15% carboxymethyl cellulose (CMC) high viscosity grade, CMC high viscosity grade, CMC medium viscosity grade, hydroxypropyl methylcellulose (HPMC), HPMC, hydroxyethyl cellulose (HES), CMC low viscosity grade, and/or poloxamer 407.

66. The pharmaceutical composition of any one of claims 1-65, wherein the viscosity of the pharmaceutical composition is measured at a shear rate of 0 s⁻¹.

67. The pharmaceutical composition of any one of claims 3-66, wherein the viscosity of the reference pharmaceutical composition is measured at a shear rate of 0 s⁻¹.

68. The pharmaceutical composition of any one of claims 1-67, wherein the viscosity of the pharmaceutical composition and the viscosity of the reference pharmaceutical composition is measured at the same shear rate.

69. The pharmaceutical composition of any one of claims 1-68, wherein the viscosity of the pharmaceutical composition is shear-thinning.

70. The pharmaceutical composition of any one of claims 1-69, wherein the viscosity of the pharmaceutical composition is about, at most about, or at least about 0.1 cP, 0.2 cP, 0.3 cP, cP, 0.5 cP, 0.6 cP, 0.7 cP, 0.8 cP, 0.9 cP, 1 cP, 2 cP, 3 cP, 4 cP, 5 cP, 10 cP, 20 cP, 25 cP, 30 cP, 35 cP, 40 cP, 50 cP, 60 cP, 70 cP, 80 cP, 90 cP, 100 cP, 150 cP, 200 cP, 250 cP, 300 cP, 350 cP, 400 cP, 450 cP, 500 cP, 550 cP, 600 cP, 650 cP, 700 cP, 750 cP, 800 cP, 850 cP, 900 cP, 950 cP, 1000 cP, 1500 cP, 2000 cP, 2500 cP, 3000 cP, 3500 cP, 4000 cP, 4500 cP, 5000 cP, or 10000 cP, as measured at a shear rate of at least about 1,000 s⁻¹.

71. The pharmaceutical composition of claim 70, wherein the viscosity of the pharmaceutical composition is measured at a shear rate of at least about 1,000 s⁻¹, 2,000 s⁻¹, 3,000 s⁻¹, 4,000 s⁻¹, 5,000 s⁻¹, 6,000 s⁻¹, 7,000 s⁻¹, 8,000 s⁻¹, 9,000 s⁻¹, 10,000 s⁻¹, 15,000 s⁻¹, s⁻¹, or 30,000 s⁻¹.

72. The pharmaceutical composition of any one of claims 1-71, wherein the viscosity of the pharmaceutical composition is about or at most about 35 cP as measured at a shear rate of about 5,000 s⁻¹.

73. The pharmaceutical composition of any one of claims 1-71, wherein the viscosity of the pharmaceutical composition is about or at most about 25 cP as measured at a shear rate of about 10,000 s⁻¹.

74. The pharmaceutical composition of any one of claims 1-71, wherein the viscosity of the pharmaceutical composition is about or at least about 500 cP as measured at a shear rate of at most about 1 s⁻¹.

75. The pharmaceutical composition of any one of claims 1-71, wherein the viscosity of the pharmaceutical composition is about or at least about 1500 cP as measured at a shear rate of at most about 1 s⁻¹.

76. The pharmaceutical composition of any one of claims 1-71, wherein the viscosity of the pharmaceutical composition is about or at most about 362 cP as measured at a shear rate of at least about 1000 s⁻¹.

77. The pharmaceutical composition of any one of claims 3-76, wherein the viscosity of the reference pharmaceutical composition is about or at most about 0.1 cP, 0.2 cP, 0.3 cP, 0.4 cP, 0.5 cP, 0.6 cP, 0.7 cP, 0.8 cP, 0.9 cP, 1 cP, 1.1 cP, 1.2 cP, 1.3 cP, 1.4 cP, 1.5 cP, 1.6 cP, 1.7 cP, 1.8 cP, 1.9 cP, 2 cP, 2.1 cP, 2.2 cP, 2.3 cP, 2.4 cP, 2.5 cP, 2.6 cP, 2.7 cP, 2.8 cP, 2.9 cP, 3 cP, 3.1 cP, 3.2 cP, 3.3 cP, 3.4 cP, 3.5 cP, 3.6 cP, 3.7 cP, 3.8 cP, 3.9 cP, 4 cP, 4.1 cP, 4.2 cP, 4.3 cP, 4.4 cP, 4.5 cP, 4.6 cP, 4.7 cP, 4.8 cP, 4.9 cP, or 5 cP as measured at a shear rate of at least about 1000 s⁻¹.

78. The pharmaceutical composition of any one of claims 3-77, wherein the viscosity of the reference pharmaceutical composition is about or at most about 0.1 cP, 0.2 cP, 0.3 cP, 0.4 cP, 0.5 cP, 0.6 cP, 0.7 cP, 0.8 cP, 0.9 cP, 1 cP, 1.1 cP, 1.2 cP, 1.3 cP, 1.4 cP, 1.5 cP, 1.6 cP, 1.7 cP, 1.8 cP, 1.9 cP, 2 cP, 2.1 cP, 2.2 cP, 2.3 cP, 2.4 cP, 2.5 cP, 2.6 cP, 2.7 cP, 2.8 cP, 2.9 cP, 3 cP, 3.1 cP, 3.2 cP, 3.3 cP, 3.4 cP, 3.5 cP, 3.6 cP, 3.7 cP, 3.8 cP, 3.9 cP, 4 cP, 4.1 cP, 4.2 cP, 4.3 cP, 4.4 cP, 4.5 cP, 4.6 cP, 4.7 cP, 4.8 cP, 4.9 cP, or 5 cP as measured at a shear rate of at most about 1 s⁻¹.

79. The pharmaceutical composition of any one of claims 1-78, wherein the viscosity of the pharmaceutical composition is about 0.5 cP to about 400 cP as measured at a shear rate of at least about 1000 s⁻¹.

80. The pharmaceutical composition of any one of claims 1-79, wherein the pharmaceutical composition comprises modified Dulbecco's phosphate-buffered saline solution, and optionally a surfactant.

81. The pharmaceutical composition of any one of claims 1-80, wherein the pharmaceutical composition comprises potassium chloride, potassium phosphate monobasic, sodium chloride, sodium phosphate dibasic anyhydrous, sucrose, and optionally one or more surfactants.

82. The pharmaceutical composition of any one of claims 1-81, wherein the pharmaceutical composition comprises 0.2 mg/mL potassium chloride, 0.2 mg/mL potassium phosphate monobasic, 5.84 mg/mL sodium chloride, 1.15 mg/mL sodium phosphate dibasic anyhydrous, 40.0 mg/mL (4% w/v) sucrose, and optionally a surfactant.

83. The pharmaceutical composition of any one of claims 1-82, wherein the pharmaceutical composition comprises potassium chloride, potassium phosphate monobasic, sodium chloride, sodium phosphate dibasic anyhydrous, sucrose, one or more surfactants selected from poloxamer 188, polysorbate 20, and polysorbate 80, and one or more viscosity modifiers selected from CMC high viscosity grade, CMC medium viscosity grade, CMC low viscosity grade, hydroxypropyl methylcellulose (HPMC), hydroxyethyl cellulose (HES), and poloxamer 407.

84. The pharmaceutical composition of any one of claims 1-83, wherein the pharmaceutical composition comprises potassium chloride, potassium phosphate monobasic, sodium chloride, sodium phosphate dibasic anyhydrous, sucrose, optionally one or more surfactants selected from poloxamer 188, polysorbate 20, and polysorbate 80, and optionally one or more viscosity modifiers selected from 0.5% CMC high viscosity grade, 1% CMC high viscosity grade, 0.5% CMC medium viscosity grade, CMC low viscosity grade, 0.5% hydroxypropyl methylcellulose (HPMC), 0.2% HPMC, 2% hydroxyethyl cellulose (HES), and 15% poloxamer 407.

85. The pharmaceutical composition of any one of claims 1-84, wherein the pharmaceutical composition comprises potassium chloride, potassium phosphate monobasic, sodium chloride, sodium phosphate dibasic anyhydrous, sucrose, one or more surfactants selected from poloxamer 188, polysorbate 20, and polysorbate 80, and one or more polysaccharides selected from CMC, HPMC, and HES.

86. The pharmaceutical composition of any one of claims 1-85, wherein the pharmaceutical composition comprises 0.2 mg/mL potassium chloride, 0.2 mg/mL potassium phosphate monobasic, 5.84 mg/mL sodium chloride, 1.15 mg/mL sodium phosphate dibasic anyhydrous, 40.0 mg/mL (4% w/v) sucrose, 0.001% (0.01 mg/mL) poloxamer 188 and 1% carboxymethyl cellulose (CMC) high viscosity grade.

87. The pharmaceutical composition of any one of claims 1-86, wherein the pharmaceutical composition is stored at about room temperature, 20° C., 4° C., or −80° C.

88. The pharmaceutical composition of any one of claims 1-87, wherein the pharmaceutical composition is stored prior to administration to a human subject.


SEQUENCE LISTING (55 sequences)

SEQ ID NO: 1 - protein, 214 aa, Artificial Sequence
  Ranibizumab Fab Amino Acid Sequence - light chain
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 231 aa, Artificial Sequence
  Ranibizumab Fab Amino Acid Sequence - heavy chain
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 3 - protein, 214 aa, Artificial Sequence
  Bevacizumab Fab Amino Acid Sequence - Light chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 231 aa, Artificial Sequence
  Bevacizumab Fab Amino Acid Sequence - Heavy chain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 5 - protein, 26 aa, Artificial Sequence
  VEGF-A signal peptide
MNFLLSWVHWSLALLLYLHHAKWSQA

SEQ ID NO: 6 - protein, 29 aa, Artificial Sequence
  Fibulin-1 signal peptide
MERAAPSRRVPLPLLLLGGLALLAAGVDA

SEQ ID NO: 7 - protein, 19 aa, Artificial Sequence
  Vitronectin signal peptide
MAPLRPLLILALLAWVALA

SEQ ID NO: 8 - protein, 18 aa, Artificial Sequence
  Complement Factor H signal peptide
MRLLAKIICLMLWAICVA

SEQ ID NO: 9 - protein, 19 aa, Artificial Sequence
  Opticin signal peptide
MRLLAFLSLLALVLQETGT

SEQ ID NO: 10 - DNA, 728 bp, Artificial Sequence
  Bevacizumab cDNA - Light chain
gctagcgccaccatgggctggtcctgcatcatcctgttcctggtggccaccgccaccggcgtgcactccgacatccagatgacccagtccccctcctccctgtccgcctccgtgggcgaccgggtgaccatcacctgctccgcctcccaggacatctccaactacctgaactggtaccagcagaagcccggcaaggcccccaaggtgctgatctacttcacctcctccctgcactccggcgtgccctcccggttctccggctccggctccggcaccgacttcaccctgaccatctcctccctgcagcccgaggacttcgccacctactactgccagcagtactccaccgtgccctggaccttcggccagggcaccaaggtggagatcaagcggaccgtggccgccccctccgtgttcatcttccccccctccgacgagcagctgaagtccggcaccgcctccgtggtgtgcctgctgaacaacttctacccccgggaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaaccggggcgagtgctgagcggccgcctcgag

SEQ ID NO: 11 - DNA, 1440 bp, Artificial Sequence
  Bevacizumab cDNA - heavy chain
gctagcgccaccatgggctggtcctgcatcatcctgttcctggtggccaccgccaccggcgtgcactccgaggtgcagctggtggagtccggcggcggcctggtgcagcccggcggctccctgcggctgtcctgcgccgcctccggctacaccttcaccaactacggcatgaactgggtgcggcaggcccccggcaagggcctggagtgggtgggctggatcaacacctacaccggcgagcccacctacgccgccgacttcaagcggcggttcaccttctccctggacacctccaagtccaccgcctacctgcagatgaactccctgcgggccgaggacaccgccgtgtactactgcgccaagtacccccactactacggctcctcccactggtacttcgacgtgtggggccagggcaccctggtgaccgtgtcctccgcctccaccaagggcccctccgtgttccccctggccccctcctccaagtccacctccggcggcaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtggtgaccgtgccctcctcctccctgggcacccagacctacatctgcaacgtgaaccacaagccctccaacaccaaggtggacaagaaggtggagcccaagtcctgcgacaagacccacacctgccccccctgccccgcccccgagctgctgggcggcccctccgtgttcctgttcccccccaagcccaaggacaccctgatgatctcccggacccccgaggtgacctgcgtggtggtggacgtgtcccacgaggaccccgaggtgaagttcaactggtacgtggacggcgtggaggtgcacaacgccaagaccaagccccgggaggagcagtacaactccacctaccgggtggtgtccgtgctgaccgtgctgcaccaggactggctgaacggcaaggagtacaagtgcaaggtgtccaacaaggccctgcccgcccccatcgagaagaccatctccaaggccaagggccagccccgggagccccaggtgtacaccctgcccccctcccgggaggagatgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgtcccccggcaagtgagcggccgcc

SEQ ID NO: 12 - DNA, 733 bp, Artificial Sequence
  Ranibizumab cDNA (Light chain comprising a signal sequence)
gagctccatggagtttttcaaaaagacggcacttgccgcactggttatgggttttagtggtgcagcattggccgatatccagctgacccagagcccgagcagcctgagcgcaagcgttggtgatcgtgttaccattacctgtagcgcaagccaggatattagcaattatctgaattggtatcagcagaaaccgggtaaagcaccgaaagttctgatttattttaccagcagcctgcatagcggtgttccgagccgttttagcggtagcggtagtggcaccgattttaccctgaccattagcagcctgcagccggaagattttgcaacctattattgtcagcagtatagcaccgttccgtggacctttggtcagggcaccaaagttgaaattaaacgtaccgttgcagcaccgagcgtttttatttttccgcctagtgatgaacagctgaaaagcggcaccgcaagcgttgtttgtctgctgaataatttttatccgcgtgaagcaaaagtgcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagttttaatcgtggcgaatgctaatagaagcttggtacc

SEQ ID NO: 13 - DNA, 779 bp, Artificial Sequence
  Ranibizumab cDNA (Heavy chain comprising a signal sequence)
gagctcatatgaaatacctgctgccgaccgctgctgctggtctgctgctcctcgctgcccagccggcgatggccgaagttcagctggttgaaagcggtggtggtctggttcagcctggtggtagcctgcgtctgagctgtgcagcaagcggttatgattttacccattatggtatgaattgggttcgtcaggcaccgggtaaaggtctggaatgggttggttggattaatacctataccggtgaaccgacctatgcagcagattttaaacgtcgttttacctttagcctggataccagcaaaagcaccgcatatctgcagatgaatagcctgcgtgcagaagataccgcagtttattattgtgccaaatatccgtattactatggcaccagccactggtatttcgatgtttggggtcagggcaccctggttaccgttagcagcgcaagcaccaaaggtccgagcgtttttccgctggcaccgagcagcaaaagtaccagcggtggcacagcagcactgggttgtctggttaaagattattttccggaaccggttaccgtgagctggaatagcggtgcactgaccagcggtgttcatacctttccggcagttctgcagagcagcggtctgtatagcctgagcagcgttgttaccgttccgagcagcagcctgggcacccagacctatatttgtaatgttaatcataaaccgagcaataccaaagtggataaaaaagttgagccgaaaagctgcgataaaacccatctgtaatagggtacc

SEQ ID NO: 14 - protein, 11 aa, Artificial Sequence
  Bevacizumab and Ranibizumab Light Chain CDR1
SASQDISNYLN

SEQ ID NO: 15 - protein, 7 aa, Artificial Sequence
  Bevacizumab and Ranibizumab Light Chain CDR2
FTSSLHS

SEQ ID NO: 16 - protein, 9 aa, Artificial Sequence
  Bevacizumab and Ranibizumab Light Chain CDR3
QQYSTVPWT

SEQ ID NO: 17 - protein, 10 aa, Artificial Sequence
  Bevacizumab Heavy Chain CDR1
GYTFTNYGMN

SEQ ID NO: 18 - protein, 17 aa, Artificial Sequence
  Bevacizumab Heavy Chain CDR2
WINTYTGEPTYAADFKR

SEQ ID NO: 19 - protein, 14 aa, Artificial Sequence
  Bevacizumab Heavy Chain CDR3
YPHYYGSSHWYFDV

SEQ ID NO: 20 - protein, 10 aa, Artificial Sequence
  Ranibizumab Heavy Chain CDR1
GYDFTHYGMN

SEQ ID NO: 21 - protein, 14 aa, Artificial Sequence
  Ranibizumab Heavy Chain CDR3
YPYYYGTSHWYFDV

SEQ ID NO: 22 - protein, 18 aa, Artificial Sequence
  Albumin signal peptide
MKWVTFISLLFLFSSAYS

SEQ ID NO: 23 - protein, 18 aa, Artificial Sequence
  Chymotrypsinogen signal peptide
MAFLWLLSCWALLGTTFG

SEQ ID NO: 24 - protein, 20 aa, Artificial Sequence
  Interleukin-2 signal peptide
MYRMQLLSCIALILALVTNS

SEQ ID NO: 25 - protein, 15 aa, Artificial Sequence
  Trypsinogen-2 signal peptide
MNLLLILTFVAAAVA

SEQ ID NO: 26 - protein, 19 aa, Artificial Sequence
  F2A site
LLNFDLLKLAGDVESNPGP

SEQ ID NO: 27 - protein, 21 aa, Artificial Sequence
  Linker T2A
GSGEGRGSLLTCGDVEENPGP

SEQ ID NO: 28 - protein, 22 aa, Artificial Sequence
  Linker P2A
GSGATNFSLLKQAGDVEENPGP

SEQ ID NO: 29 - protein, 23 aa, Artificial Sequence
  Linker E2A
GSGQCTNYALLKLAGDVESNPGP

SEQ ID NO: 30 - protein, 25 aa, Artificial Sequence
  Linker F2A
GSGVKQTLNFDLLKLAGDVESNPGP

SEQ ID NO: 31 - protein, 4 aa, Artificial Sequence
  Furin linker
RKRR

SEQ ID NO: 32 - protein, 4 aa, Artificial Sequence
  Furin linker
RRRR

SEQ ID NO: 33 - protein, 4 aa, Artificial Sequence
  Furin linker
RRKR

SEQ ID NO: 34 - protein, 4 aa, Artificial Sequence
  Furin linker
RKKR

SEQ ID NO: 35 - protein, 4 aa, Artificial Sequence
  Furin linker
  MISC_FEATURE (2)..(2): X = any amino acid
  MISC_FEATURE (3)..(3): X = Lys or Arg
RXXR

SEQ ID NO: 36 - protein, 4 aa, Artificial Sequence
  Furin linker
  MISC_FEATURE (2)..(2): X = any amino acid
RXKR

SEQ ID NO: 37 - protein, 4 aa, Artificial Sequence
  Furin linker
  MISC_FEATURE (2)..(2): X = any amino acid
RXRR

SEQ ID NO: 38 - protein, 215 aa, Artificial Sequence
  Ranibizumab Fab Amino Acid Sequence - light chain
MDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 236 aa, Artificial Sequence
  Ranibizumab Fab Amino Acid Sequence - heavy chain
MEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLRKRR

SEQ ID NO: 40 - protein, 232 aa, Artificial Sequence
  Ranibizumab Fab Amino Acid Sequence - heavy chain
MEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 41 - protein, 736 aa, Artificial Sequence
  AAV1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 42 - protein, 735 aa, Artificial Sequence [sequence omitted]

SEQ ID NO: 43 - protein, 736 aa, Artificial Sequence
  AAV3-3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 44 - protein, 734 aa, Artificial Sequence
  AAV4-4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 45 - protein, 724 aa, Artificial Sequence
  AAV5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 46 - protein, 736 aa, Artificial Sequence
  AAV6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 47 - protein, 737 aa, Artificial Sequence
  AAV7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 48 - protein, 738 aa, Artificial Sequence
  AAV8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 49 - protein, 736 aa, Artificial Sequence
  hu31
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 50 - protein, 736 aa, Artificial Sequence
  hu32
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 51 - protein, 736 aa, Artificial Sequence
  AAV9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 52 - protein, 191 aa, Artificial Sequence
  Vascular endothelial growth factor (vegf) Caa44447.1
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 53 - protein, 306 aa, Artificial Sequence
  Palmitoyl-protein thioesterase 1 (ppt1) Aah08426.1
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 54 - protein, 563 aa, Artificial Sequence
  Tripeptidyl-peptidase 1 (tpp1) Np_000382.3
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 55 - protein, 20 aa, Artificial Sequence
  Mutant interleukin-2 signal peptide
MYRMQLLLLIALSLALVTNS